(19) United States
(12) Patent Application Publication
(10) Pub. No.: US 2009/0312306 A1
(43) Pub. Date: Dec. 17, 2009

(54) TETRAHYDRO-5H-PYRIDO[2,3-D]AZEPINES AS 5-HT2C LIGANDS

(76) Inventors: Guangri Sun, Toronto (CA); Methvin Isaac, Toronto (CA); Tao Xin, Toronto (CA); Higgins Guy, North York (CA)

Correspondence Address: OHLANDT, GREELEY, RUGGIERO & PERLE, LLP, ONE LANDMARK SQUARE, 10TH FLOOR, STAMFORD, CT, 06901, US

(21) Appl. No.: 12/309,446

(22) PCT Filed: Jul. 20, 2007
(86) PCT No.: PCT/CA2007/001286

§ 371 Date: May 4, 2009

Related U.S. Application Data
(60) Provisional application No. 60/807,839, filed on Jul. 20, 2006.

(51) Int. Cl. A61K 31/55; C07D 471/04; A61P 25/00

(52) U.S. Cl. 514/215; 540/580; 540/575

(57) ABSTRACT

A compound of Formula I:

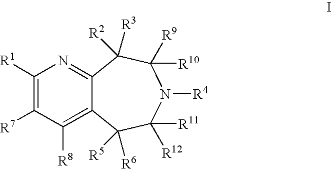

and a method for treating a 5-HT_{2C} receptor-mediated disorder in a mammal using the same.

### FIELD OF THE INVENTION

This invention relates to compounds which act at the 5-HT_{2c} receptor and to the use of such compounds in the treatment of diseases.

### BACKGROUND OF THE INVENTION

5-Hydroxytryptamine (5-HT or serotonin), a key neurotransmitter of the peripheral and central nervous system (PNS and CNS), has been implicated in a variety of sensory, motor and behavioral processes. The diverse effects of this neurotransmitter are related to the extensive projections of serotonergic neurons throughout the brain and the large number of distinct serotonin receptor subtypes. At least 14 distinct serotonin receptor subtypes are expressed in the mammalian CNS. The contribution of these receptors to the action of serotonin has been difficult to ascertain owing to the paucity of selective pharmacological agents.

The 5-HT₂ subfamily of serotonin receptors is composed of three subtypes; namely the 5-HT₂ₐ, 5-HT_{2b} and 5-HT_{2c} receptors. All the members of this subfamily couple to the activation of the inositol phosphate and diacyl glycerol pathway via the G-protein, G_{q/11}. Recently, other second messenger systems have been shown to be regulated by 5-HT₂ stimulation including mitogen activated protein kinase (MAP-kinase). The limited access to selective pharmacological tools amongst the 5-HT₂ subfamily of serotonin receptors has led to the use of gene targeting techniques to generate mouse lines that selectively lack functional receptor genes. This strategy has been applied to the study of 5-HT_{2c} receptor function. The 5-HT_{2c} receptor is expressed in many brain regions including the limbic system, extrapyramidal motor pathways, hypothalamus, thalamus and monoaminergic cell groups. 5-HT_{2c} receptors have been implicated in the regulation of food intake and anxiety. For example, the non-selective 5-HT_{2c} receptor agonist, m-chlorophenylpiperazine 1 (mCPP) produces hypophagic and anxiogenic effects that were attenuated by 5-HT_{2c} receptor antagonists. The propensity of a 5-HT_{2c} receptor agonist to regulate food intake suggests a critical role for this receptor subtype in controlling obesity (Vickers, S.; Clifton, P.; Dourish, C.; Tecott, L. Psychopharmacology (Berlin) 1999, 143, 309; Nilsson, B. J. Med. Chem. 2006, 49, 4023).

It has been widely recognized that obesity is a disease process influenced by environmental factors in which the traditional weight loss methods of dieting and exercise need to be supplemented by therapeutic products (S. Parker, ‘Obesity: Trends and Treatments”, Scrip Reports, PJB Publications Ltd, 1969).

Whether someone is classified as overweight or obese is generally determined on the basis of their body mass index (BMI), which is calculated by dividing body weight (kg) by height squared (m²). Thus, the units of BMI are kg/m² and it is possible to calculate the BMI range associated with minimum mortality in each decade of life. Overweight is defined as a BMI in the range 25-30 kg/m², and obesity as a BMI greater than 30 kg/m². There are problems with this definition in that it does not take into account the proportion of body mass that is muscle in relation to fat (adipose tissue). To account for this, obesity can also be defined on the basis of body fat content: greater than 25% and 30% in males and females, respectively. As the BMI increases there is an increased risk of death from a variety of causes that in independent of other risk factors. The most common diseases with obesity are cardiovascular disease (particularly hypertension), diabetes (obesity aggravates the development of diabetes), gall bladder disease (particularly cancer) and diseases of reproduction. Research has shown that even a modest reduction in body weight can correspond to a significant reduction in the risk of developing coronary heart disease.

In addition to its growing role in the regulation of food intake and hence obesity, the 5-HT_{2c} receptor has been implicated in the treatment of Schizophrenia. Schizophrenia affects approximately 5 million people. The most prevalent treatments for schizophrenia are currently the ‘atypical’ antipsychotics, which combine dopamine (D₂) and serotonin (5-HT_{2A}) receptor antagonism. Despite the reported improvements in efficacy and side-effect liability of atypical antipsychotics relative to typical antipsychotics, these compounds do not appear to adequately treat all the symptoms of schizophrenia and are accompanied by problematic side effects, such as weight gain (Allison, D. B., et. al., Am. J. Psychiatry, 156:1686-1696, 1999; Masand, P. S., Exp. Opin. Pharmacother. I:377-389, 2000; Whitaker, R., Spectrum Life Sciences. Decision Resources. 2:1-9, 2000).

Atypical antipsychotics also bind with high affinity to 5-HT_{2C} receptors and function as 5-HT_{2C} receptor antagonists or inverse agonists. Weight gain is a problematic side effect associated with atypical antipsychotics such as clozapine and olanzapine, and it has been suggested that 5-HT_{2C} antagonism is responsible for the increased weight gain. Conversely, stimulation of the 5-HT_{2C} receptor is known to result in decreased food intake and body weight (Walsh et. al., Psychopharmacology 124: 57-73, 1996; Cowen, P. J., et. al., Human Psychopharmacology 10: 385-391, 1995; Rosenzweig-Lipson, S., et. al., ASPET abstract, 2000).

Several lines of evidence support a role for 5-HT_{2C} receptor agonism or partial agonism as a treatment for schizophrenia. Studies suggest that 5-HT_{2C} antagonists increase synaptic levels of dopamine and may be effective in animal models of Parkinson's disease (Di Matteo, V., et. al., Neuropharmacology 37: 265-272, 1998; Fox, S. H., et. al., Experimental Neurology 151: 35-49, 1998). Since the positive symptoms of schizophrenia are associated with increased levels of dopamine, compounds with actions opposite to those of 5-HT_{2C} antagonists, such as 5-HT_{2C} agonists and partial agonists, should reduce levels of synaptic dopamine. Recent studies have demonstrated that 5-HT_{2C} agonists decrease levels of dopamine in the prefrontal cortex and nucleus accumbens (Millan, M. J., et. al., Neuropharmacology 37: 953-955, 1998; Di Matteo, V., et. al., Neuropharmacology 38: 1195-1205, 1999; Di Giovanni, G., et. al., Synapse 35: 53-61, 2000), brain regions that are thought to mediate critical antipsychotic effects of drugs like clozapine. However, 5-HT_{2C} agonists do not decrease dopamine levels in the striatum, the brain region most closely associated with extrapyramidal side effects. In addition, a recent study demonstrates that 5-HT_{2C} agonists decrease firing in the ventral tegmental area (VTA), but not in the substantia nigra. The differential effects of 5-HT_{2C} agonists in the mesolimbic pathway relative to the nigrostriatal pathway suggest that 5-HT_{2C} agonists have limbic selectivity, and will be less likely to produce extrapyramidal side effects associated with typical antipsychotics.

Additionally, 5-HT_{2C} receptors might also be involved in modulation of the rewarding properties of food, which is linked to increased mesolimbic dopamine levels in the nucleus accumbens of the brain in response to food ingestion. A number of studies have suggested that food and drug rewards may share some common neural substrates, specifically the nucleus accumbens (Saper, C. B.; Chou, T. C.; Elmquist, J. K. The need to feed: homeostatic and hedonic control of eating. Neuron 2002, 36, 199-211). Given that 5-HT_{2C} receptor agonists may decrease dopamine levels in the nucleus accumbens and that reward-related behaviors (e.g., cocaine or nicotine self-administration in rats) may be reduced by 5-HT_{2C} receptor activation, the possibility that 5-HT_{2C} receptor agonists may reduce the rewarding properties of food should also be considered (Higgins, G. A.; Fletcher, P. J. Serotonin and drug reward: focus on 5-HT_{2c} receptors. Eur. J. Pharmacol. 2003, 480, 151-162).

Another therapeutic area where the use of a 5-HT_{2C} receptor agonist is considered of value is in the treatment of epilepsy. Epilepsy, a brain disorder manifested by recurrent seizures, refers to a complicated constellation of more than 40 distinct disorders. The seizure, a sudden massive neuronal discharge, can be either partial or complete, depending on the area of brain involved or whether or not consciousness is impaired. Normally there is a balance between excitation and inhibition in the brain. When this balance is disrupted by increased excitation or decreased inhibition, a seizure may result. The neuronal discharges may stimulate muscles innervated by the nerves involved, resulting in involuntary muscle contractions, or convulsions (Lee, G. V.; Jones, E. J. Epilepsy. Neurobiology of Diseases 2000, 7, 549-551).

There are currently several drugs in clinical use to inhibit seizures, which fall into three different categories in terms of their target (Cosford, N. D. P.; McDonald, I. A.; Schweiger, E. J. Recent Progress in Antiepileptic Drug Research. Annu. Rep. Med. Chem. 1998, 33, 61-70). Most common are the drugs that affect the flow of sodium into the cell via voltage-gated sodium ion channels. A sodium ion channel is a structure in the cell membrane that is selectively permeable to sodium ions and is opened by changes in voltage across the cell membrane. Other drugs affect calcium ion channels. The third category of drugs affects some aspect of inhibitory synapses that are activated by the neurotransmitter γ-aminobutyric acid (GABA). Despite the availability of these drugs, a large proportion of patients continue to have seizures. Furthermore, among those in whom seizures are effectively inhibited, substantial numbers experienced persistent and undesirable effects from these drugs. In light of this, the current goal of researchers is to identify new classes of anti-seizure drugs that act on novel molecular targets and by novel mechanisms that may permit effective treatment of large numbers of individuals unsatisfactorily treated at present. The recently cloned 5-HT_{2C} receptor has revealed a novel molecular target that provides just this opportunity for the development of novel antiepileptic drugs.

There is growing evidence that serotonergic neurotransmission modulates a wide variety of experimentally-induced seizures and involved in the enhanced seizure susceptibility observed in some genetically prone rodents Przegalinski, E.; Baran, L.; Siwanowicz, J. role of 5-hydroxytryptamine receptor subtypes in the 1-[3-(trifluoromethyl)phenyl]piperazine-induced increase in threshold for maximal electroconvulsions in mice. Epilepsia, 1994, 35, 889-894; Wada, Y.; Shiraishi, J.; Nakamura, M.; Koshino, Y. Role of serotonin receptor subtypes in the development of amygdaloid kindling in rats. Brain Res., 1997, 747, 338-342). Studies have shown that mice bearing a targeted disruption of the 5-HT_{2C} receptor genes exhibit an epilepsy syndrome associated with sporadic spontaneous seizures that occasionally result in death. In all epileptic paradigms, mice lacking the 5-HT_{2C} receptors were significantly more seizure susceptible than wild-type controls. Results indicate that mutants have lower focal seizure thresholds, increased focal seizure excitability, and facilitated propagation within the forebrain seizure system. Mutants also exhibit lower generalized seizure threshold for the expression of both generalized clonic and generalized tonic seizures. Importantly, the 5-HT receptor antagonist, mesulergine (2 or 4 mg/kg), administered prior to electroshock testing, recapitulated the mutant phenotype in wild-type mice. Together, these data strongly implicate a role for serotonin and the 5-HT_{2C} receptors in the modulation of neuronal network excitability and seizure propagation throughout the CNS (Appelgate, C. D.; Tecoft, L. H. Global increases in seizure susceptibility in mice lacking 5-HT2C receptors; a behavioral analysis. Exp. Neurol. 1998, 154, 522-530; Heisler, L. K.; Chu, H. M.; Tecott, L. H. Epilepsy and obesity in serotonin 5-HT_{2C} receptor mutant mice. Ann. N.Y. Acad. Sci. 1998, 861, 74-78; Rueter, L. E.; Tecott, L. H.; Blier, P. In vivo electrophysiological examination of 5-HT responses in 5-HT_{2C} receptor mutant mice. Naunyn-Schmiedeberg's Arch. Pharmacol. 2000, 361, 484-491). Furthermore, agents that elevate extracellular serotonin (5-HT) levels, such as 5-hydroxytryptophan and 5-HT reuptake blockers, inhibit both limbic and generalized seizures. Conversely, depletion of brain 5-HT lowers the threshold to audiogenically, chemically and electrically evoked convulsion (Loscher, W.; Genetic animal models of epilepsy as a unique resource for the evaluation of anticonvulsant drugs. A review. Methods Find Exp. Clin. Pharmacol., 1984, 6, 531-547; Prendiville, S.; Gale, K. Anticonvulsant effect of fluoxetine on focally evoked limbic motor seizures in rats. Epilepsia, 1993, 34, 381-384; Yan, Q. S.; Jobe, P. C.; Cheong, J. H.; Ko, K. H.; Daily, J. W. Role of serotonin in the anticonvulsant effect of fluoxetine in genetically epilepsy-prone rats. Naunyn-Schmiedeberg's Arch. Pharmacol., 1994, 350, 149-152).

Reduction in seizure activity has been observed for the 5-HT_{2C} receptor agonists mCPP and TFMPP when microinjected bilaterally into the rat substantia nigra. This indicates that the 5-HT_{2C} receptors in the substantia nigra may contribute to seizure regulation (Gobert, A.; Rivet, J.; Lejeune, F.; et al. Serotonin (2C) receptors tonically suppress the activity of mesocortical dopaminergic and adrenergic, but not serotonergic pathways: a combined dialysis and electrophysiological analysis in the rat. Synapse 2000, 36, 205-221; Hutson, P. H.; Barton, C. L.; Jay, M.; et al. Activation of mesolimbic dopamine function by phencyclidine is enhanced by 5-HT_{2C/2B} receptor antagonists: neurochemical and behavioural studies. Neuropharmacology 2000, 39, 2318-2328). Among the clinically effective anticonvulsants such as carbamazepine, dose-related anticonvulsant effects correlate with increased extracellular serotonin further implicating the role of serotonin and hence the 5-HT_{2C} receptor agonist in epileptic seizures. Nevertheless, cross talk between the 5-HT_{2C} and γ-amino butyric acid (GABA) receptors in the mediation of the observed anticonvulsant activity should not be overlooked (Huidobro-Toro, J. P.; Valenzuela, C. F.; Harris, R. A. Modulation of GABAA receptor function by G protein-coupled 5-HT2C receptors. Neuropharmacology 1996, 35, 1355-1363).

Despite the fact that a large number of 5-HT receptors with different anatomical localization and function have been identified, there are only few studies investigating the role of 5-HT receptor subtypes in the modulation of seizure activity and the results are sometimes controversial depending on the experimental epilepsy model used (Jakus, R.; Graf, M.; Juhasz, G.; Geber, K.; Evay, G.; Halasz, P.; Bagdy, G. 5-HT_{2C} receptors inhibit and 5-HT_{1A} receptors activate the generation of spike-wave discharges in a genetic rat model of absence epilepsy. Exp. Neurol. 2003, 184, 964-972). In order to further delineate the role of the 5-HT_{2C} receptors in seizure generation, the effects of the 5-HT_{2C} preferring agonist, mCPP, were evaluated in a genetic absence epilepsy model. mCPP weakly elevated seizure threshold in mice (but not in rats) electroshock test, however appreciable protection against pentylenetetrazol-induced myoclonic and/or tonic seizures in mice and rats were observed. This protection against pentylenetetrazol-induced myoclonic and/or tonic seizures in mice and rats was inhibited by the 5-HT_{2C/2B} receptor antagonist, SB 206533. The fact that the 5-HT_{2B} agonist, BW-723C86, had no effect on animal seizure models supports the view that the 5-HT_{2C} receptor mediated the mCPP-induced anticonvulsant effects (Upton, N.; Middlemiss, D.; Blackburn, T.; et al. Studies on the role of 5-HT_{2C} and 5-HT_{2B} receptors in regulating generalized seizure threshold in rodents. Eur. J. Pharmacol. 1998, 359, 33-40). The selective 5-HT_{2C} receptor antagonist, SB 242084 do not induce pro-convulsant effects in rats, which are characteristic of mutant mice lacking the 5-HT_{2C} receptor. This failure to exhibit pro-convulsant properties in rats in contrast to the reported characteristics of mutant mice lacking 5-HT_{2C} receptors might be accounted for by species differences (Di Matteo, V.; Di Giovanni, G.; Eposito, E. SB-242084: A Selective 5-HT_{2C} Antagonist. CNS Drug Rev. 2000, 6, 195-205). Curiously, given the link between transferrin and the 5-HT_{2C} receptor, it would be of interest to study whether other transport proteins synthesized in the choroid plexus, in particular transthyretin (formerly called prealbumin), also are modulated by 5-HT_{2C} receptors. While speculative, this may be relevant for research on Alzheimer's disease (AD) because independent studies have indicated that both 5-HT_{2C} receptor agonism and transthyretin may reduce the amyloidogenic cleavage of the amyloid precursor protein (APP), a cleavage that produces neurotoxic β-amyloid protein, the principal proteinaceous component of brain amyloid plaques characteristic of AD (Arjona, A. A.; Pooler, A. M.; Lee, R. K.; Wurtman, R. J. Effect of a 5-HT_{2C} serotonin agonist, dexnorfenfluramine, on amyloid precursor protein metabolism in guinea pigs. Brain Res. 2002, 951, 135-140; Stein, T. D.; Anders, N. J.; DeCarli, C.; Chan, S. L.; Mattson, M. P.; Johnson, J. A. Neutralization of transthyretin reverses the neuroprotective effects of secreted amyloid precursor protein (APP) in APP_{Sw} mice resulting in tau phosphorylation and loss of hippocampal neurons: support for the amyloid hypothsis. J. Neurosci. 2004, 24, 7707-7717).

In recent years, several case reports of the efficacy of psilocybin in the treatment of obsessive-compulsive disorder (OCD) have been published (Delgado, P. L.; Moreno, F. A. J. Psychoactive Drugs 1998, 30, 359). As a result, an FDA-approved clinical trial for patients suffering from OCD is now underway (Sard, H.; Kumaran, G.; Morency, C.; Roth, B. L.; Toth, B.; He, P.; Shuster, L. Bioorg. Med. Chem. Lett. 2005, 15, 4555). The hallucinogenic activity of psilocybin and psilocin is believed to be largely due to activation of 5-HT_{2A} receptors, while the anti-OCD activity is associated with agonist activity at 5-HT_{2C}. Thus, it is believed that a selective 5-HT_{2C} agonist would have considerable potential for treatment of OCD (Roth, B. L.; Shapiro, D. Expert Opin. Ther. Targets 2001, 5, 685).

Selective serotonin reuptake inhibitors (SSRIs) increase extracellular levels of serotonin (5HT) and thereby nonselectively cause stimulation of all postsynaptic 5HT receptor subtypes. SSRIs have become standard therapy for neuropsychiatric disorders such as obsessive compulsive disorder (OCD), depression, and panic anxiety. There is accumulating evidence for the involvement of 5HT_{2C} receptor-mediated functions in the therapeutic efficacy of SSRIs (Pälvimäki, E.-P.; Roth, B. L.; Majasuo, H.; Laakso, A.; Kuoppamäki, M.; Syvälahti, E.; Hietala, J. Interactions of selective serotonin reuptake inhibitors with the serotonin 5HT_{2C} receptor. Psychopharmacology 1996, 126, 234-240; Jenck, F.; Moreau, J.-L.; Mutel, V.; Martin, J. R.; Haefely, W. E. Evidence for a role of 5HT_{1C} receptors in the antiserotonergic properties of some antidepressant drugs. Eur. J. Pharmacol. 1993, 231, 223-229). The increased 5HT synaptic content resulting from the reuptake inhibition also allows 5HT to act on the other 5HT receptor subtypes, possibly explaining some of the side effects associated with SSRI treatment. Selective 5HT_{2C} receptor agonists, therefore, may represent a direct means to produce the beneficial therapeutic effects of SSRIs without concomitant side effects.

Although these studies implicated the 5-HT_{2c} receptors in the modulation of feeding (obesity), schizophrenia, epilepsy, OCD and other related disorders, elucidation of the functional roles of these receptors has been hindered by a limited availability of selective agents. In addition, such paucity of selective agents can be attributed to the fact that the 5-HT_{2c} receptors share substantial sequence homology with the 5-HT₂ₐ and 5-HT_{2b} receptor.

### SUMMARY OF THE INVENTION

In one aspect, there is provided a compound of Formula I:

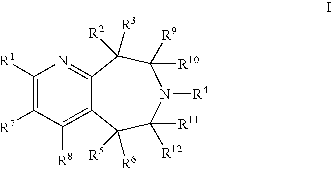

wherein:
R¹ to R³ and R⁵ to R¹² are independently selected from H, halo, hydroxy, cyano, nitro, alkyl, alkoxy, CH₂OH, haloalkyl, O-haloalkyl, hydroxyalkyl, cyanoalkyl, alkenyl, alkynyl, cycloalkyl, cycloalkenyl, heterocycloalkyl, heterocycloalkenyl, aryl, heteroaryl, alkylaryl, alkylheteroaryl, O-cycloalkyl, O-heterocycloalkyl, alkylene-O-alkyl, alkylene-O-cycloalkyl, alkylene-O-heterocycloalkyl, alkylene-O-alkylene-cycloalkyl, alkylene-O-alkylene-heterocycloalkyl, S-alkyl, S(O)-alkyl, S(O)₂-alkyl, S-cycloalkyl, S(O)-cycloalkyl, S(O)₂-cycloalkyl, S-heterocycloalkyl, S(O)-heterocycloalkyl, S(O)₂-heterocycloalkyl, O-aryl, O-heteroaryl, N(H)alkyl, N(alkyl)alkyl, N(H)-aryl, N(alkyl)-aryl, N(H)-heteroaryl, N(alkyl)-heteroaryl, alkylene-O-aryl, alkylene-O-heteroaryl, alkylene-O-alkylene-aryl, alkylene-O-alkylene-heteroaryl, S-aryl, S-heteroaryl, S(O)-aryl, S(O)-heteroaryl, S(O)₂-aryl, S(O)₂-heteroaryl, C(O)alkyl, OC(O)alkyl, C(O)Oalkyl, C(O)N(H)alkyl, C(O)N(alkyl)alkyl, S(O)₂N(H)alkyl or S(O)₂N(alkyl)alkyl; R² and R³, R⁵ and R⁶, R⁹ and R¹⁰, and/or R¹¹ and R¹², together with the carbon atom to which they are attached, form a cycloalkyl group; and
R⁴ is selected from H, alkyl, alkenyl, alkynyl, cycloalkyl, cycloalkenyl, alkylene-O-alkyl, alkylene-O-cycloalkyl, alkylene-O-alkylene-cycloalkyl;
and/or a pharmaceutically-acceptable salt, hydrate, solvate, isoform, tautomer, optical isomer, or combination thereof.

In another aspect, any cyclic group is substituted with one or more R¹³, R¹³ being selected from F, Cl, Br, I, CN, nitro, hydroxy, oxo, C₁₋₆-alkyl, OC₁₋₆-alkyl, C₁₋₆-alkylhalo or OC₁₋₆-alkylhalo.

In a further aspect, R⁴ is selected from H, alkyl, cycloalkyl, or cycloalkenyl.

In yet a further aspect, R⁴ is selected from H or alkyl.

In a further aspect, R⁹ to R¹² are independently selected from H, alkyl, alkenyl, alkynyl, cycloalkyl, cycloalkenyl, aryl, heteroaryl, alkylaryl, or alkylheteroaryl.

In yet a further aspect, R⁹ to R¹² are independently selected from H, alkyl or cycloalkyl.

In a further aspect, the compound is a pharmaceutically-acceptable salt, optical isomer, or combination thereof.

In a further aspect, the pharmaceutically-acceptable salt comprises an acid addition salt or a basic addition salt.

In a further aspect, the acid addition salt is formed from hydrochloric acid, hydrobromic acid, sulfuric acid, phosphoric acid, acid metal salt, monocarboxylic acids, dicarboxylic acids, or tricarboxylic acids.

In yet a further aspect, the compound of Formula I comprises a compound of Formula IA:

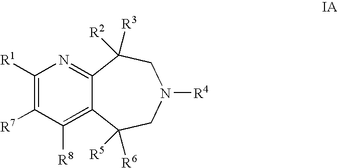

wherein:
R² and R⁵ are independently selected from H, alkyl, alkylene-O-alkyl, C(O)Oalkyl, C(O)N(H)alkyl, haloalkyl, halogen or CH₂OH; and
R³ and R⁶ are each H; or R² and R³ and/or R⁵ and R⁶, together with the carbon atom to which they are attached form a cycloalkyl group;
and/or a pharmaceutically-acceptable salt, hydrate, solvate, isoform, tautomer, optical isomer, or combination thereof.

In a further aspect, R² and R⁵ are independently selected from CH₂F, CHF₂, or CF₃.

In a further aspect, the compound of Formula I comprises a compound of Formula IB:

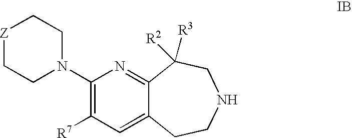

wherein:
Z is selected from CR¹⁴R¹⁵, O, NR⁶, C═O, S═O, SO₂ or S; and
R¹⁴ to R¹⁶ are independently selected from H, halo, hydroxy, cyano, nitro, alkyl, alkoxy, CH₂OH, haloalkyl, O-haloalkyl, hydroxyalkyl, cyanoalkyl, alkenyl, alkynyl, cycloalkyl, cycloalkenyl, heterocycloalkyl, heterocycloalkenyl, aryl, heteroaryl, alkylaryl, alkylheteroaryl, O-cycloalkyl, O-heterocycloalkyl, alkylene-O-alkyl, alkylene-O-cycloalkyl, alkylene-O-heterocycloalkyl, alkylene-O-alkylene-cycloalkyl, alkylene-O-alkylene-heterocycloalkyl, S-alkyl, S(O)-alkyl, S(O)₂-alkyl, S-cycloalkyl, S(O)-cycloalkyl, S(O)₂-cycloalkyl, S-heterocycloalkyl, S(O)-heterocycloalkyl, S(O)₂-heterocycloalkyl, O-aryl, O-heteroaryl, N(H)alkyl, N(alkyl)alkyl, N(H)-aryl, N(alkyl)-aryl, N(H)-heteroaryl, N(alkyl)-heteroaryl, alkylene-O-aryl, alkylene-O-heteroaryl, alkylene-O-alkylene-aryl, alkylene-O-alkylene-heteroaryl, S-aryl, S-heteroaryl, S(O)-aryl, S(O)-heteroaryl, S(O)₂-aryl, S(O)₂-heteroaryl, C(O)alkyl, OC(O)alkyl, C(O)Oalkyl, C(O)N(H)alkyl, C(O)N(alkyl)alkyl, S(O)₂N(H)alkyl or S(O)₂N(alkyl)alkyl;
and/or a pharmaceutically-acceptable salt, hydrate, solvate, isoform, tautomer, optical isomer, or combination thereof. In a further aspect, R⁷ is selected from H or halo; R² and R³ are independently selected from H or alkyl; R¹⁴ and R¹⁵ are independently selected from H, halo, or alkyl; and R¹⁶ is selected from H or alkyl. In a further aspect, the halo is bromo, chloro, or fluoro. In a further aspect, Z is CR¹⁴R¹⁵, wherein R¹⁴ is H or fluoro and R¹⁵ is fluoro. In a further aspect, R⁷ is H and Z is CR¹⁴R¹⁵, wherein R⁴ is H and R¹⁵ is fluoro.

In a further aspect, the compound of Formula I comprises a compound of Formula IC:

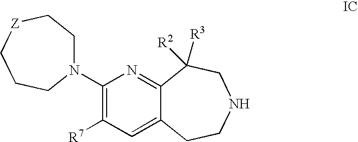

wherein:
Z is selected from CR¹⁴R¹⁵, O, NR¹⁶, C═O, S═O, SO₂ or S; and
R¹⁴ to R¹⁶ are independently selected from H, halo, hydroxy, cyano, nitro, alkyl, alkoxy, CH₂OH, haloalkyl, O-haloalkyl, hydroxyalkyl, cyanoalkyl, alkenyl, alkynyl, cycloalkyl, cycloalkenyl, heterocycloalkyl, heterocycloalkenyl, aryl, heteroaryl, alkylaryl, alkylheteroaryl, O-cycloalkyl, O-heterocycloalkyl, alkylene-O-alkyl, alkylene-O-cycloalkyl, alkylene-O-heterocycloalkyl, alkylene-O-alkylene-cycloalkyl, alkylene-O-alkylene-heterocycloalkyl, S-alkyl, S(O)-alkyl, S(O)₂-alkyl, S-cycloalkyl, S(O)-cycloalkyl, S(O)₂-cycloalkyl, S-heterocycloalkyl, S(O)-heterocycloalkyl, S(O)₂-heterocycloalkyl, O-aryl, O-heteroaryl, N(H)alkyl, N(alkyl)alkyl, N(H)-aryl, N(alkyl)-aryl, N(H)-heteroaryl, N(alkyl)-heteroaryl, alkylene-O-aryl, alkylene-O-heteroaryl, alkylene-O-alkylene-aryl, alkylene-O-alkylene-heteroaryl, S-aryl, S-heteroaryl, S(O)-aryl, S(O)-heteroaryl, S(O)₂-aryl, S(O)₂-heteroaryl, C(O)alkyl, OC(O)alkyl, C(O)Oalkyl, C(O)N(H)alkyl, C(O)N(alkyl)alkyl, S(O)₂N(H)alkyl or S(O)₂N(alkyl)alkyl;
and/or a pharmaceutically-acceptable salt, hydrate, solvate, isoform, tautomer, optical isomer, or combination thereof. In a further aspect, R⁷ is selected from H or halo; R² and R³ are independently selected from H or alkyl; R¹⁴ and R¹⁵ are independently selected from H, halo, or alkyl; and R¹⁶ is selected from H or alkyl. In a further aspect, the halo is bromo, chloro, or fluoro. In a further aspect, Z is O.

In a further aspect, the compound of Formula I comprises a compound of Formula II:

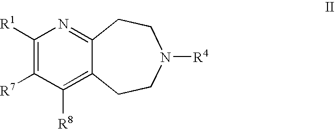

R¹, R⁴, R⁷ and R⁸ are as defined hereinabove.

In a further aspect, the compound of Formula I comprises a compound of Formula III:

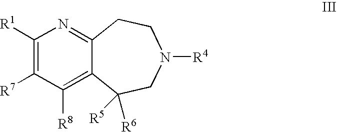

R¹, R⁴, R⁵, R⁶, R⁷ and R⁸ are as defined hereinabove.

In a further aspect, the compound of Formula I comprises a compound of Formula IV:

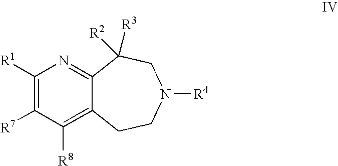

R¹, R², R³, R⁴, R⁷ and R⁸ are as defined hereinabove.

In a further aspect, R¹ is selected from H, alkyl or halo; R², R³ and R⁸ are independently selected from H or alkyl; R⁴ is selected from H or alkyl; and R⁷ is selected from H, alkyl, alkoxy, CH₂OH, alkenyl, cycloalkyl, heterocycloalkyl, aryl, or heteroaryl.

In a further aspect, there is provided a compound selected from:
(9R)-2-(4,4-difluoropiperidin-1-yl)-9-methyl-6,7,8,9-tetrahydro-5H-pyrido[2,3d]azepine;
(9R)-2-(4-fluoropiperidin-1-yl)-9-methyl-6,7,8,9-tetrahydro-5H-pyrido[2,3-d]azepine;
(9R)-9-methyl-2-(1,4-oxazepan-4-yl)-6,7,8,9-tetrahydro-5H-pyrido[2,3-d]azepine;
(9R)-9-methyl-2-morpholin-4-yl-6,7,8,9-tetrahydro-5H-pyrido[2,3-d]azepine;
(9R)-9-methyl-2-piperidin-1-yl-6,7,8,9-tetrahydro-5H-pyrido[2,3-d]azepine;
(9R)-N-ethyl-N,9-dimethyl-6,7,8,9-tetrahydro-5H-pyrido[2,3-d]azepin-2-amine;
(9S)-2-(4,4-difluoropiperidin-1-yl)-9-methyl-6,7,8,9-tetrahydro-5H-pyrido[2,3-d]azepine;
(9S)-2-(4-fluoropiperidin-1-yl)-9-methyl-6,7,8,9-tetrahydro-5H-pyrido[2,3-d]azepine;
(9S)-9-methyl-2-(1,4-oxazepan-4-yl)-6,7,8,9-tetrahydro-5H-pyrido[2,3-d]azepine
(9S)-9-methyl-2-morpholin-4-yl-6,7,8,9-tetrahydro-5H-pyrido[2,3-d]azepine;
(9S)-9-methyl-2-piperidin-1-yl-6,7,8,9-tetrahydro-5H-pyrido[2,3-d]azepine;
(9S)-N-ethyl-N,9-dimethyl-6,7,8,9-tetrahydro-5H-pyrido[2,3-d]azepin-2-amine;
2-(1,4-diazepan-1-yl)-6,7,8,9-tetrahydro-5H-pyrido[2,3-d]azepine;
2-(1,4-oxazepan-4-yl)-6,7,8,9-tetrahydro-5H-pyrido[2,3-d]azepine;
2-(1-oxidothiomorpholin-4-yl)-6,7,8,9-tetrahydro-5H-pyrido[2,3-d]azepine;
2-(3-thienyl)-6,7,8,9-tetrahydro-5H-pyrido[2,3-d]azepine;
2-(4,4-difluoroazepan-1-yl)-6,7,8,9-tetrahydro-5H-pyrido[2,3-d]azepine;
2-(4,4-difluoroazepan-1-yl)-9-methyl-6,7,8,9-tetrahydro-5H-pyrido[2,3-d]azepine;
2-(4,4-difluoropiperidin-1-yl)-6,7,8,9-tetrahydro-5H-pyrido[2,3-d]azepine;
2-(4,4-difluoropiperidin-1-yl)-9-methyl-6,7,8,9-tetrahydro-5H-pyrido[2,3-d]azepine;
2-(4-fluoropiperidin-1-yl)-6,7,8,9-tetrahydro-5H-pyrido[2,3-d]azepine;
2-(4-fluoropiperidin-1-yl)-9-methyl-6,7,8,9-tetrahydro-5H-pyrido[2,3-d]azepine;
2-(4-methylpiperazin-1-yl)-6,7,8,9-tetrahydro-5H-pyrido[2,3-d]azepine;
2-(8-azabicyclo[3.2.1]oct-8-yl)-6,7,8,9-tetrahydro-5H-pyrido[2,3-d]azepine;
2-(trifluoromethyl)-6,7,8,9-tetrahydro-5H-pyrido[2,3-d]azepine;
2,9-dimethyl-6,7,8,9-tetrahydro-5H-pyrido[2,3-d]azepine;
2-[methyl(6,7,8,9-tetrahydro-5H-pyrido[2,3-d]azepin-2-yl)amino]ethanol;
2-azepan-1-yl-6,7,8,9-tetrahydro-5H-pyrido[2,3-d]azepine;
2-azepan-1-yl-9-isopropyl-6,7,8,9-tetrahydro-5H-pyrido[2,3-d]azepine;
2-azepan-1-yl-9-methyl-6,7,8,9-tetrahydro-5H-pyrido[2,3-d]azepine;
2-cyclopropyl-6,7,8,9-tetrahydro-5H-pyrido[2,3-d]azepine;
2-cyclopropyl-9-methyl-6,7,8,9-tetrahydro-5H-pyrido[2,3-d]azepine;
2-isopropenyl-6,7,8,9-tetrahydro-5H-pyrido[2,3-d]azepine;
2-isopropenyl-9-methyl-6,7,8,9-tetrahydro-5H-pyrido[2,3-d]azepine;
2-isopropyl-6,7,8,9-tetrahydro-5H-pyrido[2,3-d]azepine;
2-isopropyl-9-methyl-6,7,8,9-tetrahydro-5H-pyrido[2,3-d]azepine;
2-methoxy-6,7,8,9-tetrahydro-5H-pyrido[2,3-d]azepine;
2-methoxy-9-methyl-6,7,8,9-tetrahydro-5H-pyrido[2,3-d]azepine;
2-methyl-6,7,8,9-tetrahydro-5H-pyrido[2,3-d]azepine;
2-morpholin-4-yl-6,7,8,9-tetrahydro-5H-pyrido[2,3-d]azepine;
2-piperazin-1-yl-6,7,8,9-tetrahydro-5H-pyrido[2,3-d]azepine;
2-piperidin-1-yl-6,7,8,9-tetrahydro-5H-pyrido[2,3-d]azepine;
2-pyrrolidin-1-yl-6,7,8,9-tetrahydro-5H-pyrido[2,3-d]azepine;
2-tert-butyl-6,7,8,9-tetrahydro-5H-pyrido[2,3-d]azepine;
2-thiomorpholin-4-yl-6,7,8,9-tetrahydro-5H-pyrido[2,3-d]azepine;
2-vinyl-6,7,8,9-tetrahydro-5H-pyrido[2,3-d]azepine;
3-bromo-2-methoxy-6,7,8,9-tetrahydro-5H-pyrido[2,3-d]azepine;
3-bromo-2-morpholin-4-yl-6,7,8,9-tetrahydro-5H-pyrido[2,3-d]azepine;
3-bromo-2-piperidin-1-yl-6,7,8,9-tetrahydro-5H-pyrido[2,3-d]azepine;
3-bromo-N-ethyl-N-methyl-6,7,8,9-tetrahydro-5H-pyrido[2,3-d]azepin-2-amine;
3-chloro-2-(1,4-oxazepan-4-yl)-6,7,8,9-tetrahydro-5H-pyrido[2,3-d]azepine;
3-chloro-2-(4,4-difluoropiperidin-1-yl)-6,7,8,9-tetrahydro-5H-pyrido[2,3-d]azepine;
3-chloro-2-(4-fluoropiperidin-1-yl)-6,7,8,9-tetrahydro-5H-pyrido[2,3-d]azepine;
3-chloro-2-morpholin-4-yl-6,7,8,9-tetrahydro-5H-pyrido[2,3-d]azepine;
3-chloro-2-piperidin-1-yl-6,7,8,9-tetrahydro-5H-pyrido[2,3-d]azepine;
6,7,8,9-tetrahydro-5H-pyrido[2,3-d]azepine-2-carbaldehyde;
6,7,8,9-tetrahydro-5H-pyrido[2,3-d]azepine-2-carbonitrile;
9-ethyl-2-morpholin-4-yl-6,7,8,9-tetrahydro-5H-pyrido[2,3-d]azepine;
9-ethyl-2-piperidin-1-yl-6,7,8,9-tetrahydro-5H-pyrido[2,3-d]azepine;
9-isopropyl-2-morpholin-4-yl-6,7,8,9-tetrahydro-5H-pyrido[2,3-d]azepine;
9-isopropyl-2-piperidin-1-yl-6,7,8,9-tetrahydro-5H-pyrido[2,3-d]azepine;
9-methyl-2-(1,4-oxazepan-4-yl)-6,7,8,9-tetrahydro-5H-pyrido[2,3-d]azepine;
9-methyl-2-morpholin-4-yl-6,7,8,9-tetrahydro-5H-pyrido[2,3-d]azepine;
9-methyl-2-piperidin-1-yl-6,7,8,9-tetrahydro-5H-pyrido[2,3-d]azepine;
ethyl 4-(6,7,8,9-tetrahydro-5H-pyrido[2,3-d]azepin-2-yl)piperazine-1-carboxylate;
N,9-diethyl-N-methyl-6,7,8,9-tetrahydro-5H-pyrido[2,3-d]azepin-2-amine;
N,N,9-trimethyl-6,7,8,9-tetrahydro-5H-pyrido[2,3-d]azepin-2-amine;
N,N-diallyl-6,7,8,9-tetrahydro-5H-pyrido[2,3-d]azepin-2-amine;
N,N-diethyl-6,7,8,9-tetrahydro-5H-pyrido[2,3-d]azepin-2-amine;
N,N-dimethyl-6,7,8,9-tetrahydro-5H-pyrido[2,3-d]azepin-2-amine;
N,N-dimethyl-6,7,8,9-tetrahydro-5H-pyrido[2,3-d]azepine-2-carboxamide;
N-benzyl-N-methyl-6,7,8,9-tetrahydro-5H-pyrido[2,3-d]azepin-2-amine;
N-ethyl-N,9-dimethyl-6,7,8,9-tetrahydro-5H-pyrido[2,3-d]azepin-2-amine;
N-ethyl-N-methyl-6,7,8,9-tetrahydro-5H-pyrido[2,3-d]azepin-2-amine;
N-isopropyl-N-methyl-6,7,8,9-tetrahydro-5H-pyrido[2,3-d]azepin-2-amine;
N-methyl-6,7,8,9-tetrahydro-5H-pyrido[2,3-d]azepin-2-amine;
N-methyl-N-(6,7,8,9-tetrahydro-5H-pyrido[2,3-d]azepin-2-yl)acetamide;
2-Phenyl-6,7,8,9-tetrahydro-5H-pyrido[2,3-d]azepine;
6,7,8,9-Tetrahydro-5H-pyrido[2,3-d]azepine-2-carbonitrile;
2-Chloro-6,7,8,9-tetrahydro-5H-pyrido[2,3-d]azepine;
[2-(6-Methoxy-3-methyl-pyridin-2-yl)-ethyl]-methyl-amine; and/or
7,8,9-Tetrahydro-5H-pyrido[2,3-d]azepin-2-ol;
   and/or a pharmaceutically-acceptable salt, hydrate, solvate, isoform, tautomer, optical isomer, or combination thereof.

In a further aspect, there is provided a compound selected from:
(9R)-2-(4,4-difluoropiperidin-1-yl)-9-methyl-6,7,8,9-tetrahydro-5H-pyrido[2,3d]azepine;
(9R)-2-(4-fluoropiperidin-1-yl)-9-methyl-6,7,8,9-tetrahydro-5H-pyrido[2,3-d]azepine;
(9R)-9-methyl-2-(1,4-oxazepan-4-yl)-6,7,8,9-tetrahydro-5H-pyrido[2,3-d]azepine;
(9R)-9-methyl-2-morpholin-4-yl-6,7,8,9-tetrahydro-5H-pyrido[2,3-d]azepine;
(9R)-9-methyl-2-piperidin-1-yl-6,7,8,9-tetrahydro-5H-pyrido[2,3-d]azepine;
(9R)-N-ethyl-N,9-dimethyl-6,7,8,9-tetrahydro-5H-pyrido[2,3-d]azepin-2-amine;
(9S)-2-(4,4-difluoropiperidin-1-yl)-9-methyl-6,7,8,9-tetrahydro-5H-pyrido[2,3-d]azepine;
(9S)-2-(4-fluoropiperidin-1-yl)-9-methyl-6,7,8,9-tetrahydro-5H-pyrido[2,3-d]azepine;
(9S)-9-methyl-2-(1,4-oxazepan-4-yl)-6,7,8,9-tetrahydro-5H-pyrido[2,3-d]azepine
(9S)-9-methyl-2-morpholin-4-yl-6,7,8,9-tetrahydro-5H-pyrido[2,3-d]azepine;
(9S)-9-methyl-2-piperidin-1-yl-6,7,8,9-tetrahydro-5H-pyrido[2,3-d]azepine;
(9S)-N-ethyl-N,9-dimethyl-6,7,8,9-tetrahydro-5H-pyrido[2,3-d]azepin-2-amine;
2-(1,4-diazepan-1-yl)-6,7,8,9-tetrahydro-5H-pyrido[2,3-d]azepine;
2-(1,4-oxazepan-4-yl)-6,7,8,9-tetrahydro-5H-pyrido[2,3-d]azepine;
2-(4,4-difluoroazepan-1-yl)-6,7,8,9-tetrahydro-5H-pyrido[2,3-d]azepine;
2-(4,4-difluoropiperidin-1-yl)-6,7,8,9-tetrahydro-5H-pyrido[2,3-d]azepine;
2-(4-fluoropiperidin-1-yl)-6,7,8,9-tetrahydro-5H-pyrido[2,3-d]azepine;
2-azepan-1-yl-6,7,8,9-tetrahydro-5H-pyrido[2,3-d]azepine;
2-azepan-1-yl-9-methyl-6,7,8,9-tetrahydro-5H-pyrido[2,3-d]azepine;
2-cyclopropyl-9-methyl-6,7,8,9-tetrahydro-5H-pyrido[2,3-d]azepine;
2-isopropenyl-6,7,8,9-tetrahydro-5H-pyrido[2,3-d]azepine;
2-isopropenyl-9-methyl-6,7,8,9-tetrahydro-5H-pyrido[2,3-d]azepine;
2-isopropyl-6,7,8,9-tetrahydro-5H-pyrido[2,3-d]azepine;
2-isopropyl-9-methyl-6,7,8,9-tetrahydro-5H-pyrido[2,3-d]azepine;
2-morpholin-4-yl-6,7,8,9-tetrahydro-5H-pyrido[2,3-d]azepine;
2-piperidin-1-yl-6,7,8,9-tetrahydro-5H-pyrido[2,3-d]azepine;
2-tert-butyl-6,7,8,9-tetrahydro-5H-pyrido[2,3-d]azepine;
2-thiomorpholin-4-yl-6,7,8,9-tetrahydro-5H-pyrido[2,3-d]azepine;
3-bromo-2-methoxy-6,7,8,9-tetrahydro-5H-pyrido[2,3-d]azepine;
3-chloro-2-(1,4-oxazepan-4-yl)-6,7,8,9-tetrahydro-5H-pyrido[2,3-d]azepine;
3-chloro-2-(4,4-difluoropiperidin-1-yl)-6,7,8,9-tetrahydro-5H-pyrido[2,3-d]azepine;
3-chloro-2-(4-fluoropiperidin-1-yl)-6,7,8,9-tetrahydro-5H-pyrido[2,3-d]azepine;
3-chloro-2-morpholin-4-yl-6,7,8,9-tetrahydro-5H-pyrido[2,3-d]azepine;
3-chloro-2-piperidin-1-yl-6,7,8,9-tetrahydro-5H-pyrido[2,3-d]azepine;
9-isopropyl-2-piperidin-1-yl-6,7,8,9-tetrahydro-5H-pyrido[2,3-d]azepine;
9-methyl-2-morpholin-4-yl-6,7,8,9-tetrahydro-5H-pyrido[2,3-d]azepine;
9-methyl-2-piperidin-1-yl-6,7,8,9-tetrahydro-5H-pyrido[2,3-d]azepine;
N,N-diethyl-6,7,8,9-tetrahydro-5H-pyrido[2,3-d]azepin-2-amine;
N,N-dimethyl-6,7,8,9-tetrahydro-5H-pyrido[2,3-d]azepin-2-amine;
N-ethyl-N,9-dimethyl-6,7,8,9-tetrahydro-5H-pyrido[2,3-d]azepin-2-amine; and/or
N-ethyl-N-methyl-6,7,8,9-tetrahydro-5H-pyrido[2,3-d]azepin-2-amine;
   and/or a pharmaceutically-acceptable salt, hydrate, solvate, isoform, tautomer, optical isomer, or combination thereof.

In yet another aspect, there is provided a compound according to any one of the compounds noted above, wherein the compound has an EC₅₀ for a human 5-HT_{2C} receptor selected from less than 1000 nM, less than 500 nM, less than 300 nM, or less than 100 nM.

In another aspect, there is provided a Boc-protected precusor of the compound according to any one of the compounds noted above, or mixtures thereof.

In another aspect, there is provided a pharmaceutical composition comprising at least one of the compounds noted above and at least one pharmaceutically acceptable carrier and/or excipient.

In a further aspect, there is provided a method for making the compound of Formula I, wherein R¹¹ and R¹² are H, the method comprising:

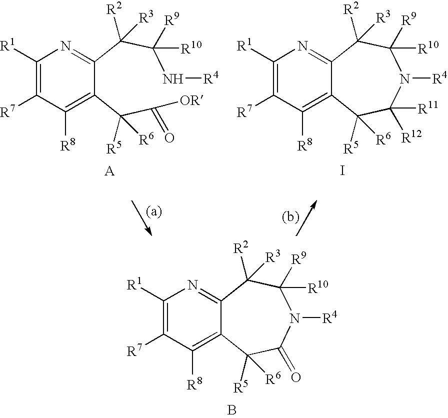

reacting a compound of Formula A under conditions (a), wherein said (a) comprises heat and base assisted cyclization of the compound of Formula A to provide an amide of Formula B; and
reducing the carbonyl of the amide of Formula B,
whereby R′ is alkyl or cycloalkyl.

In a further aspect, there is provided a method for making the compound of Formula I, wherein R⁹ and R¹⁰ are H, the method comprising:

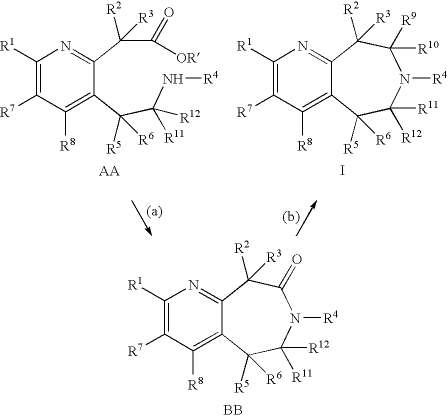

reacting a compound of Formula AA under conditions (a), wherein said (a) comprises heat and base assisted cyclization of the compound of Formula AA to provide an amide of Formula BB; and
reducing the carbonyl of the amide of Formula BB,
whereby R′ is alkyl or cycloalkyl.

In a further aspect, there is provided a method for making the compound of Formula I, wherein R¹¹ and R¹² are H, the method comprising:
reducing a carbonyl of an amide:

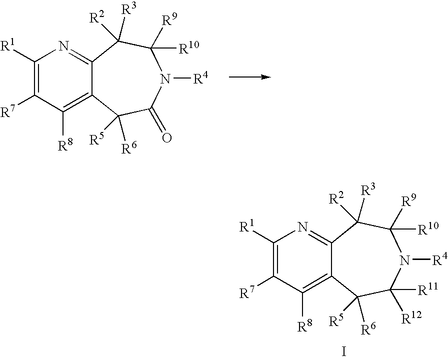

In a further aspect, there is provided a method for making the compound of Formula I, wherein R⁹ and R¹⁰ are H, the method comprising:
reducing a carbonyl of an amide:

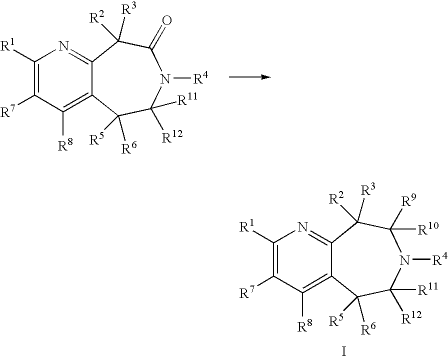

In a further aspect, there is provided a method for making the compound of Formula I, wherein R⁹, R¹⁰, R¹¹ and R¹² are H, the method comprising:
reducing carbonyl groups of an amide:

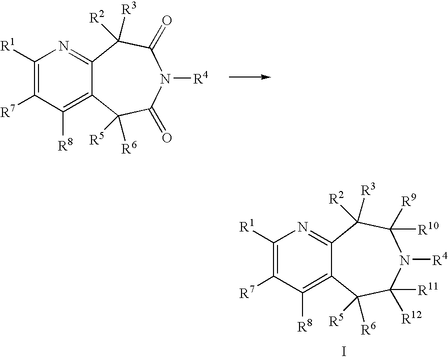

In a yet further aspect, there is provided a method for making the compound of Formula I, wherein R¹¹ and R¹² are H, the method comprising:

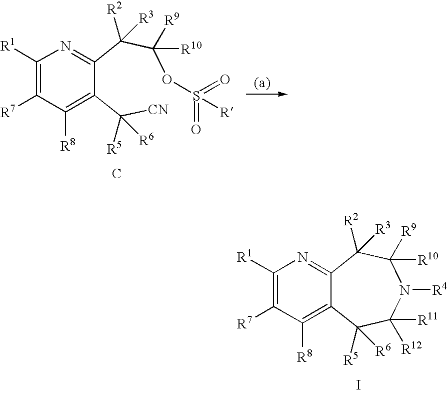

reacting a compound of Formula C under conditions (a), wherein said (a) comprises selective cyano reduction followed by cyclization of the compound of Formula C to provide Formula I, whereby R′ is alkyl or cycloalkyl.

In a yet further aspect, there is provided a method for making the compound of Formula I, wherein R⁹ and R¹⁰ are H, the method comprising:

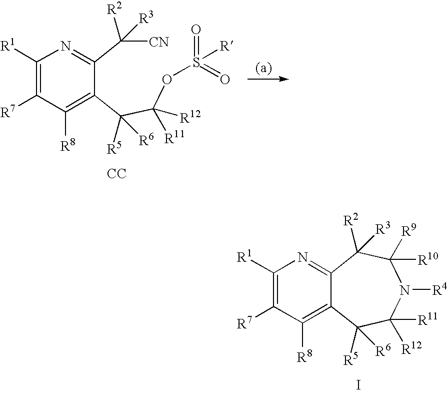

reacting a compound of Formula CC under conditions (a), wherein said (a) comprises selective cyano reduction followed by cyclization of the compound of Formula CC to provide Formula I, whereby R′ is alkyl or cycloalkyl.

In a yet further aspect, there is provided a method for making the compound of Formula I, wherein the method comprises:

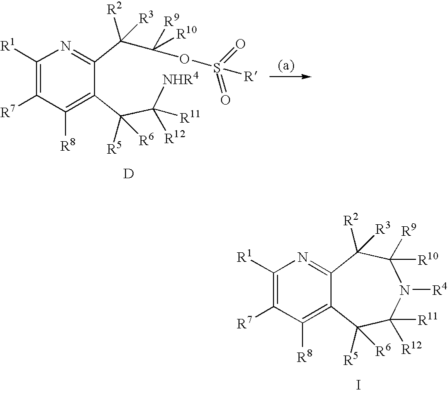

reacting a compound of Formula D under conditions (a), wherein said (a) comprises cyclization of the compound of Formula D to provide Formula I, whereby R′ is alkyl or cycloalkyl.

In a yet further aspect, there is provided a method for making the compound of Formula I, wherein the method comprises:

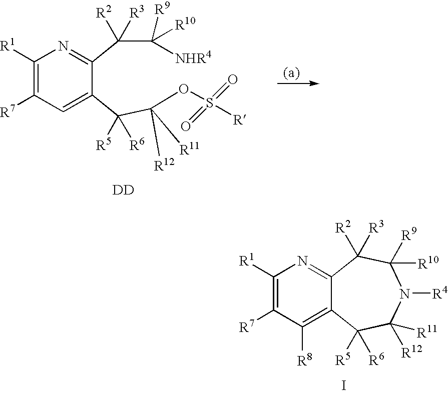

reacting a compound of Formula DD under conditions (a), wherein said (a) comprises cyclization of the compound of Formula DD to provide Formula I, whereby R′ is alkyl or cycloalkyl.

In another aspect, there is provided a method for treating a 5-HT_{2C} receptor-mediated disorder in a mammal, comprising administering to the mammal a therapeutically effective amount of a compound or composition noted above. In a further aspect, the mammal is a human. In another aspect, the disorder is selected from obesity, schizophrenia, epilepsy, depression, panic anxiety, alcoholism or obsessive compulsive disorder, a depressive disorder, an anxiety disorder, including panic attack, agoraphobia, and specific or social phobia, bipolar disorder, post-traumatic stress, an eating disorder, obesity, a gastrointestinal disorder, alcoholism, drug addiction, schizophrenia, a psychotic disorder, a sleep disorder, including sleep apnea, migraine, sexual dysfunction, a central nervous system disorder, including trauma, stroke and spinal cord injury, a cardio-vascular disorder, diabetes insipidus, obsessive compulsive disorder, premenstrual tension, chronic fatigue syndrome, age-related memory disorder, personality disorder and raised intracranial pressure. In a further aspect, the disorder is selected from obesity, schizophrenia, epilepsy, a depressive disorder, panic attack, alcoholism, drug addiction or obsessive compulsive disorder. In still a further aspect, the compound is administered orally and/or parenterally. In yet another aspect, the compound is administered intravenously and/or intraperitoneally.

In yet a further aspect, there is provided the use of the compound or composition noted above for the manufacture of a medicament for treatment of a 5-HT_{2C} receptor-mediated disease in a mammal. In yet a further aspect, there is provided the use of the compound or composition noted above to treat a 5-HT_{2C} receptor-mediated disease in a mammal. In a further aspect, the mammal is a human. In another aspect, the disorder is selected from obesity, schizophrenia, epilepsy, depression, panic anxiety, alcoholism or obsessive compulsive disorder, a depressive disorder, an anxiety disorder, including panic attack, agoraphobia, and specific or social phobia, bipolar disorder, post-traumatic stress, an eating disorder, obesity, a gastrointestinal disorder, alcoholism, drug addiction, schizophrenia, a psychotic disorder, a sleep disorder, including sleep apnea, migraine, sexual dysfunction, a central nervous system disorder, including trauma, stroke and spinal cord injury, a cardio-vascular disorder, diabetes insipidus, obsessive compulsive disorder, premenstrual tension, chronic fatigue syndrome, age-related memory disorder, personality disorder and raised intracranial pressure. In a further aspect, the disorder is selected from obesity, schizophrenia, epilepsy, a depressive disorder, panic attack, alcoholism, drug addiction or obsessive compulsive disorder. In still a further aspect, the compound is administrable orally and/or parenterally. In yet another aspect, the compound is administrable intravenously and/or intraperitoneally.

In another aspect, there is provided a method for decreasing food intake in a mammal comprising administering to the mammal a therapeutically effective amount of a compound or composition as noted above.

In another aspect, there is provided a method of controlling weight gain in a mammal comprising administering to the mammal a therapeutically effective amount of a compound or composition as noted above.

The novel features of the present invention will become apparent to those of skill in the art upon examination of the following detailed description of the invention. It should be understood, however, that the detailed description of the invention and the specific examples presented, while indicating certain embodiments of the present invention, are provided for illustration purposes only because various changes and modifications within the spirit and scope of the invention will become apparent to those of skill in the art from the detailed description of the invention and claims that follow.

### BRIEF DESCRIPTION OF THE DRAWINGS

Certain embodiments of the invention are described, reference being made to the accompanying drawings, wherein:

FIG. 1 shows graphically the effect of two exemplary compounds of the invention at various doses (mg/ml, X-axis), administered intraperitoneally (open bars) or orally (solid bars) on mouse locomotion expressed as % change control (Y axis).

FIG. 2 shows graphically the effect of two exemplary compounds of the invention at various doses (mg/ml) or vehicle administered intraperitoneally (X-axis) on rat food consumption (Y-axis). Hatched bars show pretreatment with 5-HT_{2c} antagonist SB242084 before compound administration.

### DEFINITIONS

Unless specified otherwise within this specification, the nomenclature used in this specification generally follows the examples and rules stated in Nomenclature of Organic Chemistry, Sections A, B, C, D, E, F, and H, Pergamon Press, Oxford, 1979, which is incorporated by references herein for its exemplary chemical structure names and rules on naming chemical structures. Optionally, a name of a compound may be generated using a chemical naming program: ACD/ChemSketch, Version 5.09/September 2001, Advanced Chemistry Development, Inc., Toronto, Canada.

The compounds of the present invention may have asymmetric centers, chiral axes, and chiral planes (as described in: E. L. Eliel and S. H. Wilen, Stereo-chemistry of Carbon Compounds, John Wiley & Sons, New York, 1994, pages 1119-1190), and occur as racemates, racemic mixtures, and as individual diastereomers, with all possible isomers and mixtures thereof, including optical isomers, being included in the present invention.

Generally, reference to a certain element such as hydrogen or H is meant to, if appropriate, include all isotopes of that element.

The following terms are meant to encompass unsubstituted and/or substituted.

The term “alkyl” as used herein means a straight- or branched-chain hydrocarbon radical; in one aspect, having from one to eight carbon atoms, and includes, for example, and without being limited thereto, methyl, ethyl, propyl, isopropyl, t-butyl and the like. As noted above, “alkyl” encompasses substituted alkyl. Substituted alkyl includes, for example, and without being limited thereto, haloalkyl, hydroxyalkyl, cyanoalkyl, and the like. This is applied to any of the groups mentioned herein. Groups such as “alkenyl”, “alkynyl”, “aryl”, etc. encompass substituted “alkenyl”, “alkynyl”, “aryl”, etc.

The term “alkenyl” as used herein means a straight- or branched-chain alkenyl radical; in one aspect, having from two to eight carbon atoms, and includes, for example, and without being limited thereto, ethenyl, 1-propenyl, 1-butenyl and the like. The term “alkenyl” encompass radicals having “cis” and “trans” orientations, or alternatively, “E” and “Z” orientations.

The term “alkynyl” as used herein means a straight- or branched-chain alkynyl radical; in one aspect, having from two to eight carbon atoms, and includes, for example, and without being limited thereto, 1-propynyl (propargyl), 1-butynyl and the like.

The term “cycloalkyl” as used herein means a carbocyclic system (which may be unsaturated) containing one or more rings wherein such rings may be attached together in a pendent manner or may be fused. In one aspect, the ring(s) may have from three to seven carbon atoms, and includes, for example, and without being limited thereto, cyclopropyl, cyclohexyl, cyclohexenyl and the like.

The term “heterocycloalkyl” as used herein means a heterocyclic system (which may be unsaturated) having at least one heteroatom selected from N, S and/or O and containing one or more rings wherein such rings may be attached together in a pendent manner or may be fused. In one aspect, the ring(s) may have a three- to seven-membered cyclic group and includes, for example, and without being limited thereto, piperidinyl, piperazinyl, pyrrolidinyl, tetrahydrofuranyl and the like.

The term “alkoxy” as used herein means a straight- or branched-chain alkoxy radical; in one aspect, having from one to eight carbon atoms and includes, for example, and without being limited thereto, methoxy, ethoxy, propyloxy, isopropyloxy, t-butoxy and the like.

The term “halo” as used herein means halogen and includes, for example, and without being limited thereto, fluoro, chloro, bromo, iodo and the like, in both radioactive and non-radioactive forms.

The term “alkylene” as used herein means a difunctional branched or unbranched saturated hydrocarbon radical; in one aspect, having one to eight carbon atoms, and includes, for example, and without being limited thereto, methylene, ethylene, n-propylene, n-butylene and the like.

The term “alkenylene” as used herein means a difunctional branched or unbranched hydrocarbon radical; in one aspect, having two to eight carbon atoms, and having at least one double bond, and includes, for example, and without being limited thereto, ethenylene, n-propenylene, n-butenylene and the like.

The term “alkynylene” as used herein means a difunctional branched or unbranched hydrocarbon radical; in one aspect, having two to eight carbon atoms, and having at least one triple bond, and includes, for example, and without being limited thereto, ethynylene, n-propynylene, n-butynylene and the like.

The term “aryl”, alone or in combination, as used herein means a carbocyclic aromatic system containing one or more rings wherein such rings may be attached together in a pendent manner or may be fused. In particular embodiments, aryl is one, two or three rings. In one aspect, the aryl has five to twelve ring atoms. The term “aryl” encompasses aromatic radicals such as phenyl, naphthyl, tetrahydronaphthyl, indanyl, biphenyl, phenanthryl, anthryl or acenaphthyl. The “aryl” group may have 1 to 4 substituents such as lower alkyl, hydroxyl, halo, haloalkyl, nitro, cyano, alkoxy, lower alkylamino and the like.

The term “heteroaryl”, alone or in combination, as used herein means an aromatic system having at least one heteroatom selected from N, S and/or O and containing one or more rings wherein such rings may be attached together in a pendent manner or may be fused. In particular embodiments, heteroaryl is one, two or three rings. In one aspect, the heteroaryl has five to twelve ring atoms. The term “heteroaryl” encompasses heteroaromatic radicals such as pyridyl, indolyl, furyl, benzofuryl, thienyl, benzothienyl, quinolyl, oxazolyl and the like. The “heteroaryl” group may have 1 to 4 substituents such as lower alkyl, hydroxyl, halo, haloalkyl, nitro, cyano, alkoxy, lower alkylamino and the like.

It is understood that substituents and substitution patterns on the compounds of the invention may be selected by one of ordinary skill in the art to provide compounds that are chemically stable and that can be readily synthesized by techniques known in the art, as well as those methods set forth below. If a substituent is itself substituted with more than one group, it is understood that these multiple groups may be on the same carbon or on different carbons, as long as a stable structure results.

The term “pharmaceutically acceptable salt” means either an acid addition salt or a basic addition salt which is compatible with the treatment of patients.

A “pharmaceutically acceptable acid addition salt” is any non-toxic organic or inorganic acid addition salt of the base compounds represented by Formula I or any of its intermediates. Illustrative inorganic acids which form suitable salts include, but are not limited thereto, hydrochloric, hydrobromic, sulfuric and phosphoric acid and acid metal salts such as sodium monohydrogen orthophosphate and potassium hydrogen sulfate. Illustrative organic acids which form suitable salts include the mono-, di- and tricarboxylic acids. Illustrative of such acids are, for example, acetic, glycolic, lactic, pyruvic, malonic, succinic, glutaric, fumaric, malic, tartaric, citric, ascorbic, maleic, hydroxymaleic, benzoic, hydroxybenzoic, phenylacetic, cinnamic, salicylic, 2-phenoxybenzoic, p-toluenesulfonic acid and other sulfonic acids such as methanesulfonic acid and 2-hydroxyethanesulfonic acid. Either the mono- or di-acid salts can be formed, and such salts can exist in either a hydrated, solvated or substantially anhydrous form. In general, the acid addition salts of these compounds are more soluble in water and various hydrophilic organic solvents, and generally demonstrate higher melting points in comparison to their free base forms. The selection criteria for the appropriate salt will be known to one skilled in the art. Other non-pharmaceutically acceptable salts e.g. oxalates may be used for example in the isolation of compounds of Formula I for laboratory use, or for subsequent conversion to a pharmaceutically acceptable acid addition salt.

A “pharmaceutically acceptable basic addition salt” is any non-toxic organic or inorganic base addition salt of the acid compounds represented by Formula I or any of its intermediates. Illustrative inorganic bases which form suitable salts include, but are not limited thereto, lithium, sodium, potassium, calcium, magnesium or barium hydroxides. Illustrative organic bases which form suitable salts include aliphatic, alicyclic or aromatic organic amines such as methylamine, trimethyl amine and picoline or ammonia. The selection of the appropriate salt may be important so that an ester functionality, if any, elsewhere in the molecule is not hydrolyzed. The selection criteria for the appropriate salt will be known to one skilled in the art.

“Solvate” means a compound of Formula I or the pharmaceutically acceptable salt of a compound of Formula I wherein molecules of a suitable solvent are incorporated in a crystal lattice. A suitable solvent is physiologically tolerable at the dosage administered as the solvate. Examples of suitable solvents, but are not limited thereto, are ethanol, water and the like. When water is the solvent, the molecule is referred to as a hydrate.

The term “stereoisomers” is a general term for all isomers of the individual molecules that differ only in the orientation of their atoms in space. It includes mirror image isomers (enantiomers), geometric (cis/trans) isomers and isomers of compounds with more than one chiral centre that are not mirror images of one another (diastereomers).

The term “treat” or “treating” means to alleviate symptoms, eliminate the causation of the symptoms either on a temporary or permanent basis, or to prevent or slow the appearance of symptoms of the named disorder or condition.

The term “therapeutically effective amount” means an amount of the compound which is effective in treating the named disorder or condition.

The term “pharmaceutically acceptable carrier” means a non-toxic solvent, dispersant, excipient, adjuvant or other material which is mixed with the active ingredient in order to permit the formation of a pharmaceutical composition, i.e., a dosage form capable of administration to the patient. One example of such a carrier is a pharmaceutically acceptable oil typically used for parenteral administration.

A “5-HT_{2C} receptor-mediated disorder”, as used herein, is a disorder in which there is believed to be involvement of the pathway controlled by the 5-HT_{2C} receptor and which is ameliorated by treatment with an agonist of the 5-HT_{2C} receptor. 5-HT_{2C} receptor-mediated disorders include a depressive disorder, an anxiety disorder, including panic attack, agoraphobia, and specific or social phobia, bipolar disorder, post-traumatic stress, an eating disorder, obesity, a gastrointestinal disorder, alcoholism, drug addiction, schizophrenia, a psychotic disorder, a sleep disorder, including sleep apnea, migraine, sexual dysfunction, a central nervous system disorder, including trauma, stroke and spinal cord injury, a cardiovascular disorder, diabetes insipidus, obsessive compulsive disorder, premenstrual tension, chronic fatigue syndrome, age-related memory disorder, personality disorder and raised intracranial pressure.

### DETAILED DESCRIPTION

Compounds of the invention conform generally to Formula I:

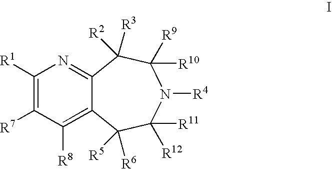

wherein R¹ to R¹² are defined hereinabove.

In an embodiment, there is provided compounds of Formula I where R⁹, R¹⁰, R¹¹ and R¹² are H:

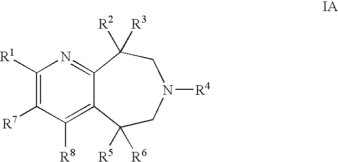

Another embodiment of the invention provides compounds of Formula I where R², R³, R⁵, R⁶, R⁹, R¹⁰, R¹¹ and R¹² are H (Formula II, below). A further embodiment of the invention provides compounds where R², R³, R⁹, R¹⁰, R¹¹ and R¹² are H (Formula III, below). Yet another embodiment of the invention provides compounds where R⁵ and R⁶, R⁹, R¹⁰, R¹¹ and R¹² are H (Formula IV, below).

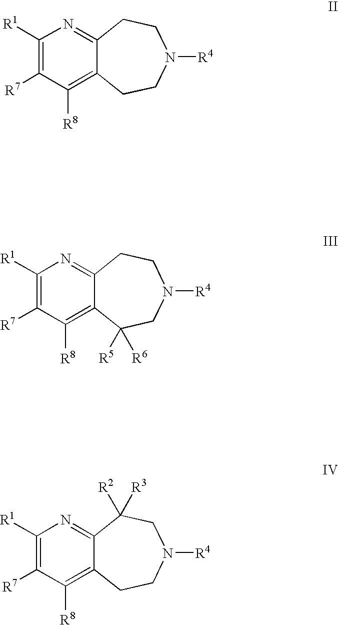

A further embodiment of the invention provides compounds of Formula I where R⁵, R⁶, R⁹, R¹⁰, R¹¹ and R¹² are H and R¹ is a 6-membered heterocyclic ring (see Formula IB, below).

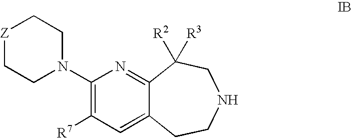

wherein:
Z is selected from CR¹⁴R¹⁵, O, NR¹⁶, C═O, S═O, SO₂ or S; and
R¹⁴ to R¹⁶ are independently selected from H, halo, hydroxy, cyano, nitro, alkyl, alkoxy, CH₂OH, haloalkyl, O-haloalkyl, hydroxyalkyl, cyanoalkyl, alkenyl, alkynyl, cycloalkyl, cycloalkenyl, heterocycloalkyl, heterocycloalkenyl, aryl, heteroaryl, alkylaryl, alkylheteroaryl, O-cycloalkyl, O-heterocycloalkyl, alkylene-O-alkyl, alkylene-O-cycloalkyl, alkylene-O-heterocycloalkyl, alkylene-O-alkylene-cycloalkyl, alkylene-O-alkylene-heterocycloalkyl, S-alkyl, S(O)-alkyl, S(O)₂-alkyl, S-cycloalkyl, S(O)-cycloalkyl, S(O)₂-cycloalkyl, S-heterocycloalkyl, S(O)-heterocycloalkyl, S(O)₂-heterocycloalkyl, O-aryl, O-heteroaryl, N(H)alkyl, N(alkyl)alkyl, N(H)-aryl, N(alkyl)-aryl, N(H)-heteroaryl, N(alkyl)-heteroaryl, alkylene-O-aryl, alkylene-O-heteroaryl, alkylene-O-alkylene-aryl, alkylene-O-alkylene-heteroaryl, S-aryl, S-heteroaryl, S(O)-aryl, S(O)-heteroaryl, S(O)₂-aryl, S(O)₂-heteroaryl, C(O)alkyl, OC(O)alkyl, C(O)Oalkyl, C(O)N(H)alkyl, C(O)N(alkyl)alkyl, S(O)₂N(H)alkyl or S(O)₂N(alkyl)alkyl.

A further embodiment of the invention provides compounds of Formula I where R⁵, R⁶, R⁹, R¹⁰, R¹¹ and R¹² are H and R¹ is a 7-membered heterocyclic ring (see Formula IC, below)

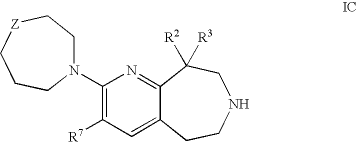

wherein:
Z is selected from CR¹⁴R¹⁵, O, NR⁶, C═O, S═O, SO₂ or S; and
R¹⁴ to R¹⁶ are independently selected from H, halo, hydroxy, cyano, nitro, alkyl, alkoxy, CH₂OH, haloalkyl, O-haloalkyl, hydroxyalkyl, cyanoalkyl, alkenyl, alkynyl, cycloalkyl, cycloalkenyl, heterocycloalkyl, heterocycloalkenyl, aryl, heteroaryl, alkylaryl, alkylheteroaryl, O-cycloalkyl, O-heterocycloalkyl, alkylene-O-alkyl, alkylene-O-cycloalkyl, alkylene-O-heterocycloalkyl, alkylene-O-alkylene-cycloalkyl, alkylene-O-alkylene-heterocycloalkyl, S-alkyl, S(O)-alkyl, S(O)₂-alkyl, S-cycloalkyl, S(O)-cycloalkyl, S(O)₂-cycloalkyl, S-heterocycloalkyl, S(O)-heterocycloalkyl, S(O)₂-heterocycloalkyl, O-aryl, O-heteroaryl, N(H)alkyl, N(alkyl)alkyl, N(H)-aryl, N(alkyl)-aryl, N(H)-heteroaryl, N(alkyl)-heteroaryl, alkylene-O-aryl, alkylene-O-heteroaryl, alkylene-O-alkylene-aryl, alkylene-O-alkylene-heteroaryl, S-aryl, S-heteroaryl, S(O)-aryl, S(O)-heteroaryl, S(O)₂-aryl, S(O)₂-heteroaryl, C(O)alkyl, OC(O)alkyl, C(O)Oalkyl, C(O)N(H)alkyl, C(O)N(alkyl)alkyl, S(O)₂N(H)alkyl or S(O)₂N(alkyl)alkyl.

It will be understood by those of skill in the art that when compounds of the present invention contain one or more chiral centers, the compounds of the invention may exist in, and be isolated as, enantiomeric or diastereomeric forms, or as a racemic mixture. The present invention includes any possible enantiomers, diastereomers, racemates or mixtures thereof, of a compound of Formula I. The optically active forms of the compound of the invention may be prepared, for example, by chiral chromatographic separation of a racemate or chemical or enzymatic resolution methodology, by synthesis from optically active starting materials or by asymmetric synthesis based on the procedures described thereafter.

It will also be appreciated by those of skill in the art that certain compounds of the present invention may exist as geometrical isomers, for example E and Z isomers of alkenes. The present invention includes any geometrical isomer of a compound of Formula I. It will further be understood that the present invention encompasses tautomers of the compounds of Formula I.

It will also be understood by those of skill in the art that certain compounds of the present invention may exist in solvated, for example hydrated, as well as unsolvated forms. It will further be understood that the present invention encompasses all such solvated forms of the compounds of Formula I.

Within the scope of the invention are also salts of the compounds of Formula I. Generally, pharmaceutically acceptable salts of compounds of the present invention are obtained using standard procedures well known in the art, for example, by reacting a sufficiently basic compound, for example an alkyl amine with a suitable acid, for example, HCl or acetic acid, to afford a salt with a physiologically acceptable anion. It is also possible to make a corresponding alkali metal (such as sodium, potassium, or lithium) or an alkaline earth metal (such as a calcium) salt by treating a compound of the present invention having a suitably acidic proton, such as a carboxylic acid or a phenol, with one equivalent of an alkali metal or alkaline earth metal hydroxide or alkoxide (such as the ethoxide or methoxide), or a suitably basic organic amine (such as choline or meglumine) in an aqueous medium, followed by conventional purification techniques. Additionally, quaternary ammonium salts can be prepared by the addition of alkylating agents, for example, to neutral amines.

In one embodiment of the present invention, the compound of Formula I may be converted to a pharmaceutically acceptable salt or solvate thereof, particularly, an acid addition salt such as a hydrochloride, hydrobromide, phosphate, acetate, fumarate, maleate, tartrate, citrate, methanesulphonate or p-toluenesulphonate.

Specific examples of the present invention include the following compounds, their pharmaceutically acceptable salts, hydrates, solvates, optical isomers, and combinations thereof:
(9R)-2-(4,4-difluoropiperidin-1-yl)-9-methyl-6,7,8,9-tetrahydro-5H-pyrido[2,3d]azepine;
(9R)-2-(4-fluoropiperidin-1-yl)-9-methyl-6,7,8,9-tetrahydro-5H-pyrido[2,3-d]azepine;
(9R)-9-methyl-2-(1,4-oxazepan-4-yl)-6,7,8,9-tetrahydro-5H-pyrido[2,3-d]azepine;
(9R)-9-methyl-2-morpholin-4-yl-6,7,8,9-tetrahydro-5H-pyrido[2,3-d]azepine;
(9R)-9-methyl-2-piperidin-1-yl-6,7,8,9-tetrahydro-5H-pyrido[2,3-d]azepine;
(9R)-N-ethyl-N,9-dimethyl-6,7,8,9-tetrahydro-5H-pyrido[2,3-d]azepin-2-amine;
(9S)-2-(4,4-difluoropiperidin-1-yl)-9-methyl-6,7,8,9-tetrahydro-5H-pyrido[2,3-d]azepine;
(9S)-2-(4-fluoropiperidin-1-yl)-9-methyl-6,7,8,9-tetrahydro-5H-pyrido[2,3-d]azepine;
(9S)-9-methyl-2-(1,4-oxazepan-4-yl)-6,7,8,9-tetrahydro-5H-pyrido[2,3-d]azepine
(9S)-9-methyl-2-morpholin-4-yl-6,7,8,9-tetrahydro-5H-pyrido[2,3-d]azepine;
(9S)-9-methyl-2-piperidin-1-yl-6,7,8,9-tetrahydro-5H-pyrido[2,3-d]azepine;
(9S)-N-ethyl-N,9-dimethyl-6,7,8,9-tetrahydro-5H-pyrido[2,3-d]azepin-2-amine;
2-(1,4-diazepan-1-yl)-6,7,8,9-tetrahydro-5H-pyrido[2,3-d]azepine;
2-(1,4-oxazepan-4-yl)-6,7,8,9-tetrahydro-5H-pyrido[2,3-d]azepine;
2-(1-oxidothiomorpholin-4-yl)-6,7,8,9-tetrahydro-5H-pyrido[2,3-d]azepine;
2-(3-thienyl)-6,7,8,9-tetrahydro-5H-pyrido[2,3-d]azepine;
2-(4,4-difluoroazepan-1-yl)-6,7,8,9-tetrahydro-5H-pyrido[2,3-d]azepine;
2-(4,4-difluoroazepan-1-yl)-9-methyl-6,7,8,9-tetrahydro-5H-pyrido[2,3-d]azepine;
2-(4,4-difluoropiperidin-1-yl)-6,7,8,9-tetrahydro-5H-pyrido[2,3-d]azepine;
2-(4,4-difluoropiperidin-1-yl)-9-methyl-6,7,8,9-tetrahydro-5H-pyrido[2,3-d]azepine;
2-(4-fluoropiperidin-1-yl)-6,7,8,9-tetrahydro-5H-pyrido[2,3-d]azepine;
2-(4-fluoropiperidin-1-yl)-9-methyl-6,7,8,9-tetrahydro-5H-pyrido[2,3-d]azepine;
2-(4-methylpiperazin-1-yl)-6,7,8,9-tetrahydro-5H-pyrido[2,3-d]azepine;
2-(8-azabicyclo[3.2.1]oct-8-yl)-6,7,8,9-tetrahydro-5H-pyrido[2,3-d]azepine;
2-(trifluoromethyl)-6,7,8,9-tetrahydro-5H-pyrido[2,3-d]azepine;
2,9-dimethyl-6,7,8,9-tetrahydro-5H-pyrido[2,3-d]azepine;
2-[methyl(6,7,8,9-tetrahydro-5H-pyrido[2,3-d]azepin-2-yl)amino]ethanol;
2-azepan-1-yl-6,7,8,9-tetrahydro-5H-pyrido[2,3-d]azepine;
2-azepan-1-yl-9-isopropyl-6,7,8,9-tetrahydro-5H-pyrido[2,3-d]azepine;
2-azepan-1-yl-9-methyl-6,7,8,9-tetrahydro-5H-pyrido[2,3-d]azepine;
2-cyclopropyl-6,7,8,9-tetrahydro-5H-pyrido[2,3-d]azepine;
2-cyclopropyl-9-methyl-6,7,8,9-tetrahydro-5H-pyrido[2,3-d]azepine;
2-isopropenyl-6,7,8,9-tetrahydro-5H-pyrido[2,3-d]azepine;
2-isopropenyl-9-methyl-6,7,8,9-tetrahydro-5H-pyrido[2,3-d]azepine;
2-isopropyl-6,7,8,9-tetrahydro-5H-pyrido[2,3-d]azepine;
2-isopropyl-9-methyl-6,7,8,9-tetrahydro-5H-pyrido[2,3-d]azepine;
2-methoxy-6,7,8,9-tetrahydro-5H-pyrido[2,3-d]azepine;
2-methoxy-9-methyl-6,7,8,9-tetrahydro-5H-pyrido[2,3-d]azepine;
2-methyl-6,7,8,9-tetrahydro-5H-pyrido[2,3-d]azepine;
2-morpholin-4-yl-6,7,8,9-tetrahydro-5H-pyrido[2,3-d]azepine;
2-piperazin-1-yl-6,7,8,9-tetrahydro-5H-pyrido[2,3-d]azepine;
2-piperidin-1-yl-6,7,8,9-tetrahydro-5H-pyrido[2,3-d]azepine;
2-pyrrolidin-1-yl-6,7,8,9-tetrahydro-5H-pyrido[2,3-d]azepine;
2-tert-butyl-6,7,8,9-tetrahydro-5H-pyrido[2,3-d]azepine;
2-thiomorpholin-4-yl-6,7,8,9-tetrahydro-5H-pyrido[2,3-d]azepine;
2-vinyl-6,7,8,9-tetrahydro-5H-pyrido[2,3-d]azepine;
3-bromo-2-methoxy-6,7,8,9-tetrahydro-5H-pyrido[2,3-d]azepine;
3-bromo-2-morpholin-4-yl-6,7,8,9-tetrahydro-5H-pyrido[2,3-d]azepine;
3-bromo-2-piperidin-1-yl-6,7,8,9-tetrahydro-5H-pyrido[2,3-d]azepine;
3-bromo-N-ethyl-N-methyl-6,7,8,9-tetrahydro-5H-pyrido[2,3-d]azepin-2-amine;
3-chloro-2-(1,4-oxazepan-4-yl)-6,7,8,9-tetrahydro-5H-pyrido[2,3-d]azepine;
3-chloro-2-(4,4-difluoropiperidin-1-yl)-6,7,8,9-tetrahydro-5H-pyrido[2,3-d]azepine;
3-chloro-2-(4-fluoropiperidin-1-yl)-6,7,8,9-tetrahydro-5H-pyrido[2,3-d]azepine;
3-chloro-2-morpholin-4-yl-6,7,8,9-tetrahydro-5H-pyrido[2,3-d]azepine;
3-chloro-2-piperidin-1-yl-6,7,8,9-tetrahydro-5H-pyrido[2,3-d]azepine;
6,7,8,9-tetrahydro-5H-pyrido[2,3-d]azepine-2-carbaldehyde;
6,7,8,9-tetrahydro-5H-pyrido[2,3-d]azepine-2-carbonitrile;
9-ethyl-2-morpholin-4-yl-6,7,8,9-tetrahydro-5H-pyrido[2,3-d]azepine;
9-ethyl-2-piperidin-1-yl-6,7,8,9-tetrahydro-5H-pyrido[2,3-d]azepine;
9-isopropyl-2-morpholin-4-yl-6,7,8,9-tetrahydro-5H-pyrido[2,3-d]azepine;
9-isopropyl-2-piperidin-1-yl-6,7,8,9-tetrahydro-5H-pyrido[2,3-d]azepine;
9-methyl-2-(1,4-oxazepan-4-yl)-6,7,8,9-tetrahydro-5H-pyrido[2,3-d]azepine;
9-methyl-2-morpholin-4-yl-6,7,8,9-tetrahydro-5H-pyrido[2,3-d]azepine;
9-methyl-2-piperidin-1-yl-6,7,8,9-tetrahydro-5H-pyrido[2,3-d]azepine;
ethyl 4-(6,7,8,9-tetrahydro-5H-pyrido[2,3-d]azepin-2-yl)piperazine-1-carboxylate;
N,9-diethyl-N-methyl-6,7,8,9-tetrahydro-5H-pyrido[2,3-d]azepin-2-amine;
N,N,9-trimethyl-6,7,8,9-tetrahydro-5H-pyrido[2,3-d]azepin-2-amine;
N,N-diallyl-6,7,8,9-tetrahydro-5H-pyrido[2,3-d]azepin-2-amine;
N,N-diethyl-6,7,8,9-tetrahydro-5H-pyrido[2,3-d]azepin-2-amine;
N,N-dimethyl-6,7,8,9-tetrahydro-5H-pyrido[2,3-d]azepin-2-amine;
N,N-dimethyl-6,7,8,9-tetrahydro-5H-pyrido[2,3-d]azepine-2-carboxamide;
N-benzyl-N-methyl-6,7,8,9-tetrahydro-5H-pyrido[2,3-d]azepin-2-amine;
N-ethyl-N,9-dimethyl-6,7,8,9-tetrahydro-5H-pyrido[2,3-d]azepin-2-amine;
N-ethyl-N-methyl-6,7,8,9-tetrahydro-5H-pyrido[2,3-d]azepin-2-amine;
N-isopropyl-N-methyl-6,7,8,9-tetrahydro-5H-pyrido[2,3-d]azepin-2-amine;
N-methyl-6,7,8,9-tetrahydro-5H-pyrido[2,3-d]azepin-2-amine;
N-methyl-N-(6,7,8,9-tetrahydro-5H-pyrido[2,3-d]azepin-2-yl)acetamide;
2-Phenyl-6,7,8,9-tetrahydro-5H-pyrido[2,3-d]azepine;
6,7,8,9-Tetrahydro-5H-pyrido[2,3-d]azepine-2-carbonitrile;
2-Chloro-6,7,8,9-tetrahydro-5H-pyrido[2,3-d]azepine;
[2-(6-Methoxy-3-methyl-pyridin-2-yl)-ethyl]-methyl-amine; and/or
7,8,9-Tetrahydro-5H-pyrido[2,3-d]azepin-2-ol.

Several methods for preparing compounds of this invention are illustrated in the following, non-limiting, Schemes and Examples. Starting materials and the requisite intermediates are in some cases commercially available, or can be prepared according to literature procedures or as illustrated herein.

Compounds of Formula I, wherein R¹¹ and R¹² are H, may be prepared as follows:

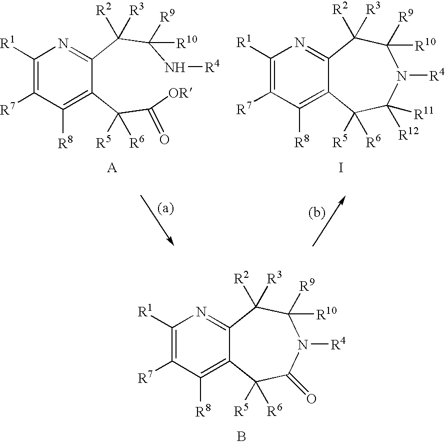

whereby R′ can be alkyl or cycloalkyl and (a) comprises heat and base assisted cyclization of a compound of Formula A to provide a compound of Formula B and (b) comprises reduction of the carbonyl of the amide of the compound of Formula B. For example, (a) comprises heating in DMF and (b) comprises reduction with LiAlH₄/AlCl₃.

Compounds of Formula I, wherein R⁹ and R¹⁰ are H, may be prepared as follows:

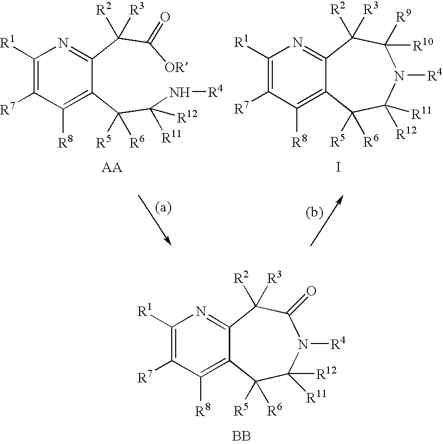

whereby R′ can be alkyl or cycloalkyl and (a) comprises heat and base assisted cyclization of a compound of Formula AA to provide a compound of Formula BB and (b) comprises reduction of the carbonyl of the amide of the compound of Formula B. For example, (a) comprises heating in DMF and (b) comprises reduction with LiAlH₄/AlCl₃.

Generally, compounds of Formula I, wherein R¹¹ and R¹² are H, may be prepared via reduction of a carbonyl of the following amide:

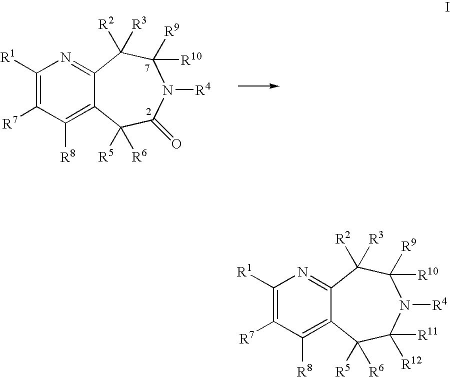

Reduction can occur, for example, using LiAlH₄/AlCl₃. Compounds of Formula I, wherein R⁹ and R¹⁰ are H and R¹¹ and R¹² are not H, may be prepared via reduction of a carbonyl group of a similar amide except that the carbonyl is now C7 instead of C2, and C2 is substituted with R¹¹ and R¹².

Generally, compounds of Formula I, wherein R⁹, R¹⁰, R¹¹ and R¹² are H, may be prepared via reduction of the carbonyl groups of the following amide:

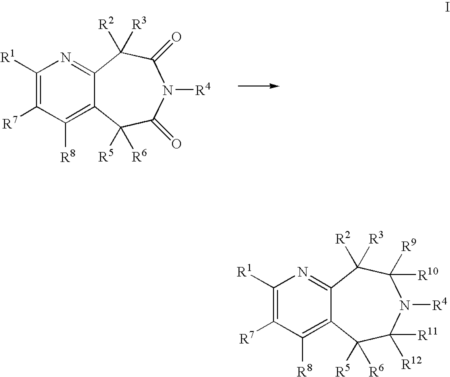

Again, reduction can occur, for example, using LiAlH₄/AlCl₃.

Compounds of Formula I, wherein R¹¹ and R¹² are H, may be prepared as follows:

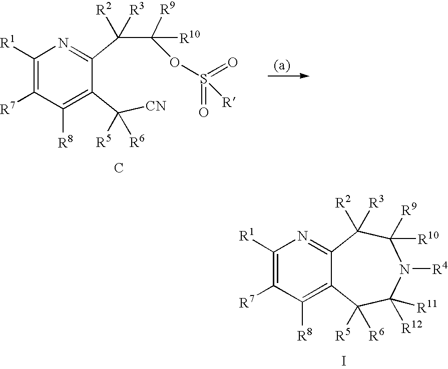

whereby (a) comprises selective cyano reduction followed by cyclization of the compound of Formula C. R′ can be alkyl or cycloalkyl. For example, cyano reduction using LiAlH₄/AlCl₃ and DIPEA in acetonitrile for cyclization. The resultant Formula I can be converted to a salt addition of an acid, for example.

Compounds of Formula I, wherein R¹¹ and R¹² are H, may be prepared as follows:

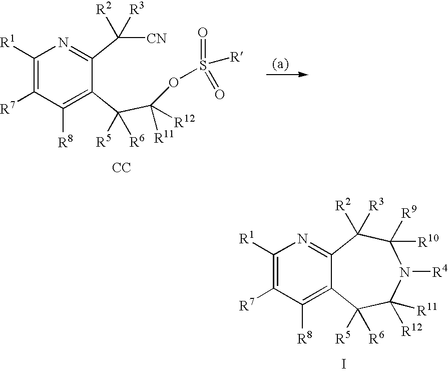

whereby (a) comprises selective cyano reduction followed by cyclization of the compound of Formula CC. R′ can be alkyl or cycloalkyl. For example, cyano reduction using LiAlH₄/AlCl₃ and DIPEA in acetonitrile for cyclization. The resultant Formula I can be converted to a salt addition of an acid, for example.

Compounds of Formula I may also be prepared as follows:

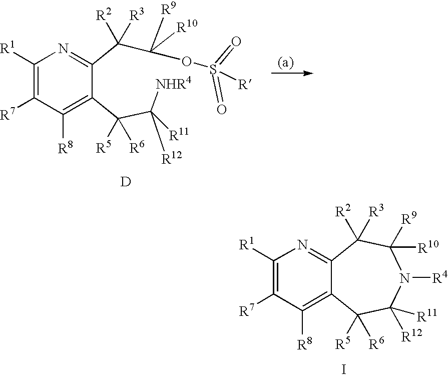

whereby (a) comprises cyclization of the compound of Formula D, whereby R′ can be alkyl or cycloalkyl. For example, base can be used to initiate cyclization.

Compounds of Formula I may also be prepared as follows:

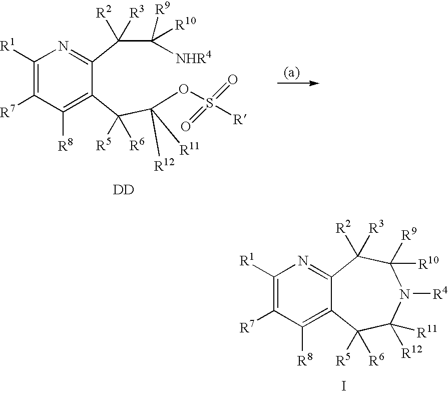

whereby (a) comprises cyclization of the compound of Formula DD, whereby R′ can be alkyl or cycloalkyl. For example, base can be used to initiate cyclization.

In specific embodiments, the compounds of Formula II where R⁴ is H or alkyl, R⁷ and R⁸ are H and R¹ is methoxy may be prepared according to Scheme 1, below, from nitrile intermediate V by alkylation with benzylamine, followed by hydrolysis of the nitrile function to afford the amino ester VII. Microwave-assisted cyclization to azepinone VIII and reduction with LiAlH₄/AlCl₃ gave the benzyl-protected azepine IX. Subsequent hydrogenolysis of the protecting group provided the compound of Formula II. The intermediate V was prepared from 2-(2-chloroethyl)-3-(chloromethyl)-6-methoxypyridine [Feng, S.; He, X.; Yu, G.; Yu, X.; Bai, D. Org. Prep. Proced. Int. 2004, 36 (2); 129-134] via mono-cyanation and the Finkelstien chloro-iodo exchange.

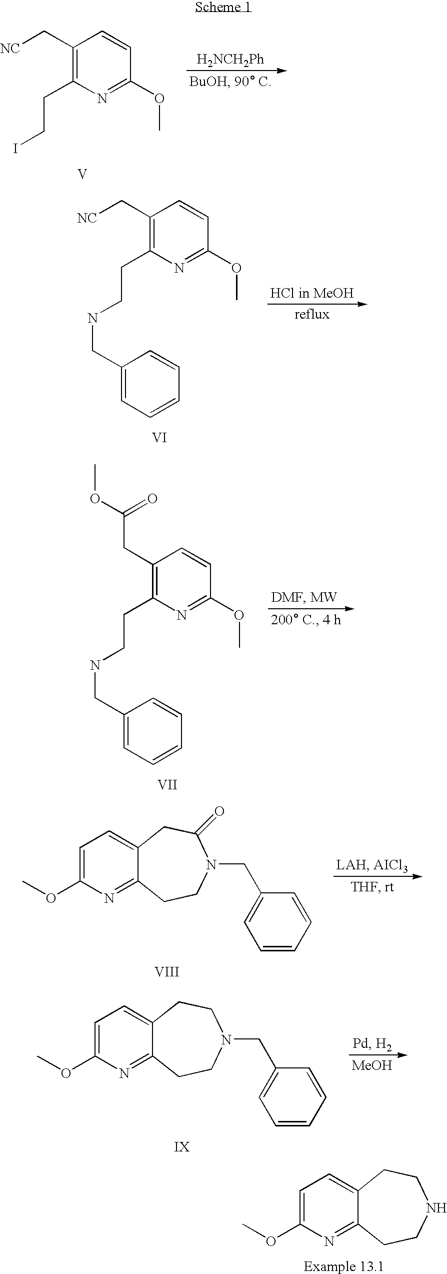

Alternatively, compounds of Formula II can also be prepared from the readily accessible 2-substituted-3-iodopyridine XI [X=Cl, Br or OTf, [Zhang, Y et al., J. Med. Chem. 2004, 47, 2453] which is in turn obtained from the 2-Hydroxypyridine X. α-Arylation of esters with XI under metal-catalyzed coupling conditions [Hartwig et al., J. Med. Chem. 2004, 47, 2453], or simple α-arylation of a dialkylmalonate [Buchwald et al, Org. Lett. 2002, 4, 269] followed by decarboxylation gives XII. Coupling of XII with acrylates (eg. t-butyl acrylate) under standard Heck conditions gives XII. Catalytic hydrogenation followed by ester hydrolysis delivers acids XIV (R⁸=H). Curtis rearrangement and cyclization afford XVI (M=H, H). Subsequent reduction then provides compounds of Formula II.

Another alternative to compounds of Formula II includes the transformation of intermediate XI, simultaneously or sequentially, into the diester intermediate XVIII. Cyclization to form the imide XVI (M=O), followed by reduction, provides compounds of Formula II.

Compounds of Formula II can also be obtained from diester XVIII by reduction first to the diol XIX. Activation of the diol (eg mesylation or halide formation) and cyclization with amines give II (Scheme 2).

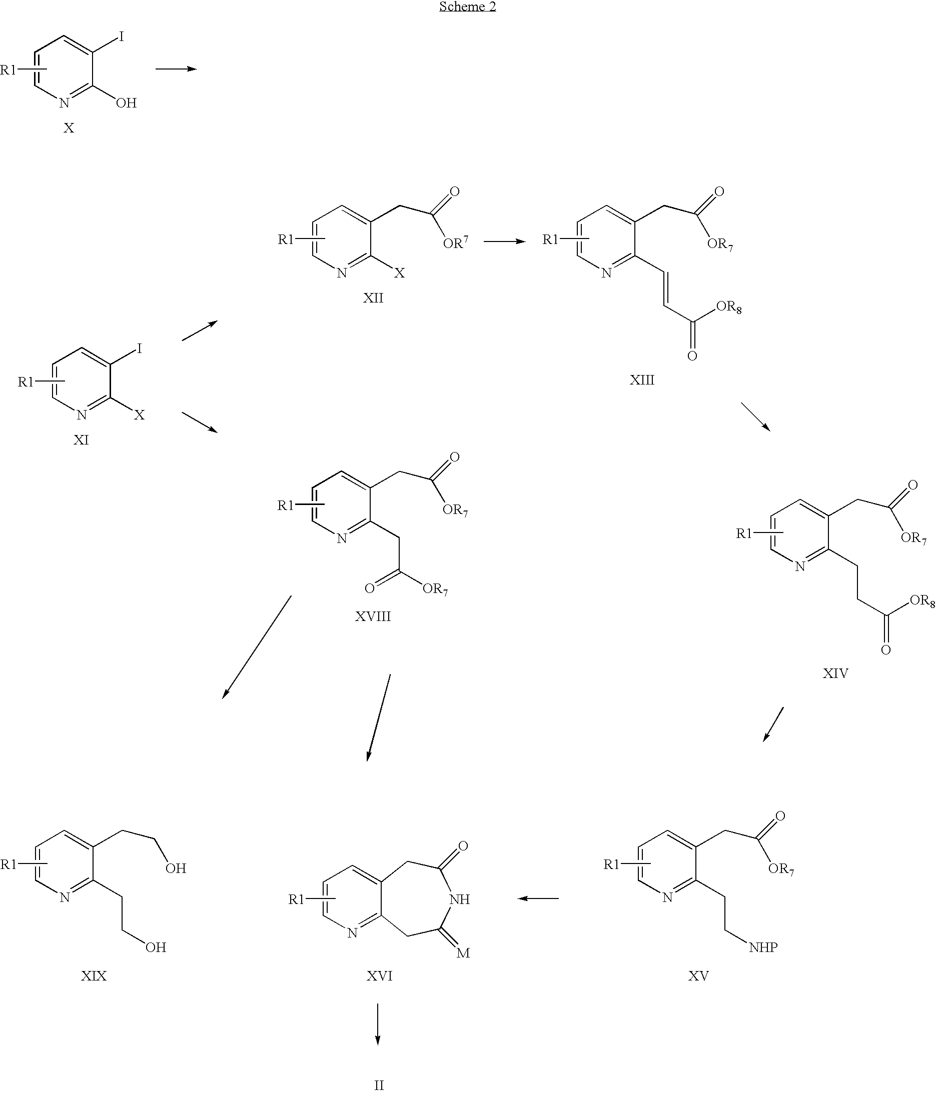

Compounds of Formula III where R⁴ is H or alkyl and R⁵ and/or R⁶ is H or alkyl respectively can be prepared in a manner similar to that shown in Scheme 2 (see Scheme 3). Alternatively, α-alkyations of XII also provide intermediates such as XX, which may then be transformed into compounds of Formula III (Scheme 4).

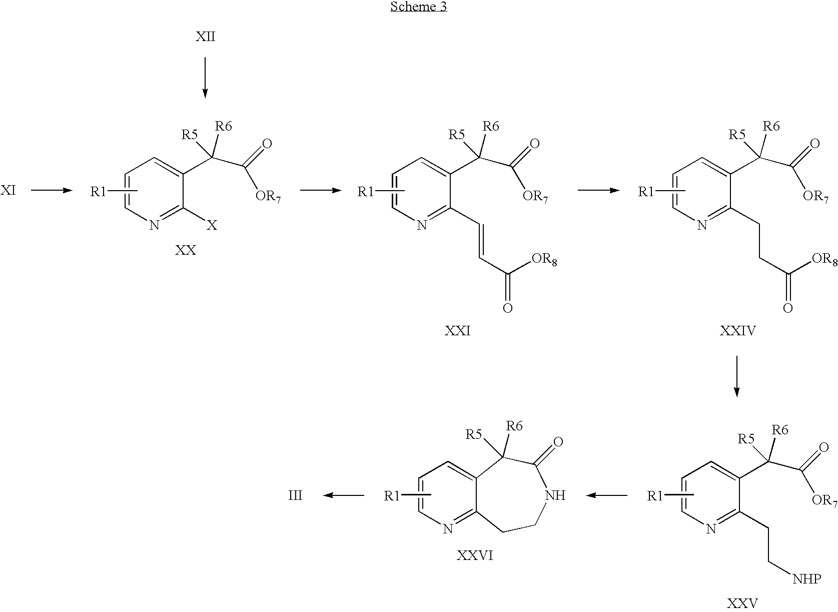

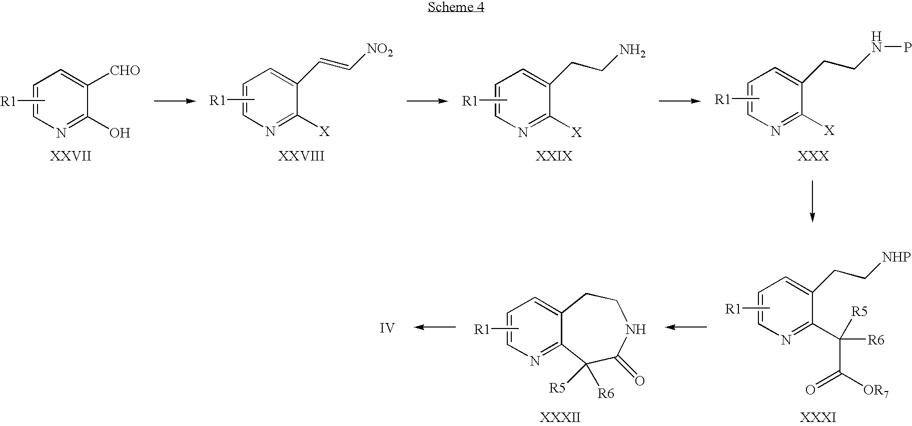

Compounds of Formula IV can be prepared from pyridyl carboxaldehydes such as XXVII (X=Cl, Br, I, OTf) by condensation with nitromethane followed by reduction to the amine XXIX. Protection of the amine (e.g. Boc-protection) followed by simple α-arylation of esters as above gives XXXI which, on de-protection and cyclization, affords azepinone XXXII. Reduction with LiAlH₄ or other reducing agents such as Red-Al gives the compounds of Formula IV.

Alternatively, compounds of Formula IV can also be obtained from the intermediate XXXIII [Feng, S.; He, X.; Yu, G.; Yu, X.; Bai, D. Org. Prep. Proced. Int. 2004, 36 (2); 129-134] by simple alkylation to give XXXIV which is then transformed to the desired compounds in a manner similar to that shown in Scheme 1 (see Scheme 5).

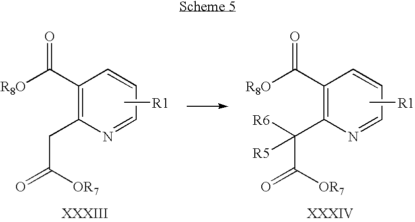

For another synthetic strategy to prepare compounds of Formula III wherein R⁷=H and R⁸=H, R⁵ and R⁶ is H (as in Formula II) and R¹ is an amine containing group is outlined in Scheme 6. Condensation of the β-ketodiester B with the acetylenic amide A gives the functionalized pyridone C which on treatment with Ag₂CO₃ and methyl iodide gives the methoxypyridine intermediate D. The intermediate D can be treated with a number of alkylating groups (e.g. R⁵ or R⁶=Methyl is shown) to introduce functionality onto the azepine ring. Treatment of the diester D with a reducing agent (e.g. LiAlH₄) provides diol intermediate E. Treatment of diol E with mesyl chloride gives the chloro-mesylate F which on mild cyanation with NaCN in DMSO gives the cyano-mesylate G. Selective cyano reduction (e.g. with alane generated in situ from AlCl₃ and LiAlH₄) followed by cyclization gives the pyridyl-fused azepine intermediate H. O-Deprotection with HBr in acetic acid to I followed by N-protection with for example (BOC)₂O affords intermediate J. Triflation of J to the versatile intermediate K followed by coupling with the various amines give the Boc-protected precusor L of the compounds of Formula III. Treatment of L with HCl gives M, the HCl salts of the compounds of this invention.

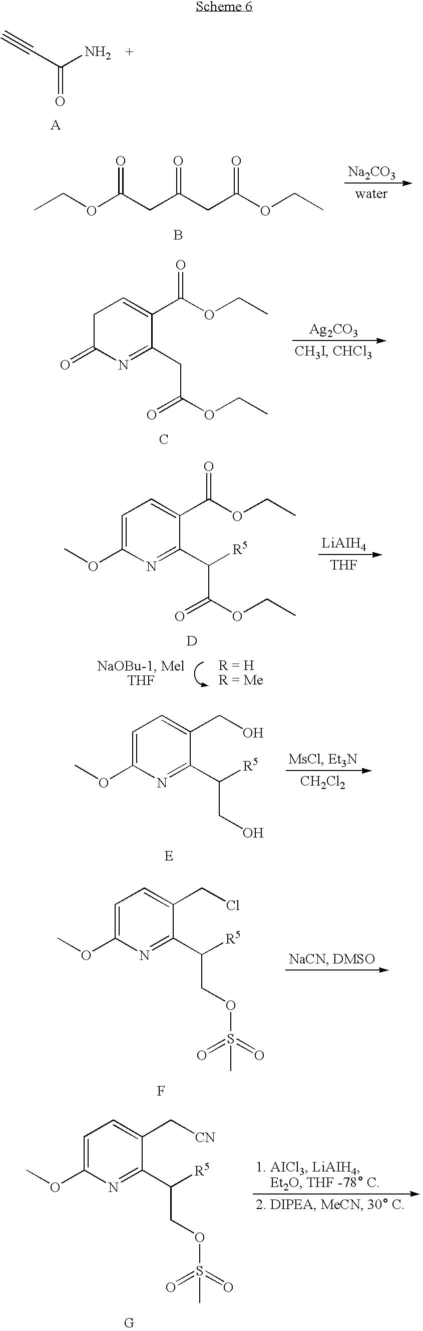

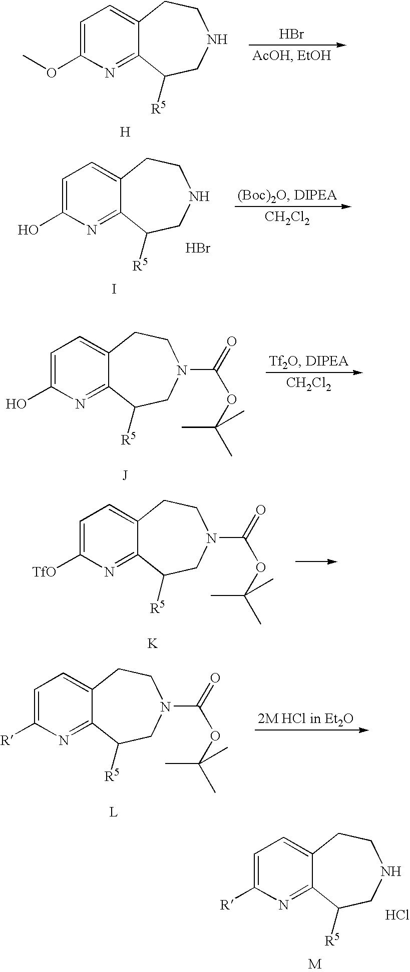

The compounds of Formula II where R¹=CF₃ were prepared according to Scheme 7. The commercially available methyl acid is treated with base to effect methyl-deprotonation. Subsequent reduction with alane provides the common diol intermediate that is further transformed into the compounds of this invention in a manner analogous to Scheme 6.

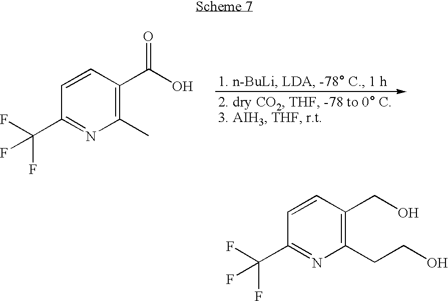

Acid addition salts of the compounds of Formula I are most suitably formed from pharmaceutically acceptable acids, and include for example those formed with inorganic acids e.g. hydrochloric, sulphuric or phosphoric acids and organic acids e.g. succinic, maleic, acetic or fumaric acid. Other non-pharmaceutically acceptable salts e.g. oxalates may be used for example in the isolation of compounds of Formula I for laboratory use, or for subsequent conversion to a pharmaceutically acceptable acid addition salt. Also included within the scope of the invention are base addition salts (such as sodium, potassium and ammonium salts), solvates and hydrates of compounds of the invention.

The conversion of a given compound salt to a desired compound salt is achieved by applying standard techniques, well known to one skilled in the art.

Compounds in accordance with the invention have been shown to be potent agonists or partial agonists of the 5-HT_{2C} receptor and have good specificity for the 5-HT_{2C} receptor compared to the 5-HT_{2A} and 5-HT_{2B} receptors.

In embodiments of the invention, the compounds of the invention have an EC₅₀ value for the human 5-HT_{2c} receptor less than 1000 nM, or less than 500 nM, or less than 300 nM, or less than 100 nM.

The compounds of the invention are therefore of interest for the treatment of 5-HT_{2c} receptor-mediated disorders, including a depressive disorder, an anxiety disorder, including panic attack, agoraphobia, and specific or social phobia, bipolar disorder, post-traumatic stress, an eating disorder, obesity, a gastrointestinal disorder, alcoholism, drug addiction, schizophrenia, a psychotic disorder, a sleep disorder, including sleep apnea, migraine, sexual dysfunction, a central nervous system disorder, including trauma, stroke and spinal cord injury, a cardio-vascular disorder, diabetes insipidus, obsessive compulsive disorder, premenstrual tension, chronic fatigue syndrome, age-related memory disorder, personality disorder and raised intracranial pressure.

The studies described herein of the effect of compounds of the invention in a feeding assay which is an accepted in vivo rodent model for studies on overeating and control of food intake confirms the potential of these compounds for treatment of obesity.

Compounds of the invention have also been shown to be effective in inhibiting locomotor activity in a rodent model relevant to treatment of schizophrenia or other psychotic disorders.

For pharmaceutical use, the compounds of the invention are, for instance, administered orally, sublingually, rectally, nasally, vaginally, topically (including the use of a patch or other transdermal delivery device), by pulmonary route by use of an aerosol, or parenterally, including, for example, intramuscularly, subcutaneously, intraperitoneally, intraarterially, intravenously or intrathecally. Administration can be by means of a pump for periodic or continuous delivery. The compounds of the invention are administered alone, or are combined with a pharmaceutically-acceptable carrier or excipient according to standard pharmaceutical practice. For the oral mode of administration, the compounds of the invention are used in the form of tablets, capsules, lozenges, chewing gum, troches, powders, syrups, elixirs, aqueous solutions and suspensions, and the like. In the case of tablets, carriers that are used include lactose, sodium citrate and salts of phosphoric acid. Various disintegrants such as starch, and lubricating agents such as magnesium stearate and talc, are commonly used in tablets. For oral administration in capsule form, useful diluents are lactose and high molecular weight polyethylene glycols. If desired, certain sweetening and/or flavoring agents are added. For parenteral administration, sterile solutions of the compounds of the invention are usually prepared, and the pHs of the solutions are suitably adjusted and buffered. For intravenous use, the total concentration of solutes should be controlled to render the preparation isotonic. For ocular administration, ointments or droppable liquids may be delivered by ocular delivery systems known to the art such as applicators or eye droppers. Such compositions can include mucomimetics such as hyaluronic acid, chondroitin sulfate, hydroxypropyl methylcellulose or polyvinyl alcohol, preservatives such as sorbic acid, EDTA or benzylchromium chloride, and the usual quantities of diluents and/or carriers. For pulmonary administration, diluents and/or carriers will be selected to be appropriate to allow the formation of an aerosol.

Suppository forms of the compounds of the invention are useful for vaginal, urethral and rectal administrations. Such suppositories will generally be constructed of a mixture of substances that is solid at room temperature but melts at body temperature. The substances commonly used to create such vehicles include theobroma oil, glycerinated gelatin, hydrogenated vegetable oils, mixtures of polyethylene glycols of various molecular weight and fatty acid esters of polyethylene glycol. See, Remington's Pharmaceutical Sciences, 16th Ed., Mack Publishing, Easton, Pa., 1980, pp. 1530-1533 for further discussion of suppository dosage forms. Analogous gels or creams can be used for vaginal, urethral and rectal administrations.

Numerous administration vehicles will be apparent to those of ordinary skill in the art, including without limitation slow release formulations, liposomal formulations and polymeric matrices.

Examples of pharmaceutically acceptable acid addition salts for use in the present invention include those derived from mineral acids, such as hydrochloric, hydrobromic, phosphoric, metaphosphoric, nitric and sulfuric acids, and organic acids, such as tartaric, acetic, citric, malic, lactic, fumaric, benzoic, glycolic, gluconic, succinic, p-toluenesulphonic and arylsulphonic acids, for example. Examples of pharmaceutically acceptable base addition salts for use in the present invention include those derived from non-toxic metals such as sodium or potassium, ammonium salts and organoamino salts such as triethylamine salts. Numerous appropriate such salts will be known to those of ordinary skill.

The physician or other health care professional can select the appropriate dose and treatment regimen based on the subject's weight, age, and physical condition. Dosages will generally be selected to maintain a serum level of compounds of the invention between about 0.01 μg/cc and about 1000 μg/cc, preferably between about 0.1 μg/cc and about 100 μg/cc. For parenteral administration, an alternative measure of preferred amount is from about 0.001 mg/kg to about 10 mg/kg (alternatively, from about 0.01 mg/kg to about 10 mg/kg), more preferably from about 0.01 mg/kg to about 1 mg/kg (from about 0.1 mg/kg to about 1 mg/kg), will be administered. For oral administrations, an alternative measure of preferred administration amount is from about 0.001 mg/kg to about 10 mg/kg (from about 0.1 mg/kg to about 10 mg/kg), more preferably from about 0.01 mg/kg to about 1 mg/kg (from about 0.1 mg/kg to about 1 mg/kg). For administrations in suppository form, an alternative measure of preferred administration amount is from about 0.1 mg/kg to about 10 mg/kg, more preferably from about 0.1 mg/kg to about 1 mg/kg.

When introducing elements disclosed herein, the articles “a”, “an”, “the”, and “said” are intended to mean that there are one or more of the elements. The terms “comprising”, “having”, “including” are intended to be open-ended and mean that there may be additional elements other than the listed elements.

The above disclosure generally describes the present invention. A more complete understanding can be obtained by reference to the following specific Examples. These Examples are described solely for purposes of illustration and are not intended to limit the scope of the invention. Changes in form and substitution of equivalents are contemplated as circumstances may suggest or render expedient. Although specific terms have been employed herein, such terms are intended in a descriptive sense and not for purposes of limitation.

### EXAMPLES

### Example 1.1

### Propiolamide

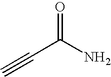

Methyl propioate (5 mL, 55 mmol) was added to ammonium hydroxide (15 mL) at −50 to −60° C. over 20 minutes. The reaction mixture was stirred at this temperature for 1 hour. The solvent was evaporated and the residue dried under vacuum to give the product (3.7 g, 92%). ¹H NMR (300 MHz, CDCl₃): δ (ppm) 6.35 (bs, 1H), 5.97 (bs, 1H), 2.88 (s, 1H).

### Example 2.1

### Ethyl 2-(2-ethoxy-2-oxoethyl)-6-oxo-1,6-dihydropyridine-3-carboxylate

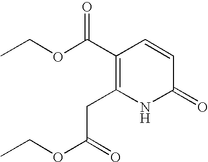

Propiolamide (20.0 g, 289.6 mmol), diethyl 3-oxopentanedioate (87.8 g, 434.4 mmol) and sodium carbonate (24.6 g, 231.7 mmol) were mixed in water (800 mL) at 0° C. and then warmed to r.t. over 4 hours. The reaction was allowed to continue to stir at r.t. for 3 days. The reaction was neutralized with aqueous hydrochloric acid (5M) at 0° C. with vigorous stirring. A solid precipitate was collected by filtration and washed with diethyl ether/hexanes (2:1) to yield a first batch of the title compound (43 g). The filtrate was further extracted with ethyl acetate and the combined organic phases were dried over sodium sulphate and purified by column chromatography (10-80% ethyl acetate/hexanes) to yield another batch of the title compound (7 g), in total gave 50 g (68%). ¹H NMR (300 MHz, CDCl₃): δ(ppm) 13.12 (brs, 1H), 8.08 (d, 1H), 6.52 (d, 1H), 4.3 (q, 2H), 4.21 (q, 2H), 4.13 (s, 2H), 1.29 (m, 6H).

### Example 3.1

### Ethyl 2-(2-ethoxy-2-oxoethyl)-6-methoxynicotinate

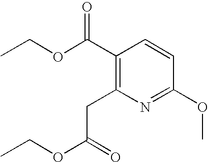

The title compound from Example 2.1 (20 g, 79.05 mmol) was stirred with silver carbonate (23.7 g, 105.3 mmol) and iodomethane (40.7 g, 286.6 mmol) in chloroform (180 mL) at 50° C. overnight. The reaction mixture was filtered and the filtrate was concentrated to give the crude title compound (22 g, quantitative). ¹H NMR (300 MHz, CDCl₃): δ (ppm) 8.21 (d, 1H), 6.69 (d, 1H), 4.32 (q, 2H), 4.17 (m, 4H), 3.97 (s, 3H), 1.37 (t, 3H), 1.29 (t, 3H).

### Example 3.2

### Ethyl 2-(2-ethoxy-1-methyl-2-oxoethyl)-6-methoxynicotinate

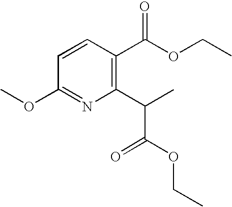

To a suspension of sodium tert-butoxide (7.98 g, 82.3 mmol) in tetrahydrofuran (250 mL) was added a solution of ethyl 2-(2-ethoxy-2-oxoethyl)-6-methoxynicotinate (20.0 g, 74.8 mmol) (the title compound from Example 3.1) in tetrahydrofuran (100 mL) at 0° C. over 15 minutes. After being stirred for 15 minutes, iodomethane (53.1 g, 374 mmol) was added at 0° C. The reaction was allowed to warm up to r.t. over 2 hours. Acetic acid (1 mL) was added at 0° C. and the mixture was stirred for 30 minutes. The reaction was diluted with water and extracted with ethyl acetate. The organic layer was dried over magnesium sulfate, filtered and concentrated to give the title compound (20.16 g, 96%). ¹H NMR (300 MHz, CDCl₃): δ(ppm) 8.16 (d, 1H), 6.64 (d, 1H), 4.87 (q, 1H), 4.33 (m, 2H), 4.13 (m, 2H), 3.94 (3H), 1.54 (d, 3H), 1.36 (m, 3H), 1.18 (m, 3H).

### Example 4.1

### 2-(3-Hydroxymethyl-6-methoxy-pyridin-2-yl)-ethanol

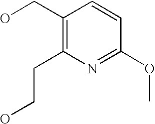

To a suspension of lithium aluminum hydride (2.66 g, 70 mmol) in THF (120 mL) at 0° C. was added 2-ethoxycarbonylmethyl-6-methoxy-nicotinic acid ethyl ester (5.26 g, 22 mmol). The reaction mixture was stirred at room temperature for 10 minutes and then at reflux for 80 minutes. The reaction mixture was cooled to 0° C. and to it was successively added water (3 mL), aqueous sodium hydroxide (15%, 3 mL) and water (9 mL). The resulting mixture was filtered and concentrated to give the product (3.68 g, 91.3%). ¹H NMR (300 MHz, CDCl₃): δ (ppm) 7.57 (d, 1H), 6.63 (d, 1H), 4.63 (s, 2H), 4.07 (t, 2H), 3.92 (s, 3H), 3.02 (t, 2H). Using the above general procedure, the following compounds were synthesized:

| Ex- | | | |
|---|---|---|---|
| ample | Structure | Name | Yield |
| Ex- ample 4.2 | 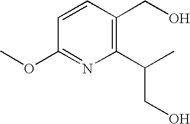 | 2-(3- Hydroxymethyl- 6- methoxy-pyridin- 2-yl)-propan-1-ol | 4.38 g, 95% |

| | |
|---|---|
| NMR | ¹H NMR (300 MHz, CDCl₃): δ(ppm) 7.71 (d, 1 H), 6.57 |
| | (d, 1 H), 4.72 (d, 1 H), 4.45 (d, 1 H), 3.91 (s, 3 H), 3.80 |
| | (m, 2 H), 3.30 (m, 1 H), 1.28 (d, 3 H). |

### Example 4.3

### 2-[3-(hydroxymethyl)-6-(trifluoromethyl)pyridin-2-yl]ethanol

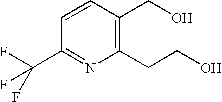

To a solution of 2.5 M n-butyl lithium (11.7 mL, 29.25 mmol) in tetrahydrofuran (100 ml), diisopropylamine (1.479 g, 14.62 mmol) was added at −78° C. After the mixture was placed in ice bath for 5 min, cooled to −78° C. again. A solution of 2-methyl-6-(trifluoromethyl)nicotinic acid (3.0 g, 14.62 mmol) in tetrahydrofuran (50 mL) was added to the reaction mixture via syringe and reaction mixture was stirred at −78° C. for an hour. Then a dry carbon dioxide gas was bubbled to the reaction mixture for 30 min. the reaction mixture was concentrated to dry and the residue was redissolved in tetrahyrofuran (100 mL). A solution of AlH₃ in tetrahydrofuran which was prepared from aluminum chloride (2.933 g, 22 mmol) in tetrahydrofuran (22 mL) reacted with 1M lithium aluminum hydride (66 ml, 666 mmol) in tetrahydrofuran was added to the reaction mixture slowly at 0° C. and the reaction mixture was stirred at room temperature overnight. The reaction mixture was diluted with diethyl ether and quenched with 5M sodium hydroxide carefully at 0° C. the mixture was dried with magnesium sulfate, filtered. The product was purified by column chromatography to give the title compound 400 mg (12.4%). ¹H NMR (300 MHz, CDCl₃): δ(ppm) 7.88 (d, 1H), 7.54 (d, 1H), 4.33 (s, 2H), 4.14 (br, 2H), 3.93 (t, 2H), 3.02 (t, 2H).

### Example 5.1

### 2-(2-Chloro-ethyl)-3-chloromethyl-6-methoxy-pyridine

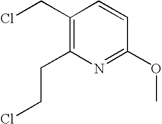

A mixture of 2-(3-hydroxymethyl-6-methoxy-pyridin-2-yl)-ethanol (3.68 g, 20 mmol), thionyl chloride (16 mL) and chloroform (80 mL) was stirred at room temperature overnight and then heated at reflux for 1 hour. The reaction mixture was concentrated, diluted with ethyl acetate and washed with aqueous sodium carbonate containing ice. The organic layer was dried over sodium sulfate and concentrated. The residue was purified on silica gel using hexanes:ethyl acetate (10:0 to 9.5:0.5) to give the product (3.23 g, 73%). ¹H NMR (300 MHz, CDCl₃): δ (ppm) 7.52 (d, 1H), 6.62 (d, 1H), 4.61 (s, 2H), 4.03 (t, 2H), 3.93 (s, 3H), 3.28 (t, 2H).

### Example 6.1

### [2-(2-Chloro-ethyl)-6-methoxy-pyridin-3-yl]-acetonitrile

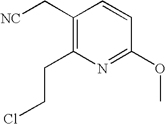

To a suspension of sodium cyanide (808 mg, 16.5 mmol) in N,N-dimethylformamide (20 mL) was added 2-(2-chloro-ethyl)-3-chloromethyl-6-methoxy-pyridine (2.88 g, 15 mmol). The reaction mixture was stirred at 25° C. for 3 hours. To the reaction mixture was added water and ethyl acetate. The organic layer was separated and washed further with water. The organic layer was dried over sodium sulfate and concentrated. The residue was purified on silica gel using hexanes:ethyl acetate (10:0 to 8:2) to give the product (2.4 g, 88%). ¹H NMR (300 MHz, CDCl₃): δ (ppm) 7.56 (d, 1H), 6.67 (d, 1H), 4.04 (t, 2H), 3.93 (s, 3H), 3.71 (s, 2H), 3.16 (t, 2H).

### Example 7.1

### [2-(2-Iodo-ethyl)-6-methoxy-pyridin-3-yl]-acetonitrile

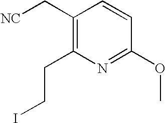

To the crude mixture of [2-(2-chloro-ethyl)-6-methoxy-pyridin-3-yl]-acetonitrile (875 mg, 4.8 mmol), sodium iodide (5.0 g, 33.3 mmol) and acetone (10 mL) was heated at 58° C. for 2 days. The reaction mixture was concentrated, diluted with dichloromethane, filtered and concentrated once again to give the product (1.30 g). ¹H NMR (300 MHz, CDCl₃): δ (ppm) 7.55 (d, 1H), 6.66 (d, 1H), 3.93 (s, 3H), 3.68 (s, 2H), 3.63 (t, 2H), 3.3 (t, 2H).

### Example 8.1

### [2-(2-Benzylamino-ethyl)-6-methoxy-pyridin-3-yl]-acetonitrile

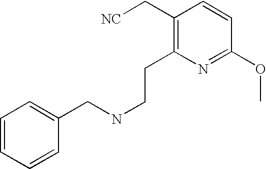

A solution of [2-(2-iodo-ethyl)-6-methoxy-pyridin-3-yl]-acetonitrile (7.9 g, 29 mmol) in butanol (40 mL) was added to a mixture of benzylamine (11 mL, 0.1 mol) in butanol (80 mL) at 90° C. The reaction mixture was stirred at 100° C. for 1 hour. The reaction mixture was concentrated, diluted with ethyl acetate and washed with aqueous sodium carbonate. The organic layer was dried over sodium sulfate, concentrated and purified on silica gel using dichloromethane:2M NH₃ in methanol (10:0 to 9.5:0.5) to give the product (5.2 g, 90% pure). ¹H NMR (300 MHz, CDCl₃): δ (ppm) 7.54 (d, 1H), 6.62 (d, 1H), 3.86 (m, 5H), 3.66 (s, 2H), 3.13 (t, 2H), 2.91 (t, 2H).

### Example 9.1

### [2-(2-Benzylamino-ethyl)-6-methoxy-pyridin-3-yl]-acetic acid methyl ester

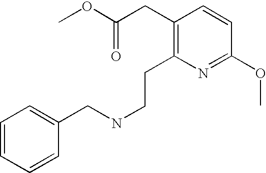

A mixture of [2-(2-benzylamino-ethyl)-6-methoxy-pyridin-3-yl]-acetonitrile (5.2 g) and a solution of hydrochloric acid in methanol (12%, 150 mL) was heated at reflux overnight. The reaction mixture was cooled to room temperature and sodium bicarbonate was then added. After stirring for 30 minutes, methanol was evaporated. To the residue was added ethyl acetate and water. The aqueous layer was separated and further extracted with ethyl acetate. The combined organic layer was dried over sodium sulfate and concentrated to give the product (4.85 g). ¹H NMR (300 MHz, CDCl₃): δ (ppm) 7.27 (m, 6H), 6.56 (d, 1H), 3.86 (m, 5H), 3.67 (m, 3H), 3.59 (s, 2H), 3.09 (t, 2H), 2.94 (t, 2H).

### Example 10.1

### 7-Benzyl-2-methoxy-5,7,8,9-tetrahydro-pyrido[2,3-d]azepin-6-one

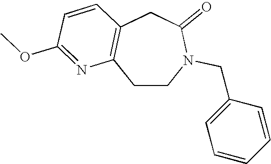

A mixture of [2-(2-benzylamino-ethyl)-6-methoxy-pyridin-3-yl]-acetic acid methyl ester (1.6 g, 5.4 mmol) in N,N-dimethylformamide (18 mL) was heated at 200° C. in a microwave for 4 hours. The above reaction was repeated using 3.2 g of substrate. The two reaction mixtures were combined, diluted with ethyl acetate and washed with water. The organic layer was dried over sodium sulfate, concentrated and purified on silica gel using hexanes:ethyl acetate (9:1 to 1:1) to give the product (2.0 g, 47%). ¹H NMR (300 MHz, CDCl₃): δ (ppm) 7.32 (m, 6H), 6.54 (d, 1H), 4.67 (s, 2H), 3.86 (s, 5H), 3.7 (t, 2H), 2.99 (t, 2H).

### Example 11.1

### 7-Benzyl-2-methoxy-6,7,8,9-tetrahydro-5H-pyrido[2,3-d]azepine

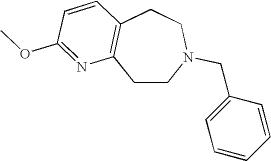

To a suspension of lithium aluminum hydride (1.9 g, 50 mmol) in tetrahydrofuran (60 mL) at −78° C., was added to aluminum chloride (2.4 g, 18 mmol) followed by a solution of 7-benzyl-2-methoxy-5,7,8,9-tetrahydro-pyrido[2,3-d]azepin-6-one (1.6 g, 6 mmol) in tetrahydrofuran (40 mL). The reaction mixture was stirred at room temperature for 2 days. To the reaction mixture at 0° C. was slowly and successively added water (2 mL), aqueous sodium hydroxide (1N, 2 mL) and water (4 mL). The resulting mixture was filtered through Celite® and concentrated. The above reaction was repeated using 422 mg of substrate. The combined residue was purified on silica gel using dichloromethane:2M NH₃ in methanol (10:0 to 9.5:0.5) to give the product (1.8 g, 95%). The product was treated with hydrochloric acid in diethyl ether to give the hydrochloric acid salt (2 salt equivalents). ¹H NMR (300 MHz, CDCl₃): δ (ppm) 7.31 (m, 6H), 6.48 (d, 1H), 3.9 (s, 3H), 3.65 (s, 2H), 3.07 (m, 2H), 2.82 (m, 2H), 2.68 (m, 2H), 2.61 (m, 2H).

### Example 12.1

### 2-[3-(Chloromethyl)-6-methoxypyridin-2-yl]ethyl methanesulfonate

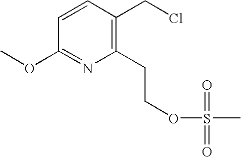

The title compound from Example 3 (24.70 g, 134.8 mmol) was dissolved in dichloromethane (500 mL) under a nitrogen atmosphere and cooled to −30° C. Then triethylamine (30.01 g, 296.6 mmol) and methyl sulfonyl chloride (33.98 g, 296.6 mmol) were added. The reaction was allowed to warm to r.t. and was stirred overnight. The reaction mixture was diluted with hexanes (400 mL) and filtered to remove solids. The filtrate was concentrated and redissolved in ethyl acetate, washing with saturated sodium bicarbonate solution and brine. The organic layer was dried over magnesium sulfate, filtered and concentrated to give the title compound as pale yellow oil (36.38 g, 96%). ¹H NMR (300 MHz, CDCl₃): δ(ppm) 7.51 (d, 1H), 6.60 (d, 1H), 4.75 (t, 2H), 4.57 (s, 2H), 3.90 (s, 3H), 3.25 (t, 2H), 2.92 (s, 3H).

Using the above general procedure, the following compounds were synthesized:

| Ex- | | | |
|---|---|---|---|
| ample | Structure | Name | Yield |
| Ex- ample 12.2 | 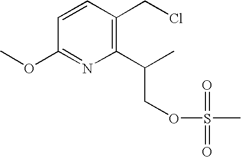 | 2-[3- (chloromethyl)- 6-methoxy- pyridin- 2yl]propyl methane- sulfonate | 6.18 g, 95% |
| NMR | ¹H NMR (300 MHz, CDCl₃): δ(ppm) 7.53 (d, 1 H), 6.56 | | |
| | (d, 1 H), 4.66 (m, 3 H), 4.43 (m, 1 H), 3.94 (s, 3 H), | | |
| | 3.62 (m, 1 H), 2.88 (s, 3 H), 1.31 (q, 3 H). | | |
| Ex- ample 12.3 | 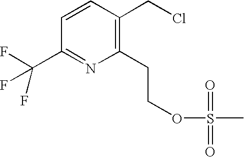 | 2-[3- (chloromethyl)- 6- (trifluoro- methyl) pyridin-2-yl] ethyl methane- sulfonate | 300 mg, 100% |

| | |
|---|---|
| NMR | ¹H NMR (300 MHz, CDCl₃): δ(ppm) 7.93 (d, 1 H), 7.55 |
| | (d, 1 H), 4.74 (t, 2 H), 4.63 (s, 2 H), 3.35 (t, 2 H), 2.94 (s, 3 H). |

### Example 13.1

### 2-[3-(Cyanomethyl)-6-methoxypyridin-2-yl]ethyl methanesulfonate

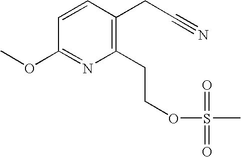

The title compound from Example 4 (36.38 g, 130.05 mmol) was dissolved in dimethylformamide (400 mL) and cooled to 0° C. Sodium cyanide (6.69 g, 135.55 mmol) was added and the reaction mixture was allowed to warm up to r.t. overnight. The reaction mixture was filtered through celite filtering agent. The filtrate was washed with 25% saturated sodium bicarbonate solution and extracted with portions of ethyl acetate. The organic extracts were dried over sodium sulphate, filtered and concentrated. The product was purified by column chromatography (30-50% ethyl acetate in hexanes) to give the title compound (21.13 g, 60%). ¹H NMR (300 MHz, CDCl₃): δ(ppm) 7.48 (d, 1H), 6.57 (d, 1H), 4.65 (t, 2H), 3.84 (s, 3H), 3.62 (s, 2H), 3.07 (t, 2H), 2.90 (s, 3H).

Using the above general procedure, the following compounds were synthesized:

| Ex- | | | |
|---|---|---|---|
| ample | Structure | Name | Yield |
| Ex- ample 13.2 | 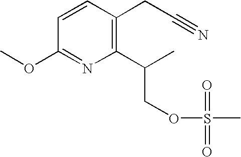 | 2-[3- (cyanomethyl)- 6- methoxy- pyridin- 2-yl]propyl methane- sulfonate | 2.81 g, 47% |
| NMR | ¹H NMR (300 MHz, CDCl₃): δ(ppm) 7.55 (d, 1 H), 6.66 | | |
| | (d, 1 H), 4.61 (t, 3 H), 4.41 (q, 1 H), 3.71 (d, 1 H), 3.41 | | |
| | (m, 1 H), 2.90 (s, 3 H), 1.32 (d, 3 H). | | |
| Ex- ample 13.3 | 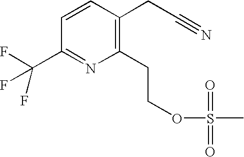 | 2-[3- (cyanomethyl)- 6- (trifluoro- methyl) pyridin-2-yl] ethyl methane- sulfonate | 41.6 mg, 14% |

| | |
|---|---|
| NMR | ¹H NMR (300 MHz, CDCl₃): δ(ppm) 7.98 (d, 1 H), 7.66 |
| | (d, 1 H), 4.80 (t, 2 H), 3.88 (s, 2 H), 3.29 (t, 2 H), 3.00 |
| | (s, 3 H). |

### Example 14.1

### 6,7,8,9-Tetrahydro-5H-pyrido[2,3-d]azepin-2-ol

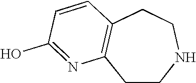

A mixture of 7-benzyl-2-methoxy-6,7,8,9-tetrahydro-5H-pyrido[2,3-d]azepine (35 mg, 0.14 mmol) (the compound from Example 11.1), hydrobromic acid in ethanol (1.3 mL) and acetic acid (1.3 mL) was heated at 80° C. overnight. The reaction mixture was concentrated, diluted with ethyl acetate and washed with aqueous sodium carbonate. The organic layer was dried over sodium sulfate and concentrated to give the crude amide. The amide was hydrogenated in methanol using palladium (10% on carbon). The reaction mixture was filtered through Celite®, concentrated and purified on silica gel using dichloromethane:2M NH₃ in methanol (10:0 to 9.2:0.8) to give the product (2 mg). The product was treated with hydrochloric acid in diethyl ether to give the hydrochloric acid salt (2 salt equivalents).

### Example 15.1

### 2-Methoxy-6,7,8,9-tetrahydro-5H-pyrido[2,3-d]azepine

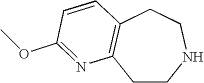

### Method A

A mixture of 7-benzyl-2-methoxy-6,7,8,9-tetrahydro-5H-pyrido[2,3-d]azepine (1.05 g, 4.2 mmol), (the compound from Example 11.1), palladium hydroxide (20% on carbon, 180 mg) and methanol (50 mL) was stirred under hydrogen (36 psi) for 6 hours. The reaction mixture was filtered through Celite® and concentrated to give the product (565 mg). The product was treated with hydrochloric acid in diethyl ether to give the hydrochloric acid salt (2 salt equivalents). ¹H NMR (300 MHz, CDCl₃): δ (ppm) 7.29 (d, 1H), 6.48 (d, 1H), 3.91 (s, 3H), 3.08 (m, 2H), 2.98 (m, 4H), 2.82 (m, 2H).

### Method B

To a mixture of aluminum chloride (1.8 g, 13.3 mmol) and lithium aluminum hydride (1.0 g, 26.6 mmol) at −78° C., dry diethyl ether (30 mL) was added carefully. After being stirred at r.t. for 30 min, the reaction mixture was cooled down to −78° C. again. Then a solution of 2-[3-(cyanomethyl)-6-methoxypyridin-2-yl]ethyl methanesulfonate (3.6 g, 13.3 mmol) (the compound from Example 13.1) in tetrahydrofuran (30 mL) was added slowly. The reaction mixture was stirred at 0° C. for an hour and a half. Water (200 mL) and saturated sodium carbonate (10 mL) were used to quench the reaction. The reaction mixture was extracted with ethyl acetate, dried with sodium sulfate, concentrated by Rotavapor. The residue was mixed with diisopropylethylamine (3.36 g, 26 mmol) in acetonitrile (45 ml) and stirred at 30° C. for 24 hours, concentrated again, saturated sodium carbonate (15 mL) was added. The mixture was extracted with ethyl acetate, dried, purified by chromatography to give the title compound (1.25 g, 52.7%).

Using the method B general procedure, the following compounds were synthesized:

| Ex- | | | |
|---|---|---|---|
| ample | Structure | Name | Yield |
| Ex- ample 15.2 | 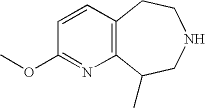 | 2-Methoxy-9- methyl-6,7,8,9- tetrahydro-5H- pyrido[2,3- d]azepine | 0.93 g, 49% |
| NMR | ¹H NMR (300 MHz, CDCl₃): δ(ppm) 7.34 (m, 1 H), 6.55 | | |
| | (d, 1 H), 3.92 (s, 3 H), 3.19 (m, 1 H), 3.06 (m, 2 H), 2.77 | | |
| | (m, 4 H), 1.96 (br s, 2 H), 1.34 (d, 3 H). | | |
| Ex- ample 15.3 | 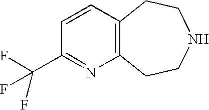 | 2- (trifluoromethyl)- 6,7,8,9- tetrahydro- 5H-pyrido[2,3- d]azepine | 4.7 mg, 34% |

| | |
|---|---|
| NMR | ¹H NMR (300 MHz, CDCl₃): δ(ppm) 7.53 (d, 1 H), 7.44 |
| | (d, 1 H), 3.26 (m, 2 H), 3.04 (m, 6 H), 2.54 (br, 1 H). |

### Example 16.1

### tert-Butyl 2-methoxy-5,6,8,9-tetrahydro-7H-pyrido[2,3-d]azepine-7-carboxylate

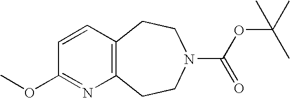

To a solution of the title compound Example 13.1 (1.07 g, 6 mmol) and diisopropylethylamine (1.5 mL) in dichlormethane (30 mL) at −50° C., di-tert-butyl dicarbonate (1.6 g, 7.2 mmol) was added. The reaction mixture was stirred at room temperature for two hours, then quenched with water and extracted with dichloromethane. The product was purified by column chromatograph with 10% ethyl acetate in hexanes to give the title compound 1.188 g (71%).

### Example 16.2

### tert-butyl 3-bromo-2-methoxy-5,6,8,9-tetrahydro-7H-pyrido[2,3-d]azepine-7-carboxylate

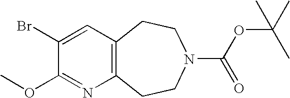

tert-Butyl 2-methoxy-5,6,8,9-tetrahydro-7H-pyrido[2,3-d]azepine-7-carboxylate (140 mg, 0.5 mmol) was mixed with sodium acetate (50 mh, 0.6 mmol) in dichloromethane at 0° C. Then Bromine (96 mg, 0.6 mmol) was added. the reaction mixture was filtered and concentrated to dry to give the title compound 200 mg (112%) which can be carried on for the next step reaction without further purification. ¹H NMR (300 MHz, CDCl₃): δ(ppm) 7.55 (s, 1H), 3.99 (s, 3H), 3.58 (m, 4H), 3.04 (m, 2H), 2.78 (m, 2H), 1.49 (s, 9H).

### Example 17.1

### 6,7,8,9-Tetrahydro-5H-pyrido[2,3-d]azepin-2-ol dihydrobromide

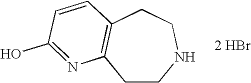

A mixture of 2-(6-methoxy-3-methyl-pyridin-2-yl)-ethyl]-methyl-amine (549 mg, 3.3 mmol), hydrobromic acid in ethanol (16 mL) and acetic acid (16 mL) was heated at 88° C. for 1 day. The reaction mixture was concentrated and the residue was triturated with hexanes to give the product (879 mg). Using the method B general procedure, the following compounds were synthesized:

| Example 17.2 | 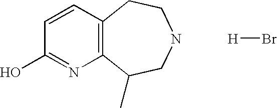 | 9-Methyl-6,7,8,9- tetrahydro-5H- pyrido [2,3-d]azepin-2- ol; hydrobromide | Not taken |
|---|---|---|---|
| NMR | ¹H NMR (300 MHz, CDCl₃): δ(ppm) not characterized | | |
| Example 17.3 | 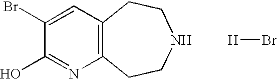 | 3-bromo-6,7,8,9- tetrahydro-5H- pyrido[2,3- d]azepin-2-ol; hydrobromide | Not taken |

| | |
|---|---|
| NMR | ¹H NMR (300 MHz, CDCl₃): δ(ppm) not characterized |

### Example 18.1

### 2-Chloro-6,7,8,9-tetrahydro-5H-pyrido[2,3-d]azepine

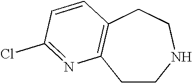

A mixture of 6,7,8,9-tetrahydro-5H-pyrido[2,3-d]azepin-2-ol dihydrobromide (114 mg, 0.38 mmol) and phosphorus oxychloride (1.5 mL) was heated at 120° C. for 1 hour. The reaction mixture was concentrated and to the residue was added aqueous sodium carbonate. The resulting mixture was extracted with ethyl acetate and the organic layer concentrated. The residue was purified on silica gel using dichloromethane:2M NH₃ in methanol (10:0 to 9.5:0.5) to give the product (3 mg). The product was treated with hydrochloric acid in diethyl ether to give the hydrochloric acid salt (2 salt equivalents). ¹H NMR (300 MHz, CDCl₃): δ (ppm) 7.36 (d, 1H), 7.08 (d, 1H), 3.15 (m, 2H), 3.01 (m, 3H), 2.89 (m, 2H), 2.62 (m, 1H).

### Example 19.1

### tert-Butyl 2-hydroxy-5,6,8,9-tetrahydro-7H-pyrido[2,3-d]azepine-7-carboxylate

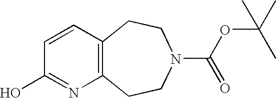

The title compound from Example 14.1 (5.72 g, 17.54 mmol) was suspended in dichloromethane (90 mL) under a nitrogen atmosphere and cooled to 0° C. Diisopropylethyamine (7.93 g, 61.39 mmol) was added to the suspension with stirring. In a separate flask, di-tert-butyl dicarbonate (8.04 g, 36.83 mmol) was dissolved in dichloromethane (50 mL) under a nitrogen atmosphere. This solution was added slowly to the main reaction vessel via cannula. The reaction was stirred at r.t. for 2 hours. The reaction mixture was washed with a 50% saturated solution of ammonium chloride and the aqueous phase was extracted with portions of dichloromethane. The combined organic extracts were washed with brine, dried over manesium sulfate, filtered and concentrated to give the title compound as a place brown solid (4.64 g, quant.). ¹H NMR (300 MHz, CDCl₃): δ(ppm) 13.53 (br s, 1H), 7.25 (d, 1H), 6.38 (d, 1H), 3.55 (m, 4H), 2.94 (m, 2H), 2.68 (m, 2H), 1.46 (s, 9H).

Using the above general procedure, the following compounds were synthesized:

| Example | Structure | Name | Yield |
|---|---|---|---|
| Example 19.2 | 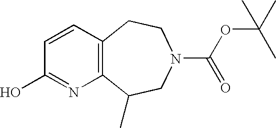 | tert-butyl 2- hydroxy-9-methyl- 5,6,8,9-tetrahydro- 7H-pyrido[2,3- d]azepine-7- carboxylate | Not taken |
| NMR | ¹H NMR (300 MHz, CDCl₃): δ(ppm) not characterized | | |
| Example 19.3 | 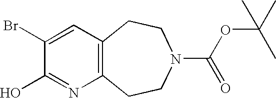 | tert-butyl 3-bromo- 2-hydroxy-5,6,8,9- tetrahydro-7H- pyrido[2,3- d[azepine-7- carboxylate | Not taken |

| | |
|---|---|
| NMR | ¹H NMR (300 MHz, CDCl₃): δ(ppm) not characterized |

### Example 20.1

### tert-Butyl 2-{[(trifluoromethyl)sulfonyl]oxy}-5,6,8,9-tetrahydro-7H-pyrido[2,3-d]azepine-7-carboxylate

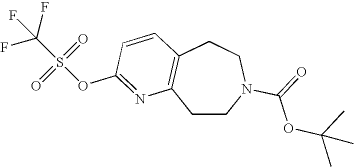

The title compound from Example 16.1 (4.64 g, 17.54 mmol) was dissolved in dichloromethane (150 mL) and cooled to 0° C. under nitrogen. To this solution was added diisopropylethylamine (2.83 g, 21.93 mmol) and triflic anhydride (6.19 g, 21.93 g) and the reaction was allowed to warm to r.t. over 2 hours. The reaction mixture was concentrated and partitioned between ethyl acetate and water. The organic phase was washed with 50% sat. ammonium chloride and brine. The organic phase was neutralized with sat. sodium bicarbonate and concentrated to give the title compound (1.94 g, 28%). ¹H NMR (300 MHz, CDCl₃): δ(ppm) 7.59 (d, 1H), 6.94 (d, 1H), 3.61 (br d, 4H), 3.12 (t, 2H), 2.93 (t, 2H), 1.49 (s, 9H).

Using the above general procedure, the following compounds were synthesized:

| Example | Structure | Name | Yield |
|---|---|---|---|
| Example 20.2 | 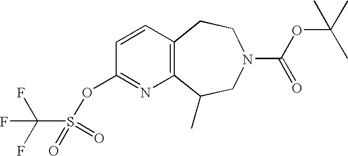 | tert-butyl 9-methyl- 2- {[(trifluoromethyl) sulfonyl]oxy}-5,6,8,9- tetrahydro-7H- pyrido[2,3- d]azepine-7- carboxylate | 1.11 g, 56% |

| NMR | ¹H NMR (300 MHz, CDCl₃): δ(ppm) 7.58 (d, 1 H), 6.93 (d, 1 H), | | |
|---|---|---|---|
| | 3.64 (m, 3 H), 3.27 (m, 2 H), 2.91 (m, 2 H), 1.47 (s, 9 H), 1.36 (d, | | |
| | 3 H). | | |
| Example 20.3 | 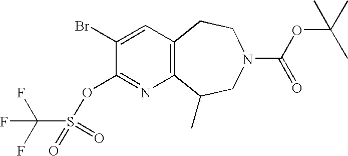 | tert-butyl 3-bromo- 9-methyl-2- {[(trifluoromethyl) sulfonyl]oxy}-5,6,8,9- tetrahydro-7H- pyrido[2,3- d]azepine-7- carboxylate | 1.11 g, 56% |

| | |
|---|---|
| NMR | ¹H NMR (300 MHz, CDCl₃): δ(ppm) 7.78 (s, 1 H), 3.61 (m, 3 H), |
| | 3.27 (m, 4 H), 3.08 (m, 2 H), 2.91 (m, 2 H), 1.50 (s, 9 H). |

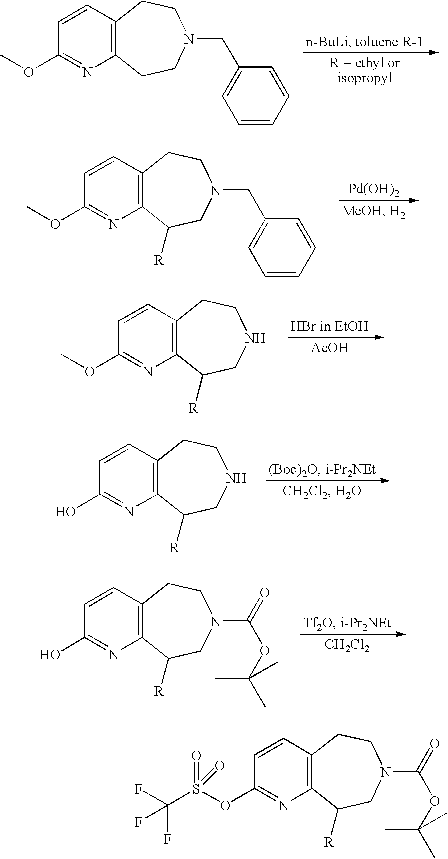

### Example 20.4

### tert-Butyl 9-ethyl-2-{[(trifluoromethyl)sulfonyl]oxy}-5,6,8,9-tetrahydro-7H-pyrido[2,3-d]azepine-7-carboxylate

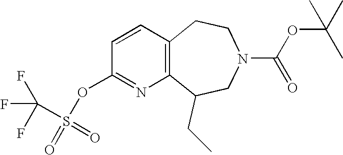

To a solution of 7-benzyl-2-methoxy-6,7,8,9-tetrahydro-5H-pyrido[2,3-d]azepine (500 mg, 1.86 mmol) in toluene (8 mL) at −58° C., n-BuLi (1.5 mL of 2.5 M in hexanes, 3.75 mmol) was added. After the mixture was stirred at 0° C. for two hours, iodoethane (650 μL, 8 mmol) was added and the reaction mixture was stirred at room temperature overnight. The reaction mixture was quenched with saturated ammonium chloride and extracted with ethyl acetate, dried and concentrated by Rotavapor. The residue was mixed with 20% Pd(OH)₂ (400 mg) and methanol (30 mL) and stirred under H₂ for a week. The reaction mixture was filtered and the filtrate was concentrated again to give 9-ethyl-2-methoxy-6,7,8,9-tetrahydro-5H-pyrido[2,3-d]azepine. This compound was mixed with 20% wt HBr in ethanol (16 mL) and acetic acid (16 mL) and heated at 90° C. overnight and then concentrated to give 9-ethyl-6,7,8,9-tetrahydro-5H-pyrido[2,3-d]azepin-2-ol dihydrobromide salt. The salt was mixed with diisopropylethyamine (2.5 mL) and di-tert-butyl dicarbonate (570 mg, 2.5 mmol) in dichloromethane (20 mL) and water (10 mL) at 0° C. and stirred for two hours. The organic layer was separated and dried, concentrated to give tert-butyl 9-ethyl-2-hydroxy-5,6,8,9-tetrahydro-7H-pyrido[2,3-d]azepine-7-carboxylate. This intermediate was mixed with diisopropylethylamine (1 mL) and triflic anhydride (420 μL, 2.5 mmol)) in dichloromethane at −50° C. and stirred overnight. After work-up, purified by column chromatography with 20% ethyl acetate in hexanes to give the title compound 284 mg (37% in total). ¹H NMR (300 MHz, CDCl₃): δ(ppm) 7.58 (d, 1H), 6.93 (d, 1H), 2.6-3.9 (m, 7H), 1.95 (m, 2H), 1.49 (s, 9H), 1.37 (m, 3H).

Using the above general procedure, the following compounds were synthesized:

| Example | Structure | Name | Yield |
|---|---|---|---|
| Example 20.5 | 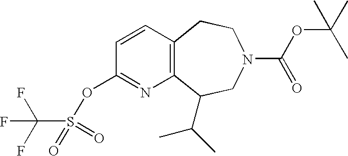 | tert-butyl 9- isopropyl-2- {[(trifluoromethyl) sulfonyl]oxy}-5,6,8,9- tetrahydro-7H- pyrido[2,3- d]azepine-7- carboxylate | 574 mg, 45% |

| | |
|---|---|
| NMR | ¹H NMR (300 MHz, CDCl₃): δ(ppm) 7.56 (d, 1 H), 6.93 (d, 1 H), |
| | 2.80-3.90 (m, 7 H), 2.30 (m, 1 H), 1.45 (s, 9 H), 1.08 (d, 3 H), |
| | 0.81 (d, 3 H). |

### Example 21.1

### tert-Butyl ester 2-cyano-5,6,8,9-tetrahydro-pyrido[2,3-d]azepine-7-carboxylate

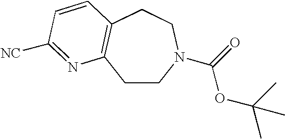

A mixture of tert-Butyl 2-{[(trifluoromethyl)sulfonyl]oxy}-5,6,8,9-tetrahydro-7H-pyrido[2,3-d]azepine-7-carboxylate (50 mg, 0.13 mmol), zinc cyanide (50 mg, 0.4 mmol), tetrakis(triphenylphosphine)palladium (25 mg) and N,N-dimethylformamide (0.6 mL) was stirred at 100° C. for 30 minutes. The reaction mixture was cooled to room temperature and diluted with water and ethyl acetate. The organic layer was separated, washed with water (3×1 mL), dried over sodium sulfate and concentrated. The residue was purified on silica gel using hexanes:ethyl acetate (10:0 to 7:3) to give the product (12 mg, 33.8%). ¹H NMR (300 MHz, CDCl₃): δ(ppm) 7.55 (d, 1H), 7.49 (d, 1H), 3.62 (m, 4H), 3.21 (m, 2H), 2.96 (m, 2H), 1.50 (s, 9H).

### Example 21.2

### tert-Butyl 2-isopropenyl-5,6,8,9-tetrahydro-pyrido[2,3-d]azepine-7-carboxylate

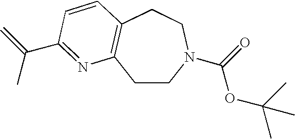

To a solution of 2-bromopropene (266 mL, 3.0 mmol) in diethyl ether (3 mL) at −78° C., was added tert-butyllithium (1.7M in pentane, 3.5 mL, 6.0 mmol). The reaction mixture was stirred at −78° C. for 1 hour and then a solution of zinc bromide (810 mg, 3.6 mmol) in tetrahydrofuran (3.6 mL) was added. The reaction mixture was warmed to room temperature and stirred at room temperature for 30 minutes. A separate mixture of tert-butyl 2-trifluoromethanesulfonyloxy-5,6,8,9-tetrahydro-pyrido[2,3-d]azepine-7-carboxylate (100 mg, 0.26 mmol), bis(dibenzylideneacetone)palladium (5 mg, 0.01 mmol), tri-2-furylphosphine (4.5 mg, 0.02 mmol) and tetrahydrofuran (0.6 mL) was stirred at room temperature for 15 minutes. To this reaction mixture was added the zinc reagent described above (2 equivalents taken from the above reaction mixture) at room temperature. The reaction mixture was heated at 50° C. for 45 minutes. After work-up, the residue was purified on silica gel using hexanes:ethyl acetate (10:0 to 8.5:1.5) to give the product (45 mg, 64%). ¹H NMR (300 MHz, CDCl₃): δ(ppm) 7.37 (d, 1H), 7.21 (d, 1H), 5.89 (s, 1H), 5.25 (s, 1H), 3.60 (m, 4H), 3.17 (m, 2H), 2.87 (m, 2H), 2.19 (s, 3H), 1.50 (s, 9H).

Using the above general procedure, the following compounds were synthesized:

| Example | Structure | Name | Yield |
|---|---|---|---|
| Example 21.3 | 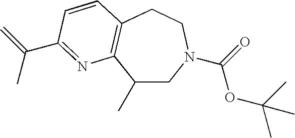 | tert-butyl 2- isopropenyl-9- methyl-5,6,8,9- tetrahydro-7H- pyrido[2,3- d]azepine-7- carboxylate | 63 mg, 51% |

| | |
|---|---|
| NMR | ¹H NMR (300 MHz, CDCl₃): δ(ppm) 7.35 (d, 1 H), 7.21 (d, 1 H), |
| | 5.91 (s, 1 H), 5.25 (s, 1 H), 3.51 (m, 5 H), 2.87 (m, 2 H), 2.20 (s, |
| | 3 H), 1.49 (s, 9 H), 1.37 (d, 3 H). |

### Example 21.4

### tert-Butyl 2-phenyl-5,6,8,9-tetrahydro-pyrido[2,3-d]azepine-7-carboxylate

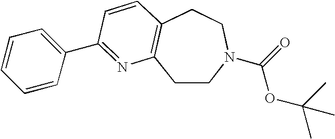

A mixture of 2-trifluoromethanesulfonyloxy-5,6,8,9-tetrahydro-pyrido[2,3-d]azepine-7-carboxylic acid tert-butyl ester (100 mg, 0.26 mmol), aqueous sodium carbonate (2M, 0.5 mL), tetrakis(triphenylphosphine)palladium (15 mg), phenylboronic acid (80 mg, 0.5 mmol) and N,N-dimethylformamide (1.5 mL) was heated at 88° C. under nitrogen for 2 hours. The reaction mixture was extracted with ethyl acetate and the organic layer was concentrated. The residue was purified on silica gel using hexanes:ethyl acetate (10:0 to 7:3) to give the product. ¹H NMR (300 MHz, CDCl₃): δ(ppm) 7.98 (d, 2H), 7.40 (m, 5H), 3.64 (m, 4H), 3.26 (m, 2H), 2.92 (m, 2H), 1.52 (s, 9H).

Using the above general procedure, the following compounds were synthesized:

| Example | Structure | Name | Yield |
|---|---|---|---|
| Example 21.5 | 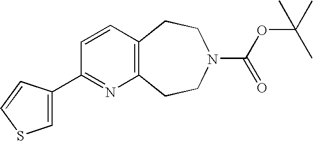 | tert-butyl 2-(3- thienyl)-5,6,8,9- tetrahydro-7H- pyrido[2,3- d]azepine-7- carboxylate | 83 mg, 100% |
| NMR | ¹H NMR (300 MHz, CDCl₃): δ(ppm) 7.87 (s, 1 H), 7.63 (d, 1 H), | | |
| | 7.40 (m, 3 H), 3.60 (m, 4 H), 3.21 (m, 2 H), 2.89 (m, 2 H), 1.51 (s, | | |
| | 9 H). | | |
| Example 21.6 | 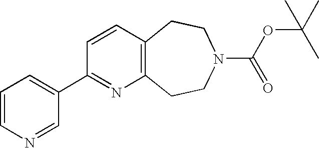 | tert-butyl 2-pyridin- 3-yl-5,6,8,9- tetrahydro-7H- pyrido[2,3- d]azepine-7- carboxylate | 100 mg, 100% |
| NMR | ¹H NMR (300 MHz, CDCl₃): δ(ppm) 9.17 (s, 1 H), 8.62 (m, 1 H), | | |
| | 8.32 (m, 1 H), 7.52 (s, 2 H), 7.38 (m, 1 H), 3.64 (m, 4 H), 3.25 (m, | | |
| | 2 H), 2.94 (m, 2 H), 1.50 (s, 9 H) | | |
| Example 21.7 | 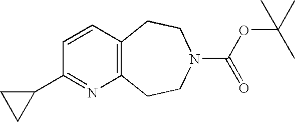 | tert-butyl 2- cyclopropyl-5,6,8,9- tetrahydro-7H- pyrido[2,3- d]azepine-7- carboxylate | 50 mg, 69% |
| NMR | ¹H NMR (300 MHz, CDCl₃): δ(ppm) 7.26 (d, 1 H), 6.80 (d, 1 H), | | |
| | 3.56 (m, 4 H), 3.09 (m, 2 H), 2.80 (m, 2 H), 2.04 (m, 1 H), 1.48 (s, | | |
| | 9 H), 0.92 (m, 4 H). | | |
| Example 21.8 | 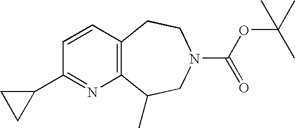 | tert-butyl 2- cyclopropyl-9- methyl-5,6,8,9- tetrahydro-7H- pyrido[2,3- d]azepine-7- carboxylate | 51 mg, 75% |

| | |
|---|---|
| NMR | ¹H NMR (300 MHz, CDCl₃): δ(ppm) 7.22 (d, 1 H), 6.84 (d, 1 H), |
| | 2.7-3.8 (m, 7 H), 1.99 (m, 1 H), 1.48 (s, 9 H), 1 .30 (d, 3 H), 0.93 |
| | (m, 4 H). |

### Example 21.9

### tert-Butyl 2-vinyl-5,6,8,9-tetrahydro-7H-pyrido[2,3-d]azepine-7-carboxylate

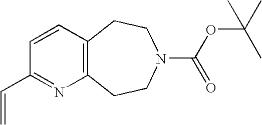

The title compound from Example 17.1 (0.454 g, 1.15 mmol), lithium chloride (0.146, 3.44 mmol) and tetrakis(triphenylphosphine)palladium (0.132 g, 0.115 mmol) were suspended in toluene (10 mL) under a nitrogen atmosphere. Tributyl vinyl tin (0.40 g, 1.26 mmol) was added to this suspension and the reaction was refluxed for 2 hours. The reaction mixture was concentrated onto silica gel and purified by column chromatography (20% EtOAc/Hexanes) to give the title compound (0.175 g, 56%). ¹H NMR (300 MHz, CDCl₃): δ(ppm) 7.29 (d, 1H), 7.05 (d, 1H), 6.71 (q, 1H), 6.08 (d, 1H), 5.35 (d, 1H), 3.52 (brt, 4H), 3.10 (br, 2H), 2.80 (br, 2H), 1.44 (s, 9H).

### Example 21.10

### tert-butyl 2-methyl-5,6,8,9-tetrahydro-7H-pyrido[2,3-d]azepine-7-carboxylate

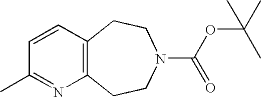

The title compound from Example 17.1 (100 mg, 0.26 mmol), Pd(dba)₂ (10 mg, 0.017 mmol) and PPh₃ (8 mg, 0.034 mmol) were mixed in tetrahydrofuran (2 mL) at room temperature under N₂ and stirred for 20 min. Me₂Zn (2M in THF, 0.3 mL, 0.6 mmol)) was added. The reaction mixture was heated to 50° C. for 2 hours. The reaction mixture was quenched with water and extracted with ethyl acetate and purified by column chromatography (EtOAc/Hexanes=1/2) to give the title compound (55 mg, 76%). ¹H NMR (300 MHz, CDCl₃): δ(ppm) 7.30 (d, 1H), 6.93 (d, 1H), 3.57 (m, 4H), 3.12 (m, 2H, 2.84 (m, 2H), 1.50 (s, 9H)

### Example 21.11

### 2-(Methyl-phenyl-amino)-5,6,8,9-tetrahydro-pyrido[2,3-d]azepine-7-carboxylic acid tert-butyl ester

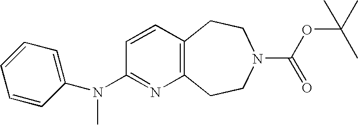

The title compound from Example 10.2 (0.200 g, 0.526 mmol), Pd2(dba)₃ (0.010 g, 0.0105 mmol), BINAP (0.0131 g, 0.0210 mmol), sodium tert-butoxide (0.071 g, 0.736 mmol) and methyl-phenyl-amine (0.068 g, 0.631 mmol) were stirred in toluene (5.0 mL) under a nitrogen atmosphere at 100° C. overnight. The reaction mixture was partitioned between ethyl acetate and water. The organic extracts were dried over magnesium sulfate, filtered, concentrated and purified by column chromatography to give the title compound (0.020 g, 11%). ¹H NMR (300 MHz, CDCl₃): δ(ppm) 7.37 (t, 2H), 7.26 (m, 2H), 7.16 (t, H), 7.06 (d, 1H), 6.37 (d, 1H), 3.59 (m, 4H), 3.49 (s, 3H), 3.06 (m, 2H), 2.75 (m, 2H), 1.50 (s, 9H).

Using the above general procedure, the following compounds were synthesized:

| | | | |
|---|---|---|---|
| Example 21.12 | 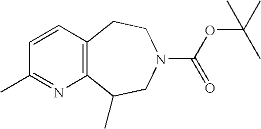 | tert-butyl 2,9- dimethyl-5,6,8,9- tetrahydro-7H- pyrido[2,3- d]azepine-7- carboxylate | 61 mg, 86% |

| | |
|---|---|
| NMR | ¹H NMR (300 MHz, CDCl₃): δ(ppm) 7.26 (d, 1 H), 6.90 (d, 1 H), |
| | 3.90 (m, 2 H), 3.33 (m, 4 H), 2.72 (m, 1 H), 2.49 (s, 3 H), 1.48 (s, 9 H), 1.31 |
| | (d, 3 H). |

### Example 21.13

### tert-Butyl 2-formyl-5,6,8,9-tetrahydro-7H-pyrido[2,3-d]azepine-7-carboxylate

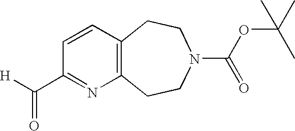

The title compound from Example 18.4 (0.175 g, 0.639 mmol) was dissolved in dichloromethane (20 mL) and cooled to −78° C. Ozone was piped into the solution until it turned blue. The reaction was stirred for a further 15 minutes at −78° C. after which oxygen was piped into the reaction for 5 minutes. The reaction mixture was removed from the ice bath and methylsulfonylmethane (0.169 g, 2.72 mmol) was added with stirring. After 15 minutes, the reaction was concentrated to give the title product with no further purification. ¹H NMR (300 MHz, CDCl₃): δ(ppm) 9.98 (s, 1H), 7.98 (d, 1H), 7.72 (d, 1H), 3.61 (t, 4H), 3.24 (m, 2H), 2.96 (m, 2H), 1.46 (s, 9H).

### Example 21.14

### tert-Butyl 2-[(dimethylamino)carbonyl]-5,6,8,9-tetrahydro-7H-pyrido[2,3-d]azepine-7-carboxylate

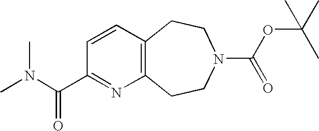

To a tetrahydrofuran (6.0 mL) solution of the title compound from Example 17.1 (0.120 g, 0.303 mmol) degassed with carbon monoxide, lithium bromide (0.0026 g, 0.030 mmol) and dimethyl amine (0.413 mL, 0.91 mmol) and Tetrakis palladium triphosphine (0.034 g, 0.030 mmol) in tetrahydrofuran (1 mL) were added. The reaction mixture was heated to 60° C. under a carbon monoxide atmosphere for 4 hours. The reaction was quenched and the product was purified by automated column chromatography to yield the title compound (0.040 g, 41%). ¹H NMR (300 MHz, CDCl₃): δ (ppm) 7.49 (d, 1H), 7.39 (d, 1H), 3.57 (br, 4H), 3.16 (br, 2H), 3.10 (d, 6H), 2.88 (br, 2H), 1.47 (s, 9H).

Using the above general procedure, the following compounds were synthesized:

| Example | Structure | Name | Yield |
|---|---|---|---|
| Example 21.15 | 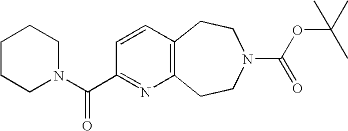 | tert-butyl 2- (piperidine-1- carbonyl)-5,6,8,9- tetrahydro- pyrido[2,3- d]azepine-7- carboxylate | 111 mg, 61% |
| NMR | ¹H NMR (300 MHz, CDCl₃): δ(ppm) 7.64 (br m, 2 H), 3.69 (br, 2 H), 3.56 (br, 4 H), | | |
| | 3.41 (br t, 2 H), 3.13 (m, 2 H), 2.87 (m, 2 H), 1.65 (4 H), 1.53 (br, 2 H), 1.46 (s, 9 H). | | |
| Example 21.16 | 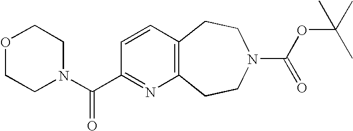 | tert-butyl 2- (morpholine-4- carbonyl)-5,6,8,9- tetrahydro- pyrido[2,3- d]azepine-7- carboxylate | 94 mg, 51% |

| | |
|---|---|
| NMR | ¹H NMR (300 MHz, CDCl₃): δ(ppm) 7.43 (br m, 2 H), 3.77 (s, 4 H), 3.66 (s, 4 H), |
| | 3.57 (br, 4 H), 3.13 (m, 2 H), 2.88 (m, 2 H), 1.47 (s, 9 H). |

### Example 21.17

### tert-Butyl 2-tert-butyl-5,6,8,9-tetrahydro-7H-pyrido[2,3-d]azepine-7-carboxylate

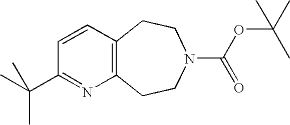

Tert-butyl lithium (1.93 mL, 3.28 mmol) was added to a suspension of copper cyanide (0.158 g, 1.77 mmol) in tetrahydrofuran (4.0 mL) under nitrogen at −40° C. A solution of the title compound from Example 17.1 (0.200 g, 0.505 mmol) in tetrahydrofuran (2.0 mL) was added slowly to the reaction mixture. The reaction was stirred at −40° C. for 24 hours and then warmed to r.t. over 6 hours. The reaction mixture was quenched with saturated ammonium chloride (20 mL) and diluted with ethyl acetate. The aqueous phase was extracted with ethyl acetate and the organic layer was washed with brine, dried with magnesium sulfate, filtered and concentrated. The product was purified by column chromatograph to give the title compound (101 mg, 66%). ¹H NMR (300 MHz, CDCl₃): δ(ppm) 7.29 (d, 1H), 7.07 (d, 1H), 3.57 (brt, 4H), 3.13 (m, 2H), 2.82 (br, 2H), 1.49 (s, 9H), 1.33 (s, 9H).

### Example 21.18

### tert-Butyl e 2-Dibutylamino-5,6,8,9-tetrahydro-pyrido[2,3-d]azepine-7-carboxylate ZH3968.062.1

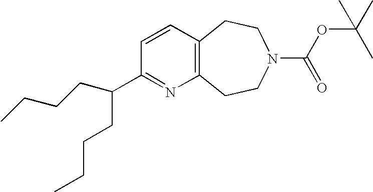

The title compound from Example 17.1 (0.10 g, 0.25 mmol) and dibutyl amine (0.5 mL) dissolved in dimethyl sulfoxide (2 mL) were heated to 120-150° C. in a microwave for 30 minutes. The reaction mixture was diluted with water and extracted with ethyl acetate, dried and concentrated. The product was purified by column chromatography to give the title compound (34 mg, 36%). ¹H NMR (300 MHz, CDCl₃): δ(ppm) 7.12 (d, 1H), 6.18 (d, 1H), 3.56 (br m, 4H), 3.44 (t, 4H), 2.97 (m, 2H), 2.71 (br, 2H), 1.55 (m, 6H), 1.50 (s, 9H), 1.36 (m, 5H), 0.94 (t, 7H).

Using the above general procedure, the following compounds were synthesized:

| Example | Structure | Name | Yield |
|---|---|---|---|
| Example 21.19 | 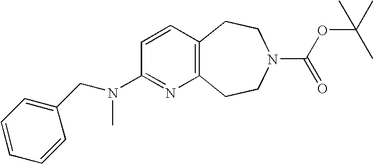 | tert-butyl 2-(benzyl- methyl-amino)- 5,6,8,9-tetrahydro- pyrido[2,3- d]azepine-7- carboxylate | 62 mg, 67% |
| NMR | ¹H NMR (300 MHz, CDCl₃): δ(ppm) 7.27 (m, 5 H), 7.18 (d, 1 H) | | |
| | 6.27 (d, 1 H), 4.81 (s, 2 H), 3.56 (br m, 4 H), 3.04 (s, 5 H), 2.75 (br, 2 H), 1.51 (s, 9 H). | | |
| Example 21.20 | 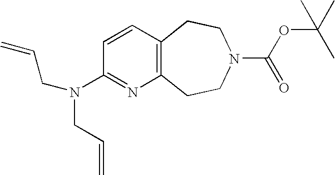 | tert-butyl 2- aiallylamino- 5,6,8,9-tetrahydro- pyrido[2,3- d]azepine-7- carboxylate | 20 mg, 23% |
| NMR | ¹H NMR (300 MHz, CDCl₃): δ(ppm) 7.15 (d, 1 H), 6.23 (d, 1 H), | | |
| | 5.86 (m, 2 H), 5.15 (m, 4 H), 4.10 (d, 4 H), 3.54 (br, 4 H), 2.99 (br t, 2 H), 2.72 (br s, 2 H), 1.50 (s, 9 H). | | |
| Example 21.21 | 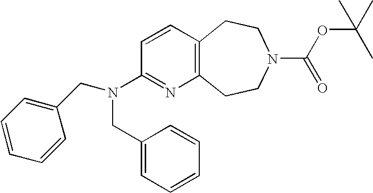 | tert-butyl 2- dibenzylamino- 5,6,8,9-tetrahydro- pyrido[2,3- d]azepine-7- carboxylate | 8 mg, 7% |
| NMR | ¹H NMR (300 MHz, CDCl₃): δ(ppm) 7.34 (m, 10 H), 7.12 (d, | | |
| | 1 H), 6.21 (d, 1 H), 4.78 (s, 4 H), 3.56 (br m, 4 H), 3.03 (br s, 2 H), 2.74 (br s, 2 H), 1.50 (s, 9 H). | | |
| Example 21.22 | 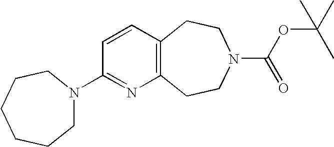 | tert-butyl 2-azepan- 1-yl-5,6,8,9- tetrahydro- pyrido[2,3-d]azepine- 7-carboxylate | 32 mg, 73% |
| NMR | ¹H NMR (300 MHz, CDCl₃): δ(ppm) 7.13 (d, 1 H), 6.22 (d, 1 H), | | |
| | 3.58 (m, 8 H), 2.97 (m, 2 H), 2.70 (m, 2 H), 1.74 (m, 4 H), 1.54 (m, 4 H), 1.49 (s, 9 H). | | |
| Example 21.23 | 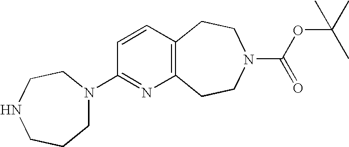 | tert-butyl 2- [1,4]diazepan-1-yl- 5,6,8,9-tetrahydro- pyrido[2,3- d]azepine-7- carboxylate | 28 mg, 65% |
| NMR | ¹H NMR (300 MHz, CDCl₃): δ(ppm) 7.15 (d, 1 H), 6.24 (d, 1 H), | | |
| | 3.72 (m, 4 H), 3.45 (br m, 4 H), 2.99 (m, 3 H), 2.85 (m, 3 H), 2.70 (br s, 2 H), 1.85 (m, 2 H), 1.48 (m, 9 H). | | |
| Example 21.24 | 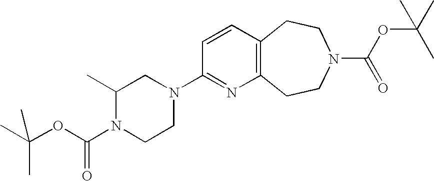 | tert-butyl 2-(4-tert- Butoxycarbonyl-3- methyl-piperazin-1- yl)-5,6,8,9- tetrahydro- pyrido[2,3- d]azepine-7- carboxylate | 11 mg, 10% |
| NMR | ¹H NMR (300 MHz, CDCl₃): δ(ppm) 7.22 (d, 1 H), 6.38 (d, 1 H), | | |
| | 4.33 (br, s, 1 H), 4.15 (d, 1 H), 3.92 (d, 2 H), 3.54 (br m, 4 H), 3.23 (m, 1 H), 3.10 (m, 1 H), 2.99 (br, 2 H), | | |
| | 2.86 (m, 1 H), 2.75 (br, 2 H), 1.49 (s, 18 H), 1.21 (d, 3 H). | | |
| Eample 21.25 | 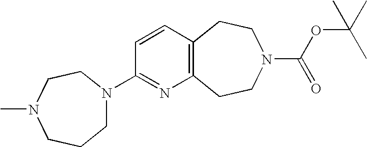 | tert-butyl 2-(4- Methyl- [1,4]diazepan-1-yl)- 5,6,7,8-tetrahydro- pyrido[2,3- d]azepine-7- carboxylate | 26 mg, 56% |
| NMR | ¹H NMR (300 MHz, CDCl₃): δ(ppm) 7.16 (d, 1 H), 6.11 (d, 1 H), 3.83 (m, 2 H), | | |
| | 3.57 (m, 6 H), 2.97 (m, 2 H), 2.67 (m, 4 H), 2.56 (m, 2 H), 2.36 (m, 3 H), 2.00 (m, 2 H), 1.47 (m, 9 H). | | |
| Example 21.26 | 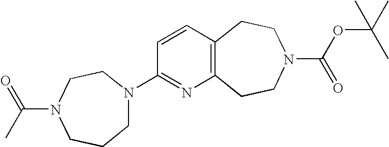 | tert-butyl 2-(4- Acetyl- [1,4]diazepan-1-yl)- 5,6,8,9-tetrahydro- pyrido[2,3- d]azepine-7- carboxylate | 34 mg, 69% |
| NMR | ¹H NMR (300 MHz, CDCl₃): δ(ppm) 7.16 (d, 1 H), 6.26 (m, 1 H), | | |
| | 3.87 (t, 1 H), 3.37-3.72 (br m, 11 H), 2.96 (br, 2 H), 3.72 (br, 2 H), 2.09 (s, 3 H), 1.96 (m, 2 H), 1.48 (m, 9 H). | | |
| Example 21.27 | 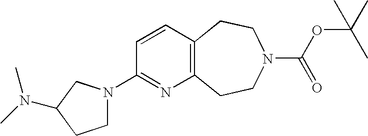 | tert-butyl 2-(3- dimethylamino- pyrrolidin-1-yl)- 5,6,8,9-tetrahydro- pyrido[2,3- d]azepine-7- carboxylate | 26 mg, 58% |
| NMR | ¹H NMR (300 MHz, CDCl₃): δ(ppm) 7.17 (d, 1 H), 6.10 (d, 1 H), 3.76 (m, 1 H), | | |
| | 3.57 (m, 6 H), 3.36 (m, 1 H), 3.18 (t, 1 H), 2.99 (br, 2 H), 2.74 (br, 3 H), 2.31 (s, 6 H), 1.47 (m, 9 H). | | |
| Example 21.28 | 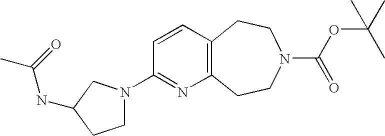 | tert-butyl 2-(3- acetylamino- pyrrolidin-1-yl)- 5,6,8,9-tetrahydro- pyrido[2,3- d]azepine-7- carboxylate | 37 mg, 78% |
| NMR | ¹H NMR (300 MHz, CDCl₃): δ(ppm) 7.17 (d, 1 H), 6.11 (d,, 1 H) 6.10 (br, 1 H), | | |
| | 4.56 (m, 1 H), 3.46-3.68 (m, 9 H), 2.96 (m, 2 H), 2.70 (m, 2 H), 2.28 (m, 1 H), 1.96 (m, 4 H), 1.47 (s, 9 H). | | |
| Example 21.29 | 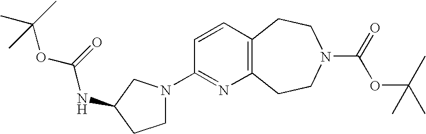 | (3R)-tert-butyl 2-(3- tert- butoxycarbonyl- amino- pyrrolidin-1-yl)- 5,6,8,9-tetrahydro- pyrido[2,3- d]azepine-7- carboxylate | 41.4 mg, 76% |
| NMR | ¹H NMR (300 MHz, CDCl₃): δ(ppm) ¹H NMR (300 MHz, CDCl₃): δ(ppm) 7.18 (d, | | |
| | 1 H), 6.12 (d,, 1 H) 4.76 (br, 1 H), 4.31 (m, 1 H), 3.50 (m, 8 H), 2.97 (m, 2 H), 2.72 (m, 2 H), 2.28 (m, | | |
| | 1 H), 1.95 (m, 1 H), 1.48 (s, 9 H), 1.45 (s, 9 H). | | |
| Example 21.30 | 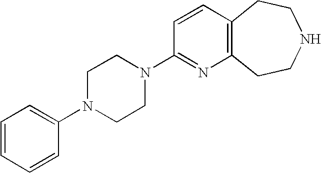 | tert-butyl 2-(4- phenylpiperazin-1- yl)-6,7,8,9- tetrahydro-5H- pyrido[2,3- d]azepine-7- carboxylate | 30.2 mg, 59% |
| NMR | ¹H NMR (300 MHz, CDCl₃): δ(ppm) ¹H NMR (300 MHz, CDCl₃): δ(ppm) 7.29 (m, | | |
| | 3 H), 7.00 (d, 2 H), 6.90 (t, 1 H), 6.48 (d, 1 H), 3.69 (m, 4 H), 3.57 (m, 4 H), 3.31 (m, 4 H), 3.05 (m, 2 H), | | |
| | 2.77 (m, 2 H), 1.50 (s, 9 H). | | |
| Example 21.31 | 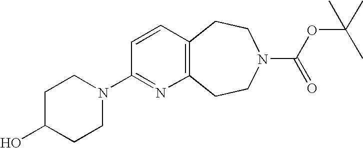 | tert-butyl 2-(4- Hydroxy-piperidin- 1-yl)-5,6,8,9- tetrahydro- pyrido[2,3- d]azepine-7- carboxylate | 31 mg, 71% |
| NMR | ¹H NMR (300 MHz, CDCl₃): δ(ppm) 7.20 (d, 1 H), 6.42 (d, 1 H), 4.405 (m, 2 H), 3.87 (m, | | |
| | 1 H), 3.54 (br, 4 H), 3.03 (br m, 4 H), 2.73 (br, 2 H), 1.97 (m, 2 H), 1.94 (s, 1 H), 1.60 (m, 2 H), 1.49 (s, 9 H). | | |
| Example 21.32 | 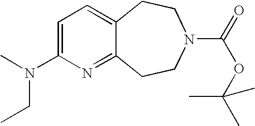 | tert-butyl 2- [ethyl(methyl)amino]- 5,6,8,9- tetrahydro-7H- pyrido[2,3- d]azepine-7- carboxylate | 63 mg, 84% |
| NMR | ¹H NMR (300 MHz, CDCl₃): δ(ppm) 7.16 (d, 1 H), 6.25 (d, 1 H), | | |
| | 3.56 (m, 6 H), 3.00 (s, m, 5 H), 2.72 (m, 2 H), 1.49 (s, 9 H), 1.21 (t, 3 H) | | |
| Example 21.33 | 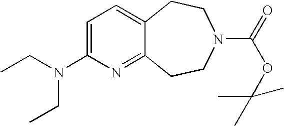 | tert-butyl 2- (diethylamino)- 5,6,8,9-tetrahydro- 7H-pyrido[2,3- d]azepine-7- carboxylate | 13 mg, 16% |
| NMR | ¹H NMR (300 MHz, CDCl₃): δ(ppm) 7.15 (d, 1 H), 6.23 (d, 1 H), | | |
| | 3.51 (m, 8 H), 3.99 (m, 2 H), 2.72 (m, 2 H), 1.49 (s, 9 H), 1.32 (t, 6 H) | | |
| Example 21.34 | 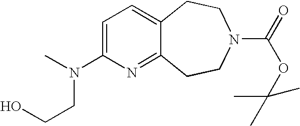 | tert-butyl 2-[(2- hydroxyethyl) (methyl)amino]- 5,6,8,9- tetrahydro-7H- pyrido[2,3- d]azepine-7- carboxylate | 43 mg, 54% |
| NMR | ¹H NMR (300 MHz, CDCl₃): δ(ppm) 7.25 (d, 1 H), 6.32 (d, | | |
| | 1 H), 6.07 (br, 1 H), 3.85 (t, m2H), 3.60 (m, 6 H), 3.04 (s, 2 H), 2.96 (m, 2 H), 2.75 (m, 2 H), 1.49 (s, 9 H). | | |
| Example 21.35 | 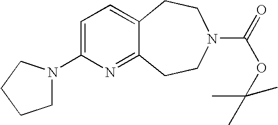 | tert-butyl 2- pyrrolidin-1-yl- 5,6,8,9-tetrahydro- 7H-pyrido[2,3- d]azepine-7- carboxylate | 71 mg, 91% |
| NMR | ¹H NMR (300 MHz, CDCl₃): δ(ppm) 7.17 (d, 1 H), 6.13 (d, 1 H), | | |
| | 3.50 (m, 8 H), 3.00 (m, 2 H), 2.74 (m, 2 H), 1.99 (m, 4 H), 1.49 (s, 9 H). | | |
| Example 21.36 | 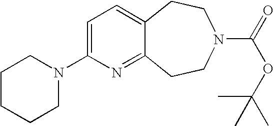 | Tert-butyl 2- piperidin-1-yl- 5,6,8,9-tetrahydro- 7H-pyrido[2,3- d]azepine-7- carboxylate | 070 mg, 91% |
| NMR | ¹H NMR (300 MHz, CDCl₃): δ(ppm) 7.20 (d, 1 H), 6.41 (d, 1 H), | | |
| | 3.51 (m, 8 H), 3.00 (m, 2 H), 2.73 (m, 2 H), 1.63 (m, 6 H), 1.49 (s, 9 H). | | |
| Example 21.37 | 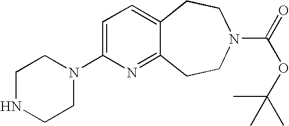 | Tert-bytyl 2- piperazin-1-yl- 5,6,8,9-tetrahydro- 7H-pyrido[2,3- d]azepine-7- carboxylate | 80 mg, 100% |
| NMR | ¹H NMR (300 MHz, CDCl₃): δ(ppm) 7.23 (d, 1 H), 6.42 (d, 1 H), | | |
| | 3.45 (m, 8 H), 3.00 (m, 6 H), 2.76 (m, 2 H), 1.87 (br, 1 H), 1.49 (s, 9 H). | | |
| Example 21.38 | 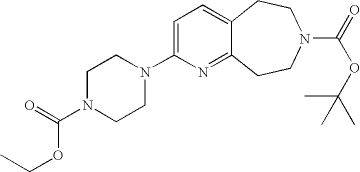 | Tert-bytyl 2-(4-tert- butoxycarbonyl- piperazin-1-yl)- 5,6,8,9-tetrahydro- 7H-pyrido[2,3- d]azepine-7- carboxylate | 16.7 mg, 33% |
| NMR | ¹H NMR (300 MHz, CDCl₃): δ(ppm) 7.25 (d, 1 H), 6.40 (d, 1 H), | | |
| | 4.17 (q, 2 H), 3.55 (m, 12 H), 3.00 (m, 2 H), 2.75 (m, 2 H), 1.49 (s, 9 H), 1.29 (t, 3 H). | | |
| Example 21.39 | 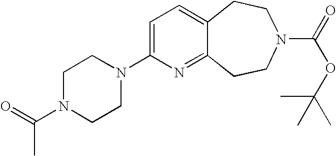 | Tert-bytyl 2-(4- acetyl-piperazin-1- yl)- 5,6,8,9-tetrahydro- 7H-pyrido[2,3- d]azepine-7- carboxylate | 17.8 mg, 38% |
| NMR | ¹H NMR (300 MHz, CDCl₃): δ(ppm) 7.25 (d, 1 H), 6.41 (d, 1 H), | | |
| | 3.75 (m, 2 H), 3.57 (m, 8 H), 3.44 (m, 2 H), 3.00 (m, 2 H), 2.76 (m, 2 H), 2.15 (s, 3 H), 1.49 (s, 9 H). | | |
| Example 21.40 | 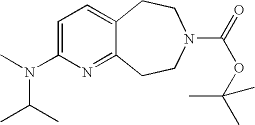 | Tert-bytyl 2- [isopropyl (methyl) amino]-5,6,8,9- tetrahydro-7H- pyrido[2,3- d]azepine-7- carboxylate | 33.9 mg, 42% |
| NMR | ¹H NMR (300 MHz, CDCl₃): δ(ppm) 7.17 (d, 1 H), 6.25 (d, 1 H), | | |
| | 4.84 (m, 1 H), 3.55 (m, 4 H), 3.00 (m, 2 H), 2.82 (s, 3 H), 2.73 (m, 2 H), 1.49 (s, 9 H), 1.15 (d, 6 H). | | |
| Example 21.41 | 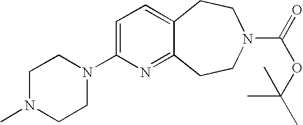 | Tert-bytyl 2-(4- methylpiperazin-1- yl)-5,6,8,9- tetrahydro-7H- pyrido[2,3- d]azepine-7- carboxylate | 20 mg 24% |
| NMR | ¹H NMR (300 MHz, CDCl₃): δ(ppm) 7.23 (d, 1 H), 6.41 (d, 1 H), | | |
| | 3.46 (m, 8 H), 3.00 (m, 2 H), 2.75 (m, 2 H), 2.53 (m, 4 H), 2.35 (s, 3 H), 1.49 (s, 9 H). | | |
| Example 21.42 | 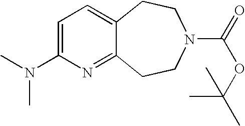 | tert-butyl 2- (dimethylamino)- 5,6,8,9-tetrahydro- 7H-pyrido[2,3- d]azepine-7- carboxylate | 40 mg, 54% |
| NMR | ¹H NMR (300 MHz, CDCl₃): δ(ppm) 7.19 (d, 1 H), 6.28 (d, 1 H), | | |
| | 3.55 (m, 4 H), 3.05 (s, 6 H), 2.98 (m, 2 H), 2.73 (m, 2 H), 1.50 (s, 9 H). | | |
| Example 21.43 | 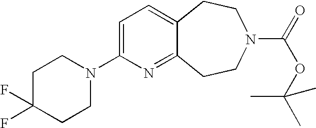 | Tert-butyl 2-(4,4- difluoropiperidin-1- yl)-5,6,8,9- tetrahydro-7H- pyrido[2,3- d]azepine-7- carboxylate | 39.1 mg, 84% |
| NMR | ¹H NMR (300 MHz, CDCl₃): δ(ppm) 7.24 (d, 1 H), 6.46 (d, 1 H), | | |
| | 3.70 (m, 4 H), 3.56 (m, 4 H), 3.00 (m, 2 H), 2.75 (m, 2 H), 2.00 (m, 4 H), 1.50 (s, 9 H). | | |
| Example 21.44 | 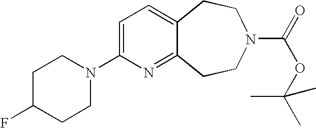 | Tert-butyl 2-(4- fluoropiperidin-1- yl)-5,6,8,9- tetrahydro-7H- pyrido[2,3- d]azepine-7- carboxylate | 23.9 mg, 54% |
| NMR | ¹H NMR (300 MHz, CDCl₃): δ(ppm) 7.22 (d, 1 H), 6.44 (d, 1 H), | | |
| | 4.83 (dm, 1 H), 3.56 (m, 8 H), 3.00 (m, 2 H), 2.74 (m, 2 H), 1.93 (m, 4 H), 1.50 (s, 9 H). | | |
| Example 21.45 | 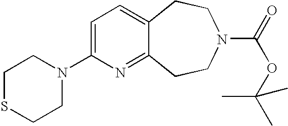 | Tert-butyl 2- thiomorpholin-4-yl- 5,6,8,9-tetrahydro- 7H-pyrido[2,3- d]azepine-7- carboxylate | 32.2 mg, 73% |
| NMR | ¹H NMR (300 MHz, CDCl₃): δ(ppm) 7.22 (d, 1 H), 6.37 (d, 1 H), | | |
| | 3.91 (m, 4 H), 3.55 (m, 4 H), 2.98 (m, 2 H), 2.74 (m, 2 H), 2.66 (m, 4 H), 1.49 (s, 9 H). | | |
| Example 21.46 | 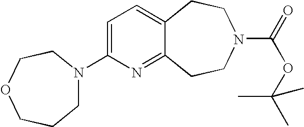 | Tert-butyl 2-(1,4- oxazepan-4-yl)- 5,6,8,9-tetrahydro- 7H-pyrido[2,3- d]azepine-7- carboxylate | 27.3 mg, 62% |
| NMR | ¹H NMR (300 MHz, CDCl₃): δ(ppm) 7.18 (d, 1 H), 6.27 (d, 1 H), | | |
| | 3.83 (m, 4 H), 3.70 (m, 4 H), 3.55 (m, 4 H), 2.97 (m, 2 H), 2.72 (m, 2 H), 2.00 (m, 2 H), 1.49 (s, 9 H). | | |
| Example 21.47 | 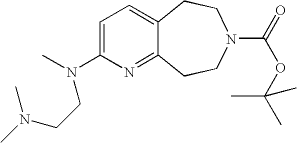 | Tert-butyl 2-[[2- (dimethylamino) ethyl](methyl)amino]- 5,6,8,9-tetrahydro- 7H-pyrido[2,3- d]azepine-7- carboxylate | 36.1 mg, 82% |
| NMR | ¹H NMR (300 MHz, CDCl₃): δ(ppm) 7.18 (d, 1 H), 6.25 (d, 1 H), 3.70 (t, 2 H), 3.53 (m, 4 H), | | |
| | 3.01 (s, 3 H), 2.97 (m, 2 H), 2.71 (m, 2 H), 2.00 (m, 2 H), 2.55 (t, 2 H), 2.36 (s, 6 H), 1.49 (s, 9 H). | | |
| Example 21.48 | 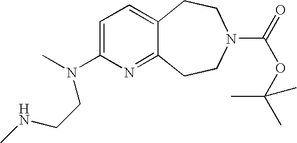 | Tert-butyl 2- {methyl[2- (methylamino)ethyl] amino}-5,6,8,9- tetrahydro-7H- pyrido[2,3- d]azepine-7- carboxylate | 35.5 mg, 84% |
| NMR | ¹H NMR (300 MHz, CDCl₃): δ(ppm) 7.19 (d, 1 H), 6.29 (d, 1 H), 3.68 (t, 2 H), 3.52 (m, 4 H), | | |
| | 3.13 (br, 1 H), 3.02 (s, 3 H), 2.95 (m, 2 H), 2.86 (t, 2 H), 2.71 (m, 2 H), 2.51 (s, 3 H), 1.48 (s, 9 H). | | |
| Example 21.49 | 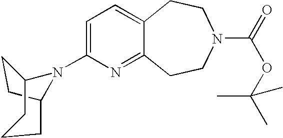 | Tert-butyl 2-(8- azabicyclo[3.2.1]oct- 8-yl)-5,6,8,9- tetrahydro-7H- pyrido[2,3- d]azepine-7- carboxylate | 9 mg, 20% |
| NMR | ¹H NMR (300 MHz, CDCl₃): δ(ppm) 7.17 (d, 1 H), 6.31 (d, 1 H), 4.45 (m, 2 H), | | |
| | 3.55 (m, 4 H), 2.99 (m, 2 H), 2.75 (m, 2 H), 2.08 (m, 2 H), 1.83 (m, 5 H), 1.50 (s & m, 10 H), 1.36 (m, 2 H). | | |
| Example 21.50 | 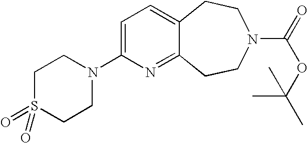 | Tert-butyl 2-(1,1- dioxidothio- morpholin- 4-yl)-5,6,8,9- tetrahydro-7H- pyrido[2,3- d]azepine-7- carboxylate | 10 mg, 21% |
| NMR | ¹H NMR (300 MHz, CDCl₃): δ(ppm) 7.29 (d, 1 H), 6.62 (d, 1 H), | | |
| | 4.14 (m, 4 H), 3.57 (m, 4 H), 3.03 (m, 6 H), 2.78 (m, 2 H), 1.50 (s, 9 H). | | |
| Example 21.51 | 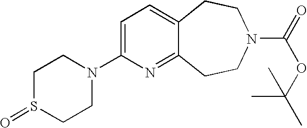 | Tert-butyl 2-(1- oxidothiomorpholin- 4-yl)-5,6,8,9- tetrahydro-7H- pyrido[2,3- d]azepine-7- carboxylate | 10 mg, 21% |
| NMR | ¹H NMR (300 MHz, CDCl₃): δ(ppm) 7.29 (d, 1 H), 6.62 (d, 1 H), | | |
| | 4.14 (m, 4 H), 3.57 (m, 4 H), 3.03 (m, 6 H), 2.78 (m, 2 H), 1.50 (s, 9 H). | | |
| Example 21.52 | 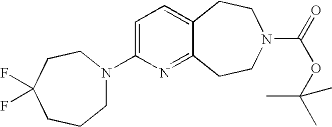 | Tert-butyl 2-(4,4- difluoroazepan-1- yl)-5,6,8,9- tetrahydro-7H- pyrido[2,3- d]azepine-7- carboxylate | 15.7 mg, 32% |
| NMR | ¹H NMR (300 MHz, CDCl₃): δ(ppm) 7.20 (d, 1 H), 6.25 (d, 1 H), | | |
| | 3.74 (m, 2 H), 3.59 (m, 6 H), 2.99 (m, 2 H), 2.74 (m, 2 H), 2.20 (m, 6 H), 1.50 (s, 9 H). | | |
| Example 21.57 | 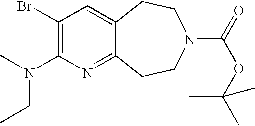 | Tert-butyl 3-bromo- 2- [ethyl (methyl)amino]- 5,6,8,9- tetrahydro-7H- pyrido[2,3- d]azepine-7- carboxylate | 60 mg, 89.6% |
| NMR | ¹H NMR (300 MHz, CDCl₃): δ(ppm) 7.49 (d, 1 H), 3.56 (m, 4 H), | | |
| | 3.29 (q, 2 H), 3.00 (m, 2 H), 2.89 (s, 3 H), 2.74 (m, 1 H), 1.49 (s, 9 H), 1.20 (t, 3 H). | | |
| Example 21.58 | 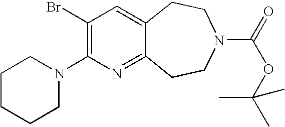 | Tert-butyl 3-bromo- 2-piperidin-1-yl 5,6,8,9-tetrahydro- 7H-pyrido[2,3- d]azepine-7- carboxylate | 54 mg, 75.5% |
| NMR | ¹H NMR (300 MHz, CDCl₃): δ(ppm) 7.49 (d, 1 H), 3.56 (m, 4 H), | | |
| | 3.21 (m, 4 H), 3.01 (m, 2 H), 2.75 (m, 2 H), 1.65 (m, 6 H), 1.49 (s, 9 H). | | |
| Example 21.59 | 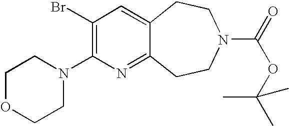 | Tert-butyl 3-bromo- 2-morpholin-4-yl 5,6,8,9-tetrahydro- 7H-pyrido[2,3- d]azepine-7- carboxylate | 66 mg, 92.3% |
| NMR | ¹H NMR (300 MHz, CDCl₃): δ(ppm) 7.53 (d, 1 H), 3.86 (m, 4 H), | | |
| | 3.57 (m, 4 H), 3.30 (m, 4 H), 3.02 (m, 2 H), 2.76 (m, 2 H), 1.50 (s, 9 H). | | |
| Example 21.60 | 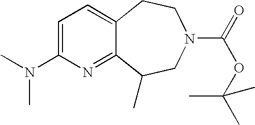 | Tert-butyl 2- dimethylamino-9- methyl-5,6,8,9- tetrahydro-7H- pyrido[2,3- d]azepine-7- carboxylate | 60 mg, 70% |
| NMR | ¹H NMR (300 MHz, CDCl₃): δ(ppm) 7.17 (d, 1 H), 6.27 (d, 1 H), | | |
| | 3.10-3.60 (m, 5 H), 3.06 (s, 6 H), 2.73 (m, 2 H), 1.49 (s, 9 H), 1.32 (m, 3 H), | | |
| Example 21.61 | 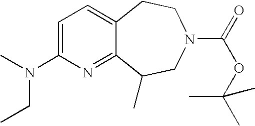 | Tert-butyl 2- [ethyl (methyl)amino]- 9-methyl-5,6,8,9- tetrahydro-7H- pyrido[2,3- d]azepine-7- carboxylate | 49 mg, 76.7% |
| NMR | ¹H NMR (300 MHz, CDCl₃): δ(ppm) 7.15 (d, 1 H), 6.23 (d, 1 H), | | |
| | 3.57 (m, 6 H), 3.00 (m, 1 H), 2.84 (s, 3 H), 2.70 (m, 2 H), 1.49 (s, 9 H), 1.31 (m, 3 H), 1.13 (t, 3 H). | | |
| Example 21.62 | 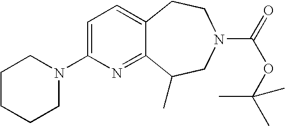 | Tert-butyl 9-methyl- 2-piperidin-1-yl- 5,6,8,9-tetrahydro- 7H-pyrido[2,3- d]azepine-7- carboxylate | 65 mg, 94% |
| NMR | ¹H NMR (300 MHz, CDCl₃): δ(ppm) 7.17 (d, 1 H), 6.39 (d, 1 H), | | |
| | 2.60-3.80 (m, 11 H), 1.64 (m, 6 H), 1.49 (s, 9 H), 1.31 (m, 3 H) | | |
| Example 21.63 | 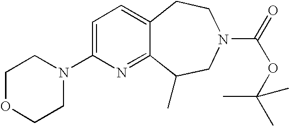 | Tert-butyl 9-methyl- 2-morpholin-4-yl- 5,6,8,9-tetrahydro- 7H-pyrido[2,3- d]azepine-7- carboxylate | 76 mg, 100% |
| NMR | ¹H NMR (300 MHz, CDCl₃): δ(ppm) 7.23 (d, 1 H), 6.38 (d, 1 H), | | |
| | 4.12 (m, 4 H), 3.53 (m, 8 H), 3.40 (m, 1 H), 2.80 (m, 2 H), 1.48 (s, 9 H), 1.30 (m, 3 H) | | |
| Example 21.64 | 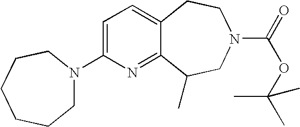 | Tert-butyl 2- azepan-1-yl-9- methyl-5,6,8,9- tetrahydro-7H- pyrido[2,3- d]azepine-7- carboxylate | 66 mg, 92% |
| NMR | ¹H NMR (300 MHz, CDCl₃): δ(ppm) 7.23 (d, 1 H), 6.38 (d, 1 H), | | |
| | 4.12 (m, 4 H), 3.53 (m, 8 H), 3.40 (m, 1 H), 2.80 (m, 2 H), 1.48 (s, 9 H), 1.30 (m, 3 H) | | |
| Example 21.65 | 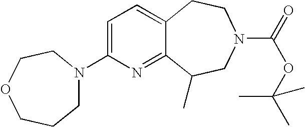 | Tert-butyl 2-(1,4- oxazepan-4-yl)-9- methyl-5,6,8,9- tetrahydro-7H- pyrido[2,3- d]azepine-7- carboxylate | 22 mg, 51% |
| NMR | ¹H NMR (300 MHz, CDCl₃): δ(ppm) 7.16 (d, 1 H), 6.26 (d, 1 H), | | |
| | 2.60-3.95 (m, 15 H), 2.03 (m, 2 H), 1.48 (s, 9 H), 1.30 (m, 3 H) | | |
| Example 21.66 | 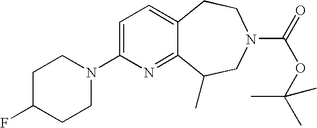 | Tert-butyl 2-(4- fluoropiperidin-1- yl)-9-methyl- 5,6,8,9-tetrahydro- 7H-pyrido[2,3- d]azepine-7- carboxylate | 21.7 mg, 50% |
| NMR | ¹H NMR (300 MHz, CDCl₃): δ(ppm) 7.20 (d, 1 H), 6.43 (d, 1 H), | | |
| | 4.84 (dm, 1 H), 3.56 (m, 8 H), 3.13 (m, 1 H), 2.80 (m, 2 H), 1.95 (m, 4 H), 1.49 (s, 9 H), 1.31 (m, 3 H) | | |
| Example 21.67 | 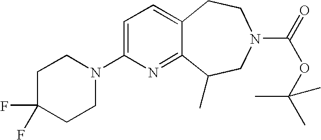 | Tert-butyl 2-(4,4- difluropiperidin-1- yl)-9-methyl- 5,6,8,9-tetrahydro- 7H-pyrido[2,3- d]azepine-7- carboxylate | 23.1 mg, 50% |
| NMR | ¹H NMR (300 MHz, CDCl₃): δ(ppm) 7.22 (d, 1 H), 6.46 (d, 1 H), | | |
| | 3.71 (m, 4 H), 3.38 (m, 4 H), 3.14 (m, 1 H), 2.75 (m, 2 H), 2.01 (m, 4 H), 1.48 (s, 9 H), 1.31 (m, 3 H) | | |
| Example 21.68 | 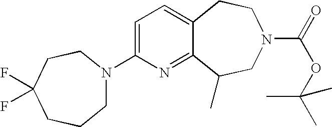 | Tert-butyl 2-(4,4- difluoroazepan-1- yl)-9-methyl- 5,6,8,9-tetrahydro- 7H-pyrido[2,3- d]azepine-7- carboxylate | 39 mg, 41.3% |
| NMR | ¹H NMR (300 MHz, CDCl₃): δ(ppm) 7.18 (d, 1 H), 6.24 (d, 1 H), | | |
| | 2.60-3.80 (m, 11 H), 2.10 (m, 6 H), 1.48 (s, 9 H), 1.30 (m, 3 H) | | |
| Example 21.69 | 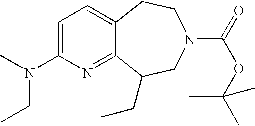 | Tert-butyl 2- [ethyl (methyl)amino- 9-methyl-2- 5,6,8,9-tetrahydro- 7H-pyrido[2,3- d]azepine-7- carboxylate | 32 mg, 44% |
| NMR | ¹H NMR (300 MHz, CDCl₃): δ(ppm) 7.15 (d, 1 H), 6.23 (d, 1 H), 3.57 (m, 6 H), 3.00 (s, 3 H), | | |
| | 2.87 (m, 2 H), 2.66 (m, 1 H), 1.90 (m, 1 H), 1.68 (m, 1 H), 1.48 (s, 9 H), 1.12 (t, 3 H), 0.99 (t, 3 H). | | |
| Example 21.70 | 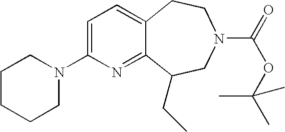 | Tert-butyl 9-ethyl-2- piperidin-1-yl- 5,6,8,9-tetrahydro- 7H-pyrido[2,3- d]azepine-7- carboxylate | 32 mg, 40% |
| NMR | ¹H NMR (300 MHz, CDCl₃): δ(ppm) 7.17 (d, 1 H), 6.39 (d, 1 H), | | |
| | 3.30-3.80 (m, 8 H), 2.88 (m, 2 H), 2.65 (m, 1 H), 1.87 (m, 1 H), 1.63 (br, 7 H), 1.49 (s, 9 H), 0.98 (t, 3 H) | | |
| Example 21.71 | 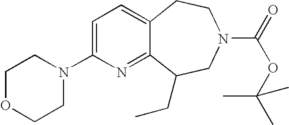 | Tert-butyl 9-ethyl-2- morpholin-4-yl- 5,6,8,9-tetrahydro- 7H-pyrido[2,3- d]azepine-7- carboxylate | 71 mg,, 90% |
| NMR | ¹H NMR (300 MHz, CDCl₃): δ(ppm) 7.23 (d, 1 H), 6.39 (d, 1 H), 3.83 (m, 4 H), | | |
| | 3..32-3.76 (m, 8 H), 2.89 (m, 2 H), 2.71 (m, 1 H), 1.89 (m, 1 H), 1.68 (m, 1 H), 1.48 (s, 9 H), 0.98 (t, 3 H) | | |
| Example 21.72 | 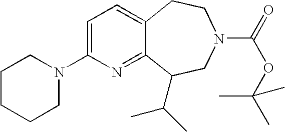 | Tert-butyl 9- isopropyl-2- piperidin-1-yl- 5,6,8,9-tetrahydro- 7H-pyrido[2,3- d]azepine-7- carboxylate | 41 mg, 54.9% |
| NMR | ¹H NMR (300 MHz, CDCl₃): δ(ppm) 7.15 (d, 1 H), 6.40 (d, 1 H), 3.75 (m, 2 H), | | |
| | 3.48 (m, 5 H), 3.26 (m, 1 H), 2.20-3.10 (m, 4 H), 1.63 (bs, 6 H), 1.48 (s, 9 H), 1.07 (d, 3 H), 0.81 (d, 3 H). | | |
| Example 21.73 | 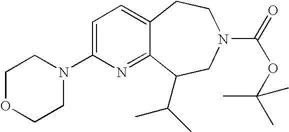 | Tert-butyl 9- isopropyl-2- morpholin-4-yl- 5,6,8,9-tetrahydro- 7H-pyrido[2,3- d]azepine-7- carboxylate | 55 mg, 73% |
| NMR | ¹H NMR (300 MHz, CDCl₃): δ(ppm) 7.21 (d, 1 H), 6.38 (d, 1 H), | | |
| | 2.20-3.90 (m, 16 H), 1.47 (s, 9 H), 1.07 (d, 3 H), 0.81 (d, 3 H). | | |
| Example 21.74 | 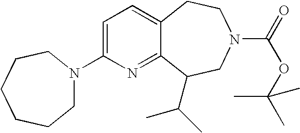 | Tert-butyl 2- azepan-1-yl-9- isoprppyl-5,6,8,9- tetrahydro-7H- pyrido[2,3 d]azepine-7- carboxylate | 69 mg, 89% |

| | |
|---|---|
| NMR | ¹H NMR (300 MHz, CDCl₃): δ(ppm) 7.10 (d, 1 H), 6.21 (d, 1 H), 3.65 (m, 7 H), |
| | 3.45 (m, 1 H), 2.30-3.00 (m, 4 H), 1.76 (m, 4 H), 1.52 (m, 4 H), 1.47 (s, 9 H), 1.07 (d, 3 H), 0.84 (d, 3 H). |

### Example 21.75

### tert-Butyl 2-(methylamino)-5,6,8,9-tetrahydro-7H-pyrido[2,3-d]azepine-7-carboxylate

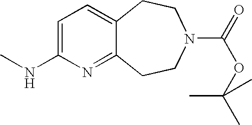

tert-butyl 2-(benzyl-methyl-amino)-5,6,8,9-tetrahydro-pyrido[2,3-d]azepine-7-carboxylate (98.5 mg, 0.27 mmol) was mixed with 20% Pd(OH)₂ (20 mg) in methanol (2 mL) and stirred under H₂ overnight. The reaction mixture was filtered and purified by column chromatography to give the title compound 35.4 mg (47%)

### Example 21.76

### tert-Butyl 2-[acetyl(methyl)amino]-5,6,8,9-tetrahydro-7H-pyrido[2,3-d]azepine-7-carboxylate

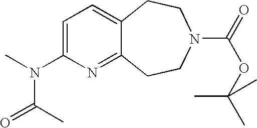

The title compound (68 mg, 53.3%) was prepared from tert-butyl 2-(methylamino)-5,6,8,9-tetrahydro-7H-pyrido[2,3-d]azepine-7-carboxylate (110.6 mg, 0.4 mmol) reacted with acetyl chloride (37.3 μL, 0.53 mmol) and triethylamine (54 mg, 0.53 mmol) in dichloromethane (2 mL) at 0° C. ¹H NMR (300 MHz, CDCl₃): δ(ppm) 7.45 (d, 1H), 6.97 (br, 1H), 3.57 (m, 4H), 3.30 (s, 3H), 3.08 (m, 2H), 2.86 (m, 2H), 2.02 (s, 3H), 1.46 (s, 9H).

### Example 21.77

### tert-Butyl 2-(1-oxidothiomorpholin-4-yl)-5,6,8,9-tetrahydro-7H-pyrido[2,3-d]azepine-7-carboxylate

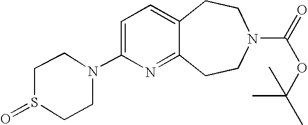

To a solution of NaIO₄ (132.6 mg, 0.62 mmol) in water (2 mL) at 0° C., tert-butyl 2-(thiomorpholin-4-yl)-5,6,8,9-tetrahydro-7H-pyrido[2,3-d]azepine-7-carboxylate (143.3 mg, 0.41 mmol) in methanol (4 mL) and DMF (4 mL) was added. the reaction mixture was stirred at 0° C. for 24 hours and then filtered. The filtrate was extracted with dichloromethane and concentrated. The residue was purified by column chromatography to give the title compound 116.9 mg (78%). ¹H NMR (300 MHz, CDCl₃): δ(ppm) 7.23 (d, 1H), 6.46 (d, 1H), 4.10 (m, 4H), 3.52 (m, 4H), 2.96 (m, 2H), 2.75 (m, 6H), 1.45 (s, 9H).

### Example 21.78

### Tert-butyl 3-chloro-2-piperidin-1-yl 5,6,8,9-tetrahydro-7H-pyrido[2,3-d]azepine-7-carboxylate

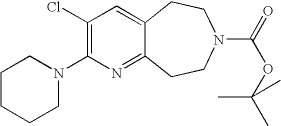

tert-Butyl 2-piperidin-1-yl 5,6,8,9-tetrahydro-7H-pyrido[2,3-d]azepine-7-carboxylate (50 mg, 0.153 mmol) was mixed with N-chlorosuccinimide (24 mg, 0.18 mmol) in acetonitrile (1.5 mL) and heated at 68° C. overnight. The reaction mixture was concentrated with silical gel and purified by column chromatography with 15% ethyl acetate in hexanes to give the title compound 50 mg (91%). ¹H NMR (300 MHz, CDCl₃): δ(ppm) 7.30 (s, 1H), 3.56 (m, 4H), 3.28 (m, 4H), 3.03 (m, 2H), 2.75 (m, 2H), 1.68 (m, 6H), 1.49 (s, 9H).

In a similar manner the following compounds were synthesized:

| Example | Structure | Name | Yield |
|---|---|---|---|
| Example 21.79 | 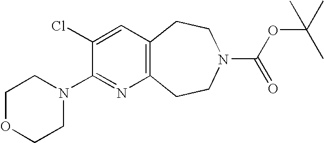 | Tert-Butyl 3-chloro- 2-morpholin-4-yl- 5,6,8,9-tetrahydro- 7H-pyrido[2,3- d]azepine-7- carboxylate | 43 mg, 77% |
| NMR | ¹H NMR (300 MHz, CDCl₃): δ(ppm) 7.34 (s, 1 H), 3.86 (m, 4 H), 3.85 | | |
| | (m, 4 H), 3.57 (m, 4 H), 3.32 (m, 4 H), 3.02 (m, 2 H), 2.77 (m, 2 H), 1.49 (s, 9 H). | | |
| Example 21.80 | 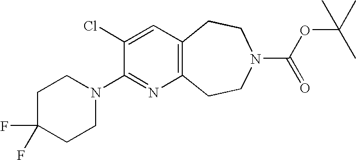 | Tert-Butyl 3-chloro- 2-(4,4- difluoropiperidin-1- yl)-5,6,8,9- tetrahydro-7H- pyrido[2,3- d]azepine-7- carboxylate | 15.3 mg, 56% |
| NMR | ¹H NMR (300 MHz, CDCl₃): δ(ppm) 7.34 (s, 1 H), 3.57 (m, 4 H), 3.40 | | |
| | (m, 4 H), 3.02 (m, 2 H), 2.77 (m, 2 H), 2.13 (m, 4 H), 1.49 (s, 9 H). | | |
| Example 21.81 | 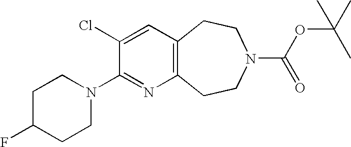 | Tert-Butyl 3-chloro- 2-(4- fluoropiperidin-1- yl)-5,6,8,9- tetrahydro-7H- pyrido[2,3- d]azepine-7- carboxylate | 25.3 mg, 89% |
| NMR | ¹H NMR (300 MHz, CDCl₃): δ(ppm) 7.32 (s, 1 H), 4.82 (dm, 1 H), 3.47 | | |
| | (m, 6 H), 3.25 (m, 2 H), 3.02 (m, 2 H), 2.76 (m, 2 H), 1.96 (m, 4 H), 1.49 (s, 9 H). | | |
| Example 21.82 | 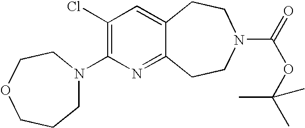 | Tert-butyl 3-chloro- 2-(1,4-oxazepan-4- yl)-5,6,8,9- tetrahydro-7H- pyrido[2,3- d]azepine-7- carboxylate | 17.9 mg, 60% |

| | |
|---|---|
| NMR | ¹H NMR (300 MHz, CDCl₃): δ(ppm) 7.27 (s, 1 H), 3.86 (m, 4 H), 3.70 |
| | (m, 4 H), 3.55 (m, 4 H), 2.97 (m, 2 H), 2.73 (m, 2 H), 2.05 (m, 2 H), 1.49 (s, 9 H). |

### Example 21.83

### (9R)- and (9S)-tert-butyl 2-[ethyl(methyl)amino]-9-methyl-5,6,8,9-tetrahydro-7H-pyrido[2,3-d]azepine-7-carboxylate

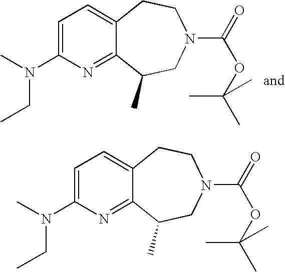

The (9R)-tert-butyl 2-[ethyl(methyl)amino]-9-methyl-5,6,8,9-tetrahydro-7H-pyrido[2,3-d]azepine-7-carboxylate and the (9S)-tert-butyl 2-[ethyl(methyl)amino]-9-methyl-5,6,8,9-tetrahydro-7H-pyrido[2,3-d]azepine-7-carboxylate (Rt=6.6 min, 36 mg; Rt=7.8 min, 35.1 mg) were separated from racemic tert-butyl 2-[ethyl(methyl)amino]-9-methyl-5,6,8,9-tetrahydro-7H-pyrido[2,3-d]azepine-7-carboxylate by Chiralcel OJ with 1% ethanol in hexanes.

### Example 21.84

### (9R)- and (9S)-tert-butyl 9-methyl-2-piperidin-1-yl-5,6,8,9-tetrahydro-7H-pyrido[2,3-d]azepine-7-carboxylate

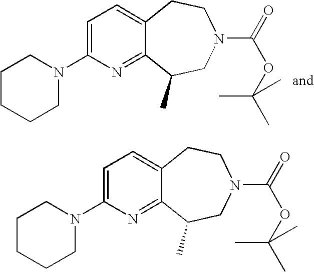

The (9R)-tert-butyl 9-methyl-2-piperidin-1-yl-5,6,8,9-tetrahydro-7H-pyrido[2,3-d]azepine-7-carboxylate and the (9S)-tert-butyl 9-methyl-2-piperidin-1-yl-5,6,8,9-tetrahydro-7H-pyrido[2,3-d]azepine-7-carboxylate.

(Rt=6.95 min, 42.5 mg,; Rt=8.56 min, 42 mg,) were separated from racemic tert-butyl 9-methyl-2-piperidin-1-yl-5,6,8,9-tetrahydro-7H-pyrido[2,3-d]azepine-7-carboxylate by Chiralcel OJ with 1% ethanol in hexanes.

### Example 21.85

### (9R)- and (9S)-tert-butyl 9-methyl-2-morpholin-4-yl-5,6,8,9-tetrahydro-7H-pyrido[2,3-d]azepine-7-carboxylate

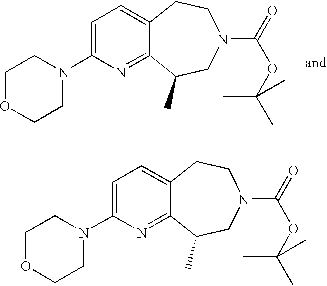

The (9R)-tert-butyl 9-methyl-2-morpholin-4-yl-5,6,8,9-tetrahydro-7H-pyrido[2,3-d]azepine-7-carboxylate and the (9S)-tert-butyl 9-methyl-2-morpholin-4-yl-5,6,8,9-tetrahydro-7H-pyrido[2,3-d]azepine-7-carboxylate (Rt=21.26 min, 48.2 mg,; Rt=17.4 min, 47.5 mg,) were separated from racemic tert-butyl 9-methyl-2-piperidin-1-yl-5,6,8,9-tetrahydro-7H-pyrido[2,3-d]azepine-7-carboxylate by Chiralcel OJ with 1% ethanol in hexanes.

### Example 21.86

### (9R)- and (9S)-2-(4-fluoropiperidin-1-yl)-9-methyl-5,6,8,9-tetrahydro-7H-pyrido[2,3-d]azepine-7-carboxylate

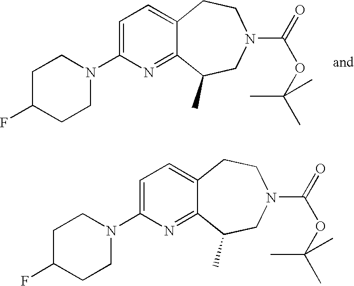

The (9R)-2-(4-fluoropiperidin-1-yl)-9-methyl-5,6,8,9-tetrahydro-7H-pyrido[2,3-d]azepine-7-carboxylate and the (S)-2-(4-fluoropiperidin-1-yl)-9-methyl-5,6,8,9-tetrahydro-7H-pyrido[2,3-d]azepine-7-carboxylate (Rt=7.51 min, 11 mg; Rt=9.68 min, 8.7 mg) were separated from racemic 2-(4-fluoropiperidin-1-yl)-9-methyl-5,6,8,9-tetrahydro-7H-pyrido[2,3-d]azepine-7-carboxylate by CHIRALPACK AD-H with 2.5% EtOH in Hexanes.

### Example 21.87

### (9R)- and (9S)-2-(4-fluoropiperidin-1-yl)-9-methyl-5,6,8,9-tetrahydro-7H-pyrido[2,3-d]azepine-7-carboxylate

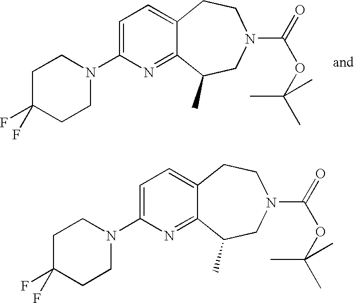

The (9R)-2-(4-fluoropiperidin-1-yl)-9-methyl-5,6,8,9-tetrahydro-7H-pyrido[2,3-d]azepine-7-carboxylate and the (S)-2-(4-fluoropiperidin-1-yl)-9-methyl-5,6,8,9-tetrahydro-7H-pyrido[2,3-d]azepine-7-carboxylate (Rt=5.12 min, 12.8 mg; Rt=6.62 min, 13 mg) were separated from 2-(4-fluoropiperidin-1-yl)-9-methyl-5,6,8,9-tetrahydro-7H-pyrido[2,3-d]azepine-7-carboxylate by CHIRALPACK AD-H with EtOH in Hexanes.

### Example 21.88

### (9R)- and (9S)-tert-butyl 9-methyl-2-(1,4-oxazepan-4-yl)-5,6,8,9-tetrahydro-7H-pyrido[2,3-d]azepine-7-carboxylate

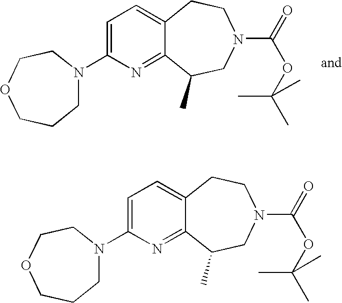

(9R)-tert-butyl 9-methyl-2-(1,4-oxazepan-4-yl)-5,6,8,9-tetrahydro-7H-pyrido[2,3-d]azepine-7-carboxylate and (9S)-tert-butyl 9-methyl-2-(1,4-oxazepan-4-yl)-5,6,8,9-tetrahydro-7H-pyrido[2,3-d]azepine-7-carboxylate (Rt=10.2 min, 10 mg; Rt=14.4 min, 10 mg) were separated from racemic tert-butyl 9-methyl-2-(1,4-oxazepan-4-yl)-5,6,8,9-tetrahydro-7H-pyrido[2,3-d]azepine-7-carboxylate by CHIRALPACK AD-H with 2.5% EtOH in Hexanes.

### Method A: (TFA-DCM)

### Example 22.1

### 6,7,8,9-Tetrahydro-5H-pyrido[2,3-d]azepine-2-carbonitrile

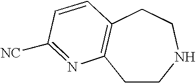

To a solution of tert-butyl 2-cyano-5,6,8,9-tetrahydro-pyrido[2,3-d]azepine-7-carboxylate (12 mg) in dichloromethane (1 mL) at 0° C., was added trifluoroacetic acid (0.5 mL). The reaction mixture was stirred at 0° C. for 2 hours. The reaction mixture was concentrated, diluted with ethyl acetate and washed with aqueous sodium carbonate. The organic layer was dried over sodium sulfate and concentrated to give the product. The product was treated with hydrochloric acid in diethyl ether to give the hydrochloric acid salt (2 salt equivalents, 5 mg).

### Method B: (HCl in Et₂O)

### Example 22.2

### azepine-2-carboxylic acid dimethylamide; dihydrochloride

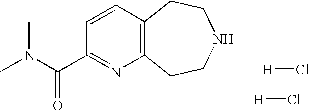

The title compound from Example 15 (0.040 g, 0.126 mmol) was dissolved in dichloromethane (2 mL) and 2M hydrochloric acid in diethyl ether (3 mL) for 6 hours. The reaction was concentrated to give the title compound as a hygroscopic brown solid (0.038 g, 100%). ¹H NMR (300 MHz, CDCl₃₊MeOD): δ(ppm) 8.48 (d, 1H), 8.01 (d, 1H), 3.74 (br, 2H), 3.59 (br, 2H), 3.52 (br, 4H), 3.14 (d, 6H).

In a similar manner the following compounds were synthesized:

| Example | Structure | Name | Method | Yield |
|---|---|---|---|---|
| Example 22.3 | 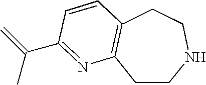 | 2-Isopropenyl- 6,7,8,9-tetrahydro-5H- pyrido[2,3-d]azepine | A | 17 mg, 55% |
| NMR | ¹H NMR (300 MHz, CDCl₃): δ(ppm): 7.35 (d, 1 H), 7.18 (d, 1 H), 5.88 (s, 1 H), | | | |
| | 5.23 (s, 1 H), 3.18 (m, 2 H), 3.01 (m, 4 H), 2.88 (m, 2 H), 2.19 (s, 3 H), 2.04 (br, 1 H). | | | |
| Example 22.4 | 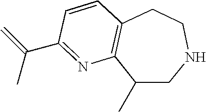 | 2-isopropenyl-9- methyl-6,7,8,9- tetrahydro-5H- pyrido[2,3-d]azepine | A | 45 mg, 76% |
| NMR | ¹H NMR (300 MHz, CDCl₃): δ(ppm): 7.35 (d, 1 H), 7.25 (d, 1 H), 5.95 (s, 1 H), | | | |
| | 5.26 (s, 1 H), 3.50 (m, 1 H), 3.20 (m, 3 H), 3.92 (m, 3 H), 2.21 (s, 3 H), 1.49 (d, 3 H). | | | |
| Example 22.5 | 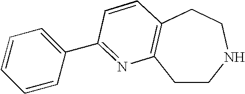 | 2-Phenyl-6,7,8,9- tetrahydro-5H- pyrido [2,3-d]azepine | A | 37 mg |
| NMR | ¹H NMR (300 MHz, CDCl₃): δ(ppm) 7.98 (m, 2 H), 7.49 (m, 5 H), 3.54 (m, 2 H), | | | |
| | 3.37 (m, 4 H), 3.14 (m, 2 H). | | | |
| Example 22.6 | 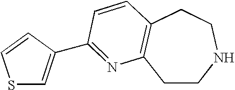 | 2-(3-thienyl)-6,7,8,9- tetrahydro-5H- pyrido[2,3-d]azepine | A | 77 mg, 100% |
| NMR | ¹H NMR (300 MHz, CDCl₃ + MeOD): δ(ppm) 7.88 (s, 1 H), 7.63 (d, 1 H), 7.40 | | | |
| | (m, 3 H), 3.39 (m, 2 H), 3.24 (m, 4 H), 3.08 (m, 2 H). | | | |
| Example 22.7 | 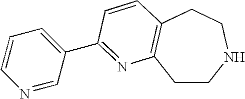 | 2-pyridin-3-yl-6,7,8,9- tetrahydro-5H- pyrido[2,3-d]azepine | A | 78 mg, 100% |
| NMR | ¹H NMR (300 MHz, CDCl₃ + MeOD): δ(ppm) 9.19 (s, 1 H), 8.57 (s, 1 H), 8.46 (d, | | | |
| | 1 H), 7.77 (m, 2 H), 7.55 (dd, 1 H), 3.32 (m, 8 H). | | | |
| Example 22.8 | 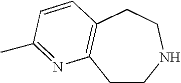 | 2-methyl-6,7,8,9- tetrahydro-5H- pyrid[2,3-d]azepine | A | 13 mg, 40% |
| NMR | ¹H NMR (300 MHz, CDCl₃): δ(ppm) 7.32 (d, 1 H), 6.96 (d, 1 H), 3.00-3.40 (m, 9 H), 2.51 (s, 3 H). | | | |
| Example 22.9 | 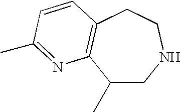 | 2,9-dimethyl-6,7,8,9- tetrahydro-5H- pyrido[2,3-d]azepine | A | 22 mg, 58% |
| NMR | ¹H NMR (300 MHz, CDCl₃): δ(ppm) 7.25 (d, 1 H), 6.89 (d, 1 H), 3.26 (m, 1 H), | | | |
| | 2.90 (m, 6 H), 2.50 (br, 4 H), 1.40 (s, 3 H). | | | |
| Example 22.10 | 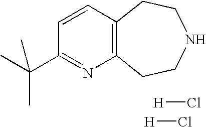 | 2-tert-butyl-6,7,8,9- tetrahydro-5H- pyrido[2,3-d]azepine dihydrochloride | B | 90.1 mg, 98% |
| NMR | ¹H NMR (300 MHz, CDCl₃): δ(ppm) 8.45 (d, 1 H), 7.94 (d, 1 H), 3.98 (m, 2 H), | | | |
| | 3.61 (m, 2 H), 3.49 (br, 4 H), 1.58 (s, 9 H). | | | |
| Example 22.11 | 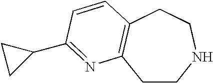 | 2-cyclopropyl- 6,7,8,9-tetrahydro- 5H-pyrido[2,3- d]azepine | A | 27 mg, 80% |
| NMR | ¹H NMR (300 MHz, CDCl₃ + MeOD): δ (ppm) 7.27 (d, 1 H), 6.87 (d, 1 H), 3.36 (m, | | | |
| | 2 H), 3.18 (m, 4 H), 3.00 (m, 2 H), 1.99 (m, 1 H), 0.95 (m, 4 H). | | | |
| Example 22.12 | 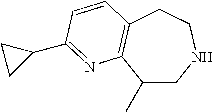 | 2-cyclopropyl-9- methyl-6,7,8,9- tetrahydro-5H- pyrido[2,3-d]azepine | A | 25 mg, 65% |
| NMR | ¹H NMR (300 MHz, CDCl₃): δ(ppm) 7.20 (d, 1 H), 6.84 (d, 1 H), 3.28 (m, 1 H), | | | |
| | 3.08 (m, 2 H), 2.82 (m, 4 H), 1.98 (m, 1 H), 1.35 (d, 3 H), 0.95 (m, 4 H). | | | |
| Example 22.13 | 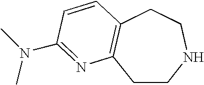 | N,N-dimethyl-6,7,8,9- tetrahydro-5H- pyrido[2,3-d]azepin- 2-amine | A | 24 mg, 62% |
| NMR | ¹H NMR (300 MHz, CDCl₃ + MeOD): δ(ppm) 7.19 (d, 1 H), 7.28 (d, 1 H), 3.02 (m, 12 H), 2.81 (m, 2 H). | | | |
| Example 22.14 | 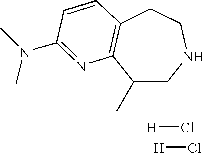 | N,N,9-trimethyl- 6,7,8,9-tetrahydro- 5H-pyrido[2,3- d]azepin-2-amine dihydrochloride | B | 45 mg, 78% |
| NMR | ¹H NMR (300 MHz, DMSO): δ(ppm) 9.95 (br, 1 H), 9.27 (br, 1 H), 7.72 (d, 1 H), | | | |
| | 6.92 (d, 1 H), 3.17 (m, 13 H), 1.38 (d, 3 H), 7.19 (d, 1 H), 7.28 (d, 1 H), 3.02 (m, 12 H), 2.81 (m, 2 H). | | | |
| Example 22.15 | 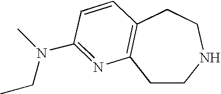 | N-ethyl-N-methyl- 6,7,8,9-tetrahydro- 5H-pyrido[2,3- d]azepin-2-amine | A | 56 mg, 98% |
| NMR | ¹H NMR (300 MHz, CDCl₃): δ (ppm) 7.19 (d, 1 H), 6.29 (d, 1 H), 3.57 (q, 2 H), | | | |
| | 3.24 (m, 6 H), 2.98 (m, 6 H), 1.13 (t, 3 H). | | | |
| Example 22.16 | 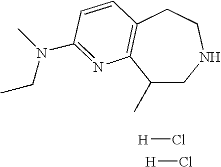 | N-ethyl-N,9-dimethyl- 6,7,8,9-tetrahydro- 5H-pyrido[2,3- d]azepin-2-amine dihydrochloride | B | 45 mg, 86% |
| NMR | ¹H NMR (300 MHz, MeOD): δ(ppm) 7.82 (d, 1 H), 7.14 (d, 1 H), 3.90 (m, 1 H), | | | |
| | 3.74 (q, 2 H), 3.56 (m, 3 H), 3.32 (s, 3 H), 3.20 (m, 3 H), 1.55 (d, 3 H), 1.29 (t, 3 H). | | | |
| Example 22.17 | 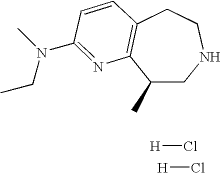 | (9R)-N-ethyl-N,9- dimethyl-6,7,8,9- tetrahydro-5H- pyrido[2,3-d]azepin- 2-amine dihydrochloride | B | 35 mg |
| Example 22.18 | 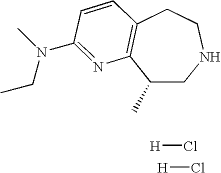 | (9S)-N-ethyl-N,9- dimethyl-6,7,8,9- tetrahydro-5H- pyrido[2,3-d]azepin- 2-amine dihydrochloride | B | 36 mg |
| Example 22.19 | 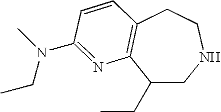 | N,9-diethyl-N-methyl- 6,7,8,9-tetrahydro- 5H-pyrido[2,3- d]azepin-2-amine | B | 34 mg, 100% |
| NMR | ¹H NMR (300 MHz, MeOD): δ(ppm) 7.84 (d, 1 H), 7.16 (d, 1 H), 3.00-3.85 (m, | | | |
| | 12 H), 1.96 (m, 2 H), 1.29 (t, 3 H), 1.08 (t, 3 H). | | | |
| Example 22.20 | 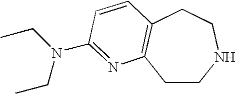 | N,N-diethyl-6,7,8,9- tetrahydro-5H- pyrido[2,3-d]azepin- 2-amine | A | 6.6 mg, 55% |
| NMR | ¹H NMR (300 MHz, CDCl₃): δ (ppm) 7.13 (d, 1 H), 6.21 (d, 1 H), 3.50 (q, 4 H), | | | |
| | 3.00 (m, 6 H), 2.76 (m, 2 H), 2.44 (br, 1 H), 1.15 (t, 6 H). | | | |
| Example 22.21 | 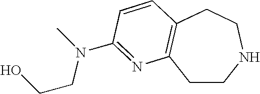 | 2-[methyl (6,7,8,9- tetrahydro-5H- pyrido[2,3-d]azepin- 2-yl)amino]ethanol | A | 15 mg, 38% |
| NMR | ¹H NMR (300 MHz, CDCl₃): δ (ppm) 7.22 (d, 1 H), 6.29 (d, 1 H), 3.83 (t, 2 H), | | | |
| | 3.67 (t, 2 H), 3.95 (m, 9 H), 2.76 (m, 2 H). | | | |
| Example 22.22 | 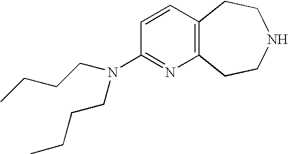 | Dibutyl-(6,7,8,9- tetrahydro-5H- pyrido[2,3-d]azepin- 2-yl)-amine | | 23 mg, 91% |
| NMR | ¹H NMR (300 MHz, CDCl₃): δ(ppm) 7.14 (d, 1 H), 6.19 (d, 1 H), 5.86 (br s, | | | |
| | 1 H), 3.42 (t, 4 H), 3.10 (m, 6 H), 2.85 (t, 2 H), 1.56 (m, 4 H), 1.30 (m, 4 H), 0.95 (t, 6 H). | | | |
| Example 22.23 | 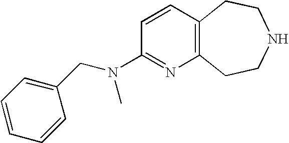 | Benzyl-methyl- (6,7,8,9-tetrahydro-5H- pyrido[2,3- d]azepin-2-yl)-amine | | 47 mg, 100% |
| NMR | ¹H NMR (300 MHz, CDCl₃): δ(ppm) 7.30 (m, 6 H), 6.78 (br s, 1 H), 6.29 (d, | | | |
| | 1 H), 4.81 (s, 2 H), 3.18 (m, 6 H), 3.04 (s, 3 H), 2.94 (s, 2 H). | | | |
| Example 22.24 | 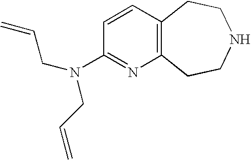 | Diallyl-(6,7,8,9- tetrahydro-5H- pyrido[2,3-d]azepin- 2-yl)-amine | | 20 mg, 100% |
| NMR | ¹H NMR (300 MHz, CDCl₃): δ(ppm) 7.17 (d, 1 H), 6.26 (d, 1 H), 5.86 (m, 2 H), | | | |
| | 5.16 (t, 4 H), 4.10 (d, 4 H), 3.17 (m, 6 H), 2.93 (m, 2 H). | | | |
| Example 22.25 | 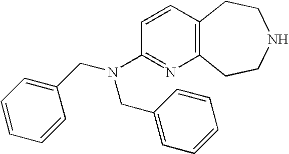 | Dibenzyl-(6,7,8,9- tetrahydro-5H- pyrido[2,3-d]azepin- 2-yl)-amine | | 7 mg, 100% |
| NMR | ¹H NMR (300 MHz, CDCl₃): δ(ppm) 7.34 (m, 10 H), 7.14 (d, 1 H), 6.25 (d, | | | |
| | 1 H), 4.79 (s, 4 H), 3.19 (m, 6 H), 3.00 (br, 2 H). | | | |
| Example 22.26 | 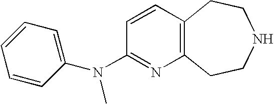 | Methyl-phenyl- (6,7,8,9-tetrahydro- 5H-pyrido[2,3- d]azepin-2-yl)-amine | | 13 mg, 89% |
| NMR | ¹H NMR (300 MHz, CDCl₃): δ(ppm) 7.39 (m, 2 H), 7.24 (m, 4 H), 7.07 (d, 1 H), | | | |
| | 6.35 (d, 1 H), 3.49 (s, 3 H), 3.23 (s, 4 H), 3.14 (m, 2 H), 2.93 (m, 2 H). | | | |
| Example 22.27 | 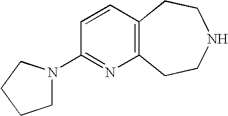 | 2-pyrrolidin-1-yl- 6,7,8,9-tetrahydro- 5H-pyrido[2,3- d]azepine | A | 42 mg, 65% |
| NMR | ¹H NMR (300 MHz, CDCl₃): δ(ppm) 7.16 (d, 1 H), 6.10 (d, 1 H), 3.43 (m, 6 H), 2.50-3.30 (m, 7 H), | | | |
| | 1.97 (m, 4 H). | | | |
| Example 22.28 | 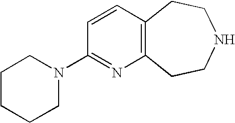 | 2-piperidin-1-yl- 6,7,8,9-tetrahydro- 5H-pyrido[2,3- d]azepine | A | 13 mg, 19% |
| NMR | ¹H NMR (300 MHz, CDCl₃): δ(ppm) 7.21 (d, 1 H), 6.45 (d, 1 H), 3.50 (m, 4 H), 3.27 (m, 7 H), | | | |
| | 3.00 (m, 2 H), 1.64 (br, 6 H). | | | |
| Example 22.29 | 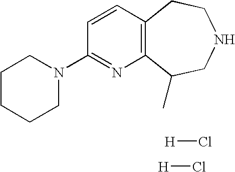 | 9-methyl-2-piperidin- 1-yl-6,7,8,9- tetrahydro-5H- pyrido[2,3-d]azepine dihydrochloride | B | 61 mg, 96% |
| NMR | ¹H NMR (300 MHz, MeOD): δ(ppm) 7.83 (d, 1 H), 7.25 (d, 1 H), 3.78 (m, 5 H), | | | |
| | 3.50 (m, 3 H), 3.20 (m, 3 H), 1.79 (br, 6 H), 1.55 (d, 3 H). | | | |
| Example 22.30 | 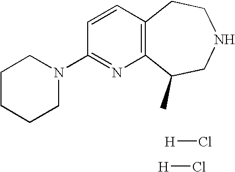 | (9R)-9-methyl-2- piperidin-1-yl-6,7,8,9- tetrahydro-5H- pyrido[2,3-d]azepine dihydrochloride | B | 42.5 mg |
| Example 22.31 | 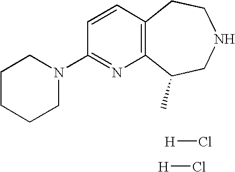 | (9S)-9-methyl-2- piperidin-1-yl-6,7,8,9- tetrahydro-5H- pyrido[2,3-d]azepine dihydrochloride | B | 42 mg |
| Example 22.32 | 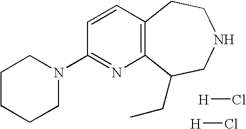 | 9-ethyl-2-piperidin-1- yl-6,7,8,9-tetrahydro- 5H-pyrido[2,3- d]azepine dihydrochloride | B | 20 mg, 68% |
| NMR | ¹H NMR (300 MHz, MeOD): δ(ppm) 7.86 (d, 1 H), 7.27 (d, 1 H), 3.75 (br, 4 H), | | | |
| | 3.00-3.70 (m, 4 H), 1.96 (m, 2 H), 1.79 (br, 6 H), 1.186 (t, 3 H), 1.07 (t, 3 H). | | | |
| Example 22.33 | 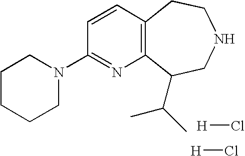 | 9-isopropyl-2- piperidin-1-yl-6,7,8,9- tetrahydro-5H- pyrido[2,3-d]azepine dihydrochloride | B | 36 mg, 52% |
| NMR | ¹H NMR (300 MHz, MeOD): δ(ppm) 7.87 (d, 1 H), 7.29 (d, 1 H), 3.76 (br, 6 H), | | | |
| | 2.90-3.65 (m, 5 H), 2.40 (m, H), 1.79 (br, 6 H), 1.18 (d, 3 H), 0.90 (d, 3 H). | | | |
| Example 22.34 | 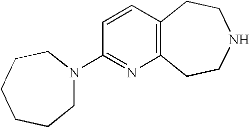 | 2-Azepan-1-yl- 6,7,8,9-tetrahydro- 5H- pyrido[2,3-d]azepine | A | 20 mg, 89% |
| NMR | ¹H NMR (300 MHz, CDCl₃): δ(ppm) 7.15 (d, 1 H), 6.24 (d, 1 H), 5.84 (br, 1 H), | | | |
| | 3.61 (t, 4 H), 3.12 (m, 6 H), 2.85 (m, 2 H), 1.76 (br, 4 H), 1.57 (m, 4 H). | | | |
| Example 22.35 | 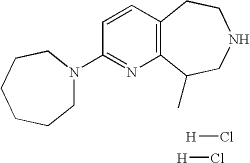 | 2-azepan-1-y-9- methyl-6,7,8,9- tetrahydro-5H- pyrido[2,3-d]azepine dihydrochloride | B | 49 mg, 74% |
| NMR | ¹H NMR (300 MHz, MeOD): δ(ppm) 7.82 (d, 1 H), 7.17 (d, 1 H), 3.94 (m, 1 H), | | | |
| | 3.79 (m, 4 H), 3.54 (m, 3 H), 3.00-3.40 (m, 3 H), 1.91 (br, 4 H), 1.67 (br, 4 H), 1.55 (d, 3 H). | | | |
| Example 22.36 | 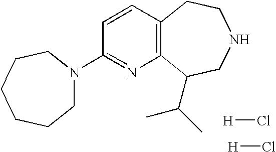 | 2-azepan-1-yl-9- isopropyl-6,7,8,9- tetrahydro-5H- pyrido[2,3-d]azepine dihydrochloride | B | 36 mg, 50% |
| NMR | ¹H NMR (300 MHz, MeOD): δ(ppm) 7.85 (d, 1 H), 7.20 (d, 1 H), 3.80 (m, 5 H), | | | |
| | 2.90-3.75 (m, 6 H), 2.40 (m, 1 H), 1.91 (br, 4 H), 1.66 (br, 4 H), 1.18 (d, 3 H), 0.90 (d, 3 H). | | | |
| Example 22.37 | 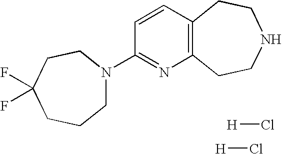 | 2-(4,4- difluoroazepan-1-yl)- 6,7,8,9-tetrahydro- 5H-pyrido[2,3- d]azepine dihydrochloride | B | 11.3 mg, 80% |
| NMR | ¹H NMR (300 MHz, MeOD): δ(ppm) 7.91 (d, 1 H), 7.21 (d, 1 H), 3.98 (m, 4 H), 3.10-3.60 (m, 8 H), | | | |
| | 2.20 (m, 6 H). | | | |
| Example 22.38 | 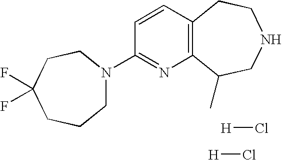 | 2-(4,4- difluoroazepan-1-yl)- 9-methyl-6,7,8,9- tetrahydro-5H- pyrido[2,3-d]azepine dihydrochloride | B | 11.4 mg, 80% |
| NMR | ¹H NMR (300 MHz, MeOD): δ(ppm) 7.87 (d, 1 H), 7.21 (d, 1 H), 3.88 (m, 4 H), | | | |
| | 3.54 (m, 3 H), 3.19 (m, 4 H), 2.25 (m, 6 H), 2.56 (d, 3 H). | | | |
| Example 22.39 | 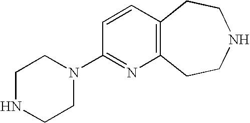 | 2-piperazin-1-yl- 6,7,8,9-tetrahydro- 5H-pyrido[2,3- d]azepine | A | 9.9 mg, 15% |
| NMR | ¹H NMR (300 MHz, DMSO-d6): δ(ppm) 7.45 (d, 1 H), 6.72 (d, 1 H), 3.65 (m, 4 H), 3.15 (m, 10 H), | | | |
| | 2.95 (m, 2 H). | | | |
| Example 22.40 | 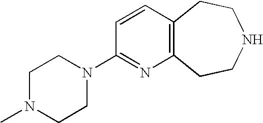 | 2-(4-methylpiperazin- 1-yl)-6,7,8,9- tetrahydro-5H- pyrido[2,3-d]azepine | A | 14 mg, 90% |
| NMR | ¹H NMR (300 MHz, DMSO-d6): δ(ppm) 7.21 (d, 1 H), 6.38 (d, 1 H), 3.50 (m, | | | |
| | 4 H), 2.98 (m, 6 H), 2.86 (m, 2 H), 2.62 (br, 1 H), 2.52 (m, 4 H), 2.35 (s, 3 H). | | | |
| Example 22.41 | 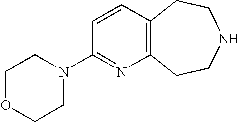 | 2-morpholin-4-yl- 6,7,8,9-tetrahydro- 5H-pyrido[2,3- d]azepine | A | 57 mg 100% |
| NMR | ¹H NMR (300 MHz, DMSO-d6): δ(ppm) 7.57 (d, 1 H), 6.43 (d, 1 H), 3.83 (m, | | | |
| | 4 H), 3.47 (m, 4 H), 3.15 (m, 6 H), 2.95 (m, 2 H). | | | |
| Example 22.42 | 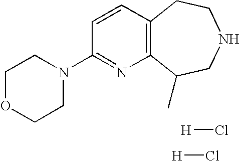 | 9-methyl-2- morpholin-4-yl- 6,7,8,9-tetrahydro- 5H-pyrido[2,3- d]azepine dihydrochloride | B | 59 mg 93% |
| NMR | ¹H NMR (300 MHz, MeOD): δ(ppm) 7.90 (d, 1 H), 7.25 (d, 1 H), 3.87 (m, 4 H), | | | |
| | 3.74 (m, 4 H), 3.53 (m, 3 H), 3.30 (m, 4 H), 1.56 (d, 3 H). | | | |
| Example 22.43 | 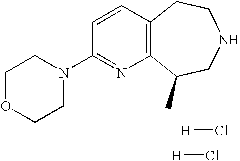 | (9R)-9-methyl-2- morpholin-4-yl- 6,7,8,9-tetrahydro- 5H-pyrido[2,3- d]azepine dihydrochloride | B | 47.5 mg 93% |
| Example 22.44 | 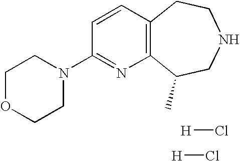 | (9S)-9-methyl-2- morpholin-4-yl- 6,7,8,9-tetrahydro- 5H-pyrido[2,3- d]azepine dihydrochloride | B | 48.2 mg 93% |
| Example 22.45 | 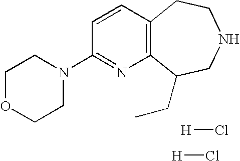 | 9-ethyl-2-morpholin- 4-yl-6,7,8,9- tetrahydro-5H- pyrido[2,3-d]azepine dihydrochloride | B | 55 mg 82% |
| NMR | ¹H NMR (300 MHz, MeOD): δ(ppm) 7.93 (d, 1 H), 7.27 (d, 1 H), 3.87 (m, 4 H), | | | |
| | 3.05-3.80 (m, 11 H), 1.96 (m, 2 H), 1.08 (t, 3 H). | | | |
| Example 22.46 | 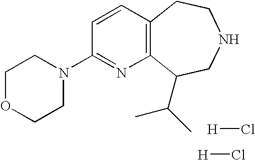 | 9-isopropyl-2- morpholin-4-yl- 6,7,8,9-tetrahydro- 5H-pyrido[2,3- d]azepine dihydrochloride | B | 64 mg 91% |
| NMR | ¹H NMR (300 MHz, MeOD): δ(ppm) 7.94 (d, 1 H), 7.29 (d, 1 H), 3.87 (m, 4 H), | | | |
| | 3.05-3.80 (m, 11 H), 2.41 (m, 1 H), 1.19 (d, 3 H), 0.91 (t, 3 H). | | | |
| Example 22.47 | 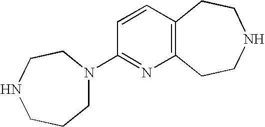 | 2-[1,4]Diazepan-1-yl- 6,7,8,9-tetrahydro- 5H-pyrido[2,3- d]azepine | A | 15 mg, 74% |
| NMR | ¹H NMR (300 MHz, CDCl₃): δ(ppm) 7.30 (d, 1 H), 6.45 (d, 1 H), 3.72 (t, 2 H), 3.33 (t, 2 H), 2.90- | | | |
| | 3.15 (m, 14 H). | | | |
| Example 22.48 | 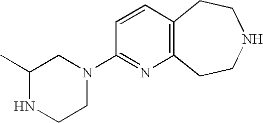 | 2-(3-Methyl- piperazin-1-yl)- 6,7,8,9- tetrahydro-5H- pyrido[2,3-d]azepine | A | 6 mg, 100% |
| NMR | ¹H NMR (300 MHz, MeOD): δ(ppm) 7.33 (d, 1 H), 6.57 (d, 1 H), 4.13 (m, 2 H), | | | |
| | 3.02 (m, 8 H), 2.86 (m, 4 H), 2.44 (m, 1 H), 1.17 (d, 3 H). | | | |
| Example 22.49 | 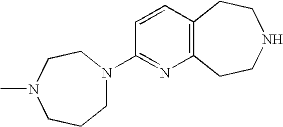 | 2-(4-Methyl- [1,4]diazepan-1-yl)- 6,7,8,9-tetrahydro- 5H-pyrido[2,3- d]azepine | A | 19 mg, 100% |
| NMR | ¹H NMR (300 MHz, MeOD): δ(ppm) 7.32 (d, 1 H), 6.43 (d, 1 H), 3.86 (m, 2 H), | | | |
| | 3.62 (t, 2 H), 3.19 (m, 6 H), 2.96 (m, 2 H), 2.83 (m, 2 H), 2.71 (m, 2 H), 2.46 (s, 3 H), 2.03 (m, 2 H). | | | |
| Example 22.50 | 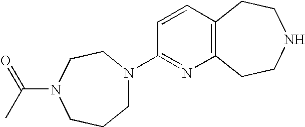 | 1-[4-(6,7,8,9- Tetrahydro-5H- pyrido[2,3-d]azepin- 2-yl)-[1,4]diazepan- 1-yl]-ethanone | A | 21 mg, 85% |
| NMR | ¹H NMR (300 MHz, MeOD): δ(ppm) 7.29 (d, 1 H), 6.46 (m, 1 H), 3.92 (m, | | | |
| | 2 H), 3.68 (m, 6 H), 3.47 (m, 2 H), 3.11 (m, 5 H), 2.92 (m, 2 H), 1.95 (br m, 5 H). | | | |
| Example 22.51 | 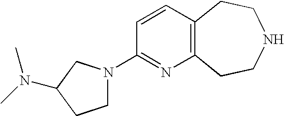 | Dimethyl-[1-(6,7,8,9- tetrahydro-5H- pyrido[2,3-d]azepin- 2-yl)-pyrrolidin-3-y]- amine | A | 19.5 mg, 100% |
| NMR | ¹H NMR (300 MHz, MeOD): δ(ppm) 7.32 (d, 1 H), 6.28 (d, 1 H), 3.78 (m, 1 H), | | | |
| | 3.64 (t, 1 H), 3.32 (m ,1 H), 3.19 (m, 8 H), 2.96 (m, 2 H), 2.35 (s, 6 H), 2.31 (m, 2 H). | | | |
| Example 22.52 | 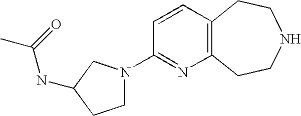 | N-[1-(6,7,8,9- Tetrahydro-5H- pyrido[2,3-d]azepin- 2-yl)-pyrrolidin-3-yl]- acetamide | | 12 mg, 43% |
| NMR | ¹H NMR (300 MHz, MeOD): δ(ppm) 7.25 (d, 1 H), 6.22 (d, 1 H), 4.44 (m, 1 H), | | | |
| | 3.67 (m, 1 H), 3.51 (m, 2 H), 3.34 (m, 1 H), 2.92 (br m, 8 H), 2.25 (m, 1 H), 1.97 (m, 4 H). | | | |
| Example 22.53 | 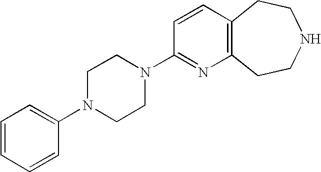 | 2-(4-phenylpiperazin- 1-yl)-6,7,8,9- tetrahydro-5H- pyrido[2,3-d]azepine | A | 13.4 mg, 59% |
| NMR | ¹H NMR (300 MHz, CDCl₃): δ(ppm) 7.30 (m, 3 H), 7.01 (d, 2 H), 6.90 (t, 1 H), | | | |
| | 6.45 (d, 1 H), 3.65 (m, 4 H), 3.30 (m, 4 H), 3.00 (m, 6 H), 2.81 (m, 2 H), 2.39 (br, 1 H). | | | |
| Example 22.54 | 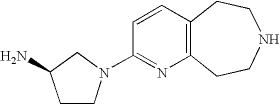 | (3R)-1-(6,7,8,9- tetrahydro-5H- pyrido[2,3-d]azepin- 2-yl)pyrrolidin-3- amine | A | 22.9 mg, 100% |
| NMR | ¹H NMR (300 MHz, MeOD): δ(ppm) 7.55 (d, 1 H), 6.57 (d, 1 H), 4.06 (m, | | | |
| | 1 H), 3.83 (m, 1 H), 3.66 (m, 3 H), 3.34 (m, 6 H), 3.08 (m, 2 H), 2.50 (m, 1 H), 2.22 (m, 1 H). | | | |
| Example 22.55 | 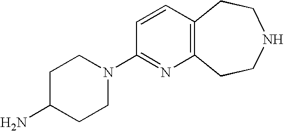 | 1-(6,7,8,9-tetrahydro- 5H-pyrido[2,3- d]azepin-2- yl)piperidin-4-amine | A | 20.3 mg, 100% |
| NMR | ¹H NMR (300 MHz, MeOD): δ(ppm) 7.30 (d, 1 H), 6.56 (d, 1 H), 4.27 (m, 2 H), | | | |
| | 2.90 (m, 11 H), 1.93 (m, 2 H), 1.44 (m, 2 H). | | | |
| Example 22.56 | 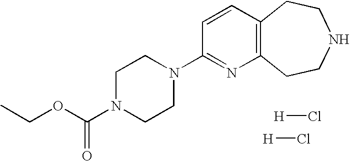 | ethyl 4-(6,7,8,9- tetrahydro-5H- pyrido[2,3-d]azepin- 2-yl)piperazine-1- carboxylate dihydrochloride | B | 12 mg, 78% |
| NMR | ¹H NMR (300 MHz, MeOD): δ(ppm) 7.93 (d, 1 H), 7.23 (d, 1 H), 4.19 (q, 2 H), | | | |
| | 3.75 (m, 8 H), 3.49 (m, 4 H), 3.36 (m, 2 H), 3.18 (m, 2 H), 1.30 (t, 3 H). | | | |
| Example 22.57 | 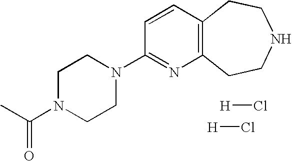 | 2-(4-acetylpiperazin- 1-yl)-6,7,8,9- tetrahydro-5H- pyrido[2,3-d]azepine dihydrochloride | B | 15.5 mg, 93% |
| NMR | ¹H NMR (300 MHz, MeOD): δ(ppm) 7.97 (d, 1 H), 7.27 (d, 1 H), 3.8 (m, 8 H), | | | |
| | 3.51 (m, 2 H), 3.48 (m, 2 H), 3.37 (m, 2 H), 3.20 (m, 2 H), 2.19 (s, 3 H). | | | |
| Example 22.58 | 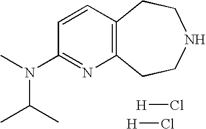 | N-isopropyl-N- methyl-6,7,8,9- tetrahydro-5H- pyrido[2,3-d]azepin- 2-amine dihydrochloride | B | 15.7 mg, 51% |
| NMR | ¹H NMR (300 MHz, MeOD): δ(ppm) 7.87 (d, 1 H), 7.21 (d, 1 H), 4.49 (m, 1 H), | | | |
| | 3.57 (m, 2 H), 3.47 (m, 2 H), 3.36 (m, 2 H), 3.18 (m, 2 H), 3.14 (s, 3 H), 1.34 (d, 6 H). | | | |
| Example 22.59 | 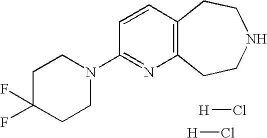 | 2-(4,4- difluoropiperidin-1- yl)-6,7,8,9- tetrahydro-5H- pyrido[2,3-d]azepine dihydrochloride | B | 17.9 mg, 55% |
| NMR | ¹H NMR (300 MHz, MeOD): δ(ppm) 7.97 (d, 1 H), 7.33 (d, 1 H), 3.91 (m, 4 H), | | | |
| | 3.52 (m, 4 H), 3.38 (m, 2 H), 3.20 (m, 2 H), 2.22 (m, 4 H). | | | |
| Example 22.60 | 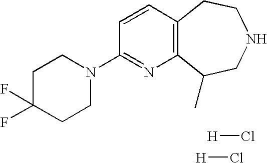 | 2-(4,4- difluoropiperidin-1- yl)-9-methyl-6,7,8,9- tetrahydro-5H- pyrido[2,3-d]azepine dihydrochloride | B | 21.4 mg, 90% |
| NMR | ¹H NMR (300 MHz, MeOD): δ(ppm) 7.93 (d, 1 H), 7.32 (d, 1 H), 3.90 (m, 5 H), | | | |
| | 3.56 (m, 3 H), 3.35 (m, 1 H), 3.19 (m, 2 H), 2.24 (m, 4 H), 1.57 (d, 3 H). | | | |
| Example 22.61 | 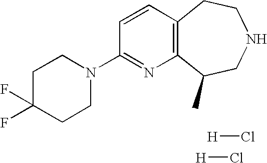 | (94)-2-(4,4- difluoropiperidin-1- yl)-9-methyl-6,7,8,9- tetrahydro-5H- pyrido[2,3-d]azepine dihydrochloride | B | 6.6 mg |
| Example 22.62 | 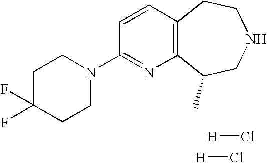 | (9S)-2-(4,4- difluoropiperidin-1- yl)-9-methyl-6,7,8,9- tetrahydro-5H- pyrido[2,3-d]azepine dihydrochloride | B | 7.5 mg |
| Example 22.63 | 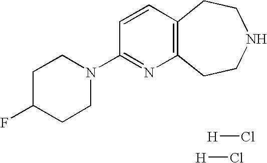 | 2-(4-fluoropiperidin- 1-yl)-6,7,8,9- tetrahydro-5H- pyrido[2,3-d]azepine dihydrochloride | B | 22.6 mg, 100% |
| NMR | ¹H NMR (300 MHz, MeOD): δ(ppm) 7.92 (d, 1 H), 7.29 (d, 1 H), 5.06, 4.91 (d, | | | |
| | m, 1 H), 3.83 (m, 4 H), 3.52 (m, 4 H), 3.37 (m, 2 H), 3.18 (m, 2 H), 2.08 (m, 4 H). | | | |
| Example 22.64 | 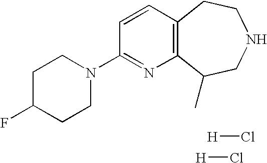 | 2-(4-fluoropiperidin- 1-yl)-9-methyl- 6,7,8,9-tetrahydro- 5H-pyrido[2,3- d]azepine dihydrochloride | B | 20.2 mg, 100% |
| NMR | ¹H NMR (300 MHz, MeOD): δ(ppm) 7.89 (d, 1 H), 7.30 (d, 1 H), 5.06, 4.92 (d | | | |
| | m, 1 H), 3.86 (m, 5 H), 3.58 (m, 3 H), 3.25 (m, 3 H), 2.08 (m, 4 H), 1.56 (d, 3 H). | | | |
| Example 22.65 | 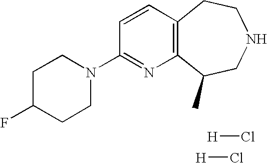 | (9R)-2-(4- fluoropiperidin-1-yl)- 9-methyl-6,7,8,9- tetrahydro-5H- pyrido[2,3-d]azepine dihydrochloride | B | 11.1 mg |
| Example 22.66 | 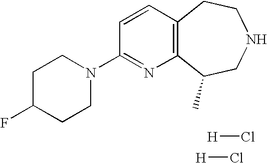 | (9S)-2-(4- fluoropiperidin-1-yl)- 9-methyl-6,7,8,9- tetrahydro-5H- pyrido[2,3-d]azepine dihydrochloride | B | 8.7 mg |
| Example 22.67 | 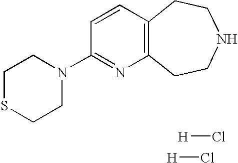 | 2-thiomorpholin-4-yl- 6,7,8,9-tetrahydro- 5H-pyrido[2,3- d]azepine dihydrochloride | B | 25 mg, 84% |
| NMR | ¹H NMR (300 MHz, MeOD): δ(ppm) 7.93 (d, 1 H), 7.27 (d, 1 H), 4.09 (m, 4 H), | | | |
| | 3.56 (m, 2 H), 3.51 (m, 2 H), 3.37 (m, 2 H), 3.19 (m, 2 H), 2.83 (m, 4 H). | | | |
| Example 22.68 | 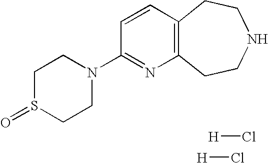 | 2-(1- oxidothiomorpholin- 4-yl)-6,7,8,9- tetrahydro-5H- pyrido[2,3-d]azepine dihydrochloride | B | 103 mg, 95% |
| NMR | ¹H NMR (300 MHz, MeOD): δ(ppm) 7.92 (d, 1 H), 7.25 (d, 1 H), 4.08 (m, 4 H), 3.20-3.60 (m, 8 H), | | | |
| | 2.85 (m, 4 H). | | | |
| Example 22.69 | 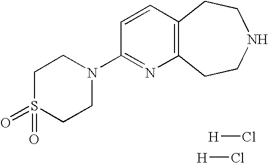 | 2-(1,1- dioxidothiomorpholin- 4-yl)-6,7,8,9- tetrahydro-5H- pyrido[2,3-d]azepine dihydrochloride | B | 6 mg, 65% |
| NMR | ¹H NMR (300 MHz, MeOD): δ(ppm) 7.64 (d, 1 H), 6.99 (d, 1 H), 4.85 (m, 4 H), 4.19 (m, 4 H), 3.32 (m, | | | |
| | 2 H), 3.11 (m, 6 H). | | | |
| Example 22.70 | 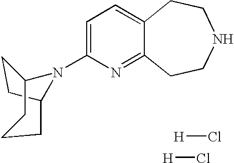 | 2-(8- azabicyclo[3.2.1]oct- 8-yl)-6,7,8,9- tetrahydro-5H- pyrido[2,3-d]azepine dihydrochloride | B | 6 mg, 67% |
| NMR | ¹H NMR (300 MHz, MeOD): δ(ppm) 7.83 (d, 1 H), 7.13 (d, 1 H), 4.73 (m, 2 H), 3.50 (m, 4 H), 3.14 (m, | | | |
| | 2 H), 1.60-2.25 (m, 12 H). | | | |
| Example 22.71 | 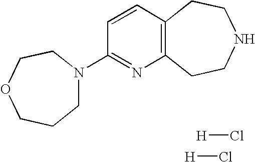 | 2-(1,4-oxazepan-4- yl)-6,7,8,9- tetrahydro-5H- pyrido[2,3-d]azepine dihydrochloride | B | 15 mg, 60% |
| NMR | ¹H NMR (300 MHz, MeOD): δ(ppm) 7.90 (d, 1 H), 7.22 (d, 1 H), 3.94 (m, 6 H), | | | |
| | 3.81 (m, 2 H), 3.58 (m, 2 H), 3.49 (m, 2 H), 3.37 (m, 2 H), 3.18 (m, 2 H), 2.05 (m, 2 H). | | | |
| Example 22.72 | 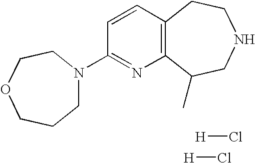 | 9-methyl-2-(1,4- oxazepan-4-yl)- 6,7,8,9-tetrahydro- 5H-pyrido[2,3- d]azepine dihydrochloride | B | 20.0 mg, 98% |
| NMR | ¹H NMR (300 MHz, MeOD): δ(ppm) 7.85 (d, 1 H), 7.21 (d, 1 H), 3.93 (m, 7 H), | | | |
| | 3.82 (m, 2 H), 3.52 (m, 3 H), 3.23 (m, H), 2.04 (m, 2 H), 1.55 (d, 3 H). | | | |
| Example 22.73 | 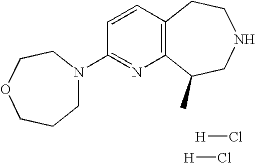 | (9R)-9-methyl-2-(1,4- oxazepan-4-yl)- 6,7,8,9-tetrahydro- 5H-pyrido[2,3- d]azepine dihydrochloride | B | 10 mg |
| Example 22.74 | 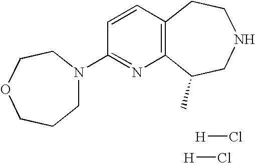 | (9S)-9-methyl-2-(1,4- oxazepan-4-yl)- 6,7,8,9-tetrahydro- 5H-pyrido[2,3- d]azepine dihydrochloride | B | 10 mg |
| Example 22.75 | 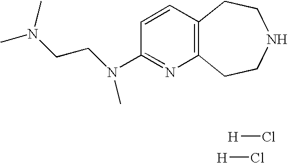 | N,N,N′-trimethyl-N′- (6,7,8,9-tetrahydro- 5H-pyrido[2,3- d]azepin-2-yl)ethane- 1,2-diamine dihydrochloride | B | 38.1 mg, 100% |
| NMR | ¹H NMR (300 MHz, MeOD): δ(ppm) 7.97 (d, 1 H), 7.31 (d, 1 H), 4.20 (m, 2 H), | | | |
| | 3.71 (m, 2 H), 3.53 (m, 4 H), 3.40 (m, 5 H), 3.23 (m, 2 H), 3.00 (s, 6 H). | | | |
| Example 22.76 | 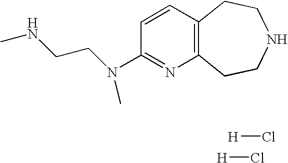 | N,N′-dimethyl-N- (6,7,8,9-tetrahydro- 5H-pyrido[2,3- d]azepin-2-yl)ethane- 1,2-diamine dihydrochloride | B | 38.7 mg, 100% |
| NMR | ¹H NMR (300 MHz, MeOD): δ(ppm) 7.96 (d, 1 H), 7.28 (d, 1 H), 4.12 (m, 2 H), | | | |
| | 3.70 (m, 2 H), 3.50 (m, 2 H), 3.38 (m, 7 H), 3.22 (m, 2 H), 2.79 (s, 3 H). | | | |
| Example 22.77 | 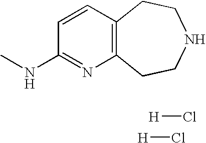 | N-methyl-6,7,8,9- tetrahydro-5H- pyrido[2,3-d]azepin- 2-amine dihydrochloride | B | 22.7 mg, 71% |
| NMR | ¹H NMR (300 MHz, MeOD): δ(ppm) 7.81 (d, 1 H), 6.91 (d, 1 H), 3.00-3.60 (m, 11 H). | | | |
| Example 22.78 | 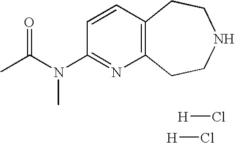 | N-methyl-N-(6,7,8,9- tetrahydro-5H- pyrido[2,3-d]azepin- 2-yl)acetamide dihydrochloride | B | 60 mg, 98% |
| NMR | ¹H NMR (300 MHz, MeOD): δ(ppm) 8.24 (d, 1 H), 7.64 (d, 1 H), 3.30-3.60 (m, 11 H), 2.27 (m, 3 H) | | | |
| Example 22.79 | 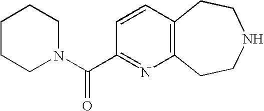 | 2-(piperidin-1- ylcarbonyl)-6,7,8,9- tetrahydro-5H- pyrido[2,3-d]azepine | B | 104.9 mg, 100% |
| NMR | ¹H NMR (300 MHz, MeOD): δ(ppm) 7.44 (d, 1 H), 7.28 (d, 1 H), 3.69 (m, 2 H), | | | |
| | 3.42 (m, 2 H), 3.16 (m, 2 H), 2.90 (m, 6 H), 2.35 (br, 1 H), 1.65 (br, s, 4 H), 1.53 (br s, 2 H). | | | |
| Example 22.80 | 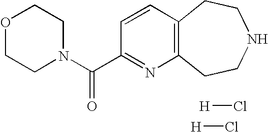 | 2-(morpholin-4- ylcarbonyl)-6,7,8,9- tetrahydro-5H- pyrido[2,3-d]azepine dihydrochloride | B | 77.8 mg, 89% |
| NMR | ¹H NMR (300 MHz, CDCl₃ + MeOD): δ(ppm) 8.48 (d, 1 H), 7.99 (d, 1 H), 3.40-3.80 (m, 16 H). | | | |
| Example 22.81 | 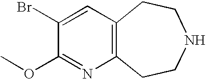 | 3-bromo-2-methoxy- 6,7,8,9-tetrahydro- 5H-pyrido[2,3- d]azepine | A | 25 mg, 77% |
| NMR | ¹H NMR (300 MHz, MeOD): δ(ppm) 7.52 (s, 1 H), 3.97 (s, 3 H), 3.80 (br, 1 H), 3.02 (m, 6 H), | | | |
| | 2.82 (m, 2 H). | | | |
| Example 22.82 | 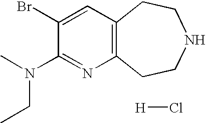 | 3-bromo-N-ethyl-N- methyl-6,7,8,9- tetrahydro-5H- pyrido[2,3-d]azepin- 2-amine hydrochloride | B | 45 mg, 86% |
| NMR | ¹H NMR (300 MHz, DMSO-d6): δ(ppm) 9.23 (br, 1 H), 7.78 (s, 1 H), 3.22 (m, | | | |
| | 8 H), 3.00 (m, 2 H), 2.81 (m, 3 H), 1.12 (t, 3 H). | | | |
| Example 22.83 | 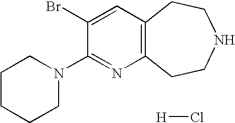 | 3-bromo-2-piperidin- 1-yl-6,7,8,9- tetrahydro-5H- pyrido[2,3-d]azepine hydrochloride | B | 61 mg, 96% |
| NMR | ¹H NMR (300 MHz, DMSO-d6): δ(ppm) 9.32 (br, 1 H), 7.81 (s, 1 H), 3.13 (m, 10 H), 3.02 (m, 2 H), | | | |
| | 1.56 (m, 6 H). | | | |
| Example 22.84 | 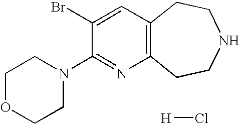 | 3-bromo-2- morpholin-4-yl- 6,7,8,9-tetrahydro- 5H-pyrido[2,3- d]azepine hydrochloride | B | 59 mg, 93% |
| NMR | ¹H NMR (300 MHz, DMSO-d6): δ(ppm) 9.44 (br, 1 H), 7.85 (s, 1 H), 3.92 (m, 4 H), 3.17 (m, 10 H), | | | |
| | 3.05 (m, 2 H). | | | |
| Example 22.85 | 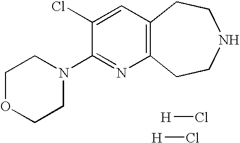 | 3-chloro-2- morpholin-4-yl- 6,7,8,9-tetrahydro- 5H-pyrido[2,3- d]azepine dihydrochloride | B | 21 mg, 55% |
| NMR | ¹H NMR (300 MHz, DMSO-d6): δ(ppm) 8.00 (s, 1 H), 3.88 (m, 4 H), 3.35 (m, 10 H), 3.20 (m, 2 H). | | | |
| Example 22.86 | 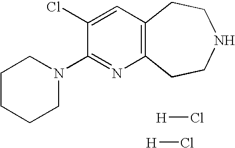 | 3-chloro-2-piperidin- 1-yl-6,7,8,9- tetrahydro-5H- pyrido[2,3-d]azepine dihydrochloride | B | 34 mg, 72% |
| NMR | ¹H NMR (300 MHz, MeOD): δ(ppm) 8.10 (s, 1 H), 3.68 (m, 4 H), 3.42 (m, 6 H), | | | |
| | 3.26 (m, 2 H), 1.95 (m, 4 H), 1.79 (m, 2 H). | | | |
| Example 22.87 | 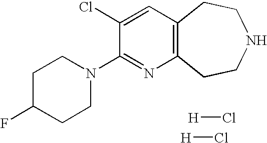 | 3-chloro-2-(4- fluoropiperidin-1-yl)- 6,7,8,9-tetrahydro- 5H-pyrido[2,3- d]azepine dihydrochloride | B | 15 mg, 64% |
| NMR | ¹H NMR (300 MHz, MeOD): δ(ppm) 7.90 (s, 1 H), 4.98, 4.81 (dm, 1 H), 3.60 | | | |
| | (m, 2 H), 3.35 (m, 8 H), 3.16 (m, 2 H), 2.04 (m, 4 H). | | | |
| Example 22.88 | 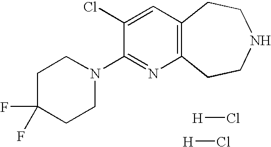 | 3-chloro-2-(4,4- difluoropiperidin-1- yl)-6,7,8,9- tetrahydro-5H- pyrido[2,3-d]azepine dihydrochloride | B | 5 mg, 35% |
| NMR | ¹H NMR (300 MHz, MeOD): δ(ppm) 7.64 (s, 1 H), 3.45 (m, 4 H), 3.31 (m, 6 H), 3.08 (m, 2 H), | | | |
| | 2.10 (m, 4 H). | | | |
| Example 22.89 | 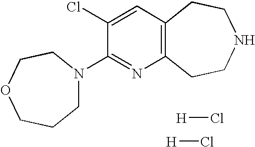 | 3-chloro-2-(1,4- oxazepan-4-yl)- 6,7,8,9-tetrahydro- 5H-pyrido[2,3- d]azepine dihydrochloride | B | 12 mg, 72% |

| | |
|---|---|
| NMR | ¹H NMR (300 MHz, MeOD): δ(ppm) 7.72 (s, 1 H), 3.82 (m, 12 H), 3.37 (m, 2 H), 3.08 (m, 2 H), |
| | 2.07 (m, 2 H). |

### Example 22.90

### 2-Isopropyl-6,7,8,9-tetrahydro-5H-pyrido[2,3-d]azepine

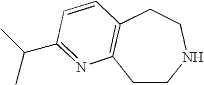

2-Isopropenyl-6,7,8,9-tetrahydro-5H-pyrido[2,3-d]azepine (8.5 mg) was treated with palladium hydroxide and methanol under hydrogen (1 atm) for 3 hours. The reaction mixture was concentrated, diluted with dichloromethane, filtered and concentrated again to give the product. The product was treated with hydrochloric acid in diethyl ether to give the hydrochloric acid salt (2 salt equivalents, 9.2 mg). ¹H NMR (300 MHz, CDCl₃): δ (ppm): free base 7.33 (d, 1H), 6.93 (d, 1H), 3.18 (m, 2H), 3.02 (m, 4H), 2.90 (m, 2H), 1.67 (br, 1H), 1.29 (s, 3H).

In a similar manner the following compound was synthesized:

| Example | Structure | Name | Yield |
|---|---|---|---|
| Example 22.91 | 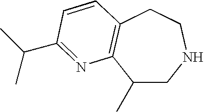 | 2-isopropyl-9- methyl-6,7,8,9- tetrahydro-5H- pyrido[2,3- d]azepine | 11 mg, 48.5% |

| | |
|---|---|
| NMR | ¹H NMR (300 MHz, CDCl₃): δ (ppm): 7.30 (d, 1H), 6.89 (d, |
| | 1H), 3.26 (m, 1H), 2.90 (m, 7H), 2.38 (br, 1H), 1.39 (d, 3H), |
| | 1.28 (d, 6H). |

### Evaluation of Biological Activity

Demonstration of the activity of the compounds of the present invention may be accomplished through in vitro, ex vivo and in vivo assays that are well known in the art, including the assays described in the following examples.

### Materials and Methods

Activation of the Gq coupled 5-HT₂ receptors stimulates phospholipase C activity and leads to formation of inositol trisphosphate (IP3) and the subsequent release of calcium from intracellular stores. Functional activity of Gq coupled receptors can be quantified in a FLIPR assay by measuring intracellular calcium levels with calcium sensitive dyes (using a fluorescence imaging plate reader, FLIPR) and in a Phosphatidyl Inositol Hydrolysis Assay (IP accumulation assay) which measures IPs derived from IP3. Both assays provide robust functional readouts of receptor activation.

Cell Culture Stable cell lines expressing human 5-HT2A, 5-HT2B and 5HT-2C (both INI and VSV isoforms) receptors were created in an MHEK cell background (an HEK293-based cell background which also expresses the Macrophage Scavenger Receptor 1, to increase the adherence of cells to tissue culture plates). Recombinant cell lines were cultured in Growth Medium (High glucose DMEM (Hyclone) with 10% dialyzed fetal bovine serum (Hyclone), and L-glutamine (Gibco; 0.8 mM for 5HT2A and 2C, 2.0 mM for 5HT2B), and grown under selection with 200 μg/ml Zeocin (Invitrogen), and either 200 μg/ml Hygromycin B (Invitrogen for 5HT2A and 5HT2C) or 500 ug/ml Geneticin (Invitrogen for 5HT2B).

FLIPR assay methodology: Cells that recombinantly expressed the 5HT2 receptors were enzymatically dissociated with Trypsin/EDTA 0.25% (Hyclone) 24 hours prior to testing, and seeded at 60,000 cells per well in 100 μl Growth Medium in black sided, clear bottom 96 well plates (Greiner, BioExpress) at 37° C. and 5% CO₂. On the day of the assay, Growth Medium was removed by aspiration, and 801 of Assay Buffer (20 mM HEPES, 146 mM Sodium Chloride, 5 mM Potassium Chloride, 1 mM Magnesium Chloride, 1 mg/mL BSA, 1 mg/mL Glucose, 1 mM CaCl₂, pH 7.4, supplied by Amresco), containing 6 μM Fluo-3 AM and 0.01% pluronic acid (Biotium Inc., Hawyard, Calif.) was added. Cells were incubated in the Fluo-3 solution for 60 minutes in the dark at room temperature. The Fluo-3 solution was then removed by aspiration and cells were washed twice with assay buffer leaving 160 μl in each well.

All compounds were prepared at 5 times their final concentration prior to the online addition (40 μl) in the FLIPR (MDS Inc., Sunnyvale, Calif.). The fluorescent intensity was measured at 1 second intervals for 10 seconds prior to the compound addition and 65 seconds after the compound addition. All responses were measured as the peak height of the fluorescent response over baseline, within the sample period. Non-linear regression of the relative fluorescence unit (RFU) change was used to determine agonist potency. Antagonist activity was measured after pre-incubation of cells with compound for 30 minutes at room temperature, followed by the online addition of agonist (5-HT, EC80) in the FLIPR. Antagonist activity was determined by normalizing the response to the maximal 5-HT response in the absence of test compound.

Phosphatidyl Inositol Hydrolysis Assay: 24 hours prior to testing, cells were plated in poly-D-Lysine-coated 96 well plates (VWR) at 100,000 cells/well in 200 μl culture medium containing 10 μCi/ml of [³H]-myo-lnositol (Perkin Elmer). Cell monolayers were washed twice with HBSS (HEPES Buffered Saline solution: 20 mM HEPES, 146 mM NaCl, 4.2 mM KCl, 0.5 mM MgCl₂, 0.1% Glucose, pH 7.4). The cell monolayers were pre-incubated for 5 minutes at 37° C. in 100 μl/well HBSS containing 10 mM LiCl. Compounds were tested for agonist activity in duplicate at concentrations ranging from 3 nM to 30 μM. Compounds were added (100 μl) at 2 times the required final concentration and incubated for 30 minutes at 37° C. Medium was aspirated and the soluble 3H-inositol phosphates were extracted from the cells by adding 100 μl/well of ice-cold 5% perchloroacetic acid solution. Plates were placed on ice for 1 hour, and extracts collected in a 2 ml, 96 well, polypropylene, round bottom Uniplate (VWR). Cell extracts were neutralized with 150-170 μl HEPES/KOH (0.375/0.75 M) containing a pH indicator until all solutions turned pale green. 600 μl HEPES/EDTA (2.5/0.5 mM, pH 7.4) was then added to all tubes, and contents were transferred to a 96 well PALL Filter Plate (VWR) loaded with 600 μl/well of Dowex resin (Dowex AG-1×8 formate form, 200-400 mesh, Bio-Rad, equilibrated in HEPES/EDTA (2.5/0.5 mM, pH 7.4). The Filter Plate was then placed in a vacuum manifold and a gentle vacuum was applied. Total phosphatidyl inositols were eluted with 800 μl 30 mM ammonium formate, and the eluate was discarded. Total inositol phosphates were eluted with 600 μl (2×300 μl) 700 mM ammonium formate/100 mM formic acid and collected in a clean 2 ml, 96 well, polypropylene, round bottom Uniplate. 75 μl eluate was transferred to a Hewlett Packard Optiplate and 150 μl Scint 40 was added to each well. The plate was sealed with a Topseal (Packard) and shaken for 1 minute on a platform plate shaker. Plates were counted in the Hewlett Packard Topcount to quantify the amount of radioactivity in each well.

Animals and housing: Male, Sprague-Dawley rats or CD-1 mice were used for all studies. All animals were allowed ad-lib access to food and water except during experiment. Animals were housed within an animal vivarium maintained under a 12 h light:dark cycle (lights on: 07:00 h), and all experiments were conducted in the animals' light phase. For all experiments, animals were habituated to the vivarium for a minimum of 72 h before experimentation. The experimental procedures used in the present investigation were conducted under the Association for Assessment and Accreditation of Laboratory Animal Care (AAALAC) and the Canadian Council on Animal Care (CCAC) guidelines.

Test Compounds All compounds were dissolved in 5% Tween 80 in saline and injected in a dose volume of 5 ml/kg or 10 ml/kg (rat), and 10 ml/kg (mouse). Compounds were administered by either the oral or intraperitoneal route.

Mouse hypolocomotion assay: Selective 5-HT2C receptor agonists have been reported to produce hypolocomotion in rodent species by a relatively well defined CNS mechanism. A mouse locomotor assay was therefore used to screen compounds. Male, CD-1 mice were administered test compound 15 min before placement in a chamber where locomotor activity was measured through photocell beam breaks. Test compounds were administered either by the oral or intraperitoneal route.

Deprivation-induced feeding in the rat: Male Sprague-Dawley rats (Charles River, St. Constant, Quebec, Canada) of approximate weight 180-200 g were pair housed on arrival in the animal facility (lights on 7:00-19:00 h). After a 7 day acclimitisation period where the animals received ad-libitum access to standard rodent lab chow (Harlan Teklad rodent maintenance diet, 2014; Harlan Teklad, Madison, Wis.), the animals were trained to receive a daily ration of lab chow in distinct chambers over a 2 h period. Animals were singly housed for the duration of this period and food intakes over the 2 h access period were measured by weighing food containers before and after feeding periods, with correction for spillage. After the daily 2 h food access period, the animals were returned to their holding cage with no further daily food allowance. Water was available ad-libitum. Body weights were recorded daily.

Once the daily food intake had stabilized (after one to two weeks training), a dose of a test compound (or vehicle as control) was administered 10 to 15 minutes before the beginning of the 2 hr. food access period, and food intake over that period was measured as during the training period.

Test compound or vehicle was administered on Tuesdays and Fridays, with drug free (washout) days in between. Typically the animals received 3 doses of test compound and vehicle in a counterbalanced sequence.

### Schedule-Induced Polydipsia

Food deprived rats exposed to intermittent, uncontrollable presentations of food will drink quantities of water that are far in excess of their normal daily intake and in excess of their intake when given food at one time (Falk J L (1961) Production of polydipsia in normal rats by an intermittent food schedule. Science 133: 195-196). This excessive behaviour is persistent and has been proposed as a model of obsessive-compulsive disorder based on pharmacological validation and symptomatic similarities (Woods, A. et al. (1993) Selective serotonin re-uptake inhibitors decrease schedule-induced polydipsia in rats: a potential model for obsessive compulsive disorder. Psychopharmacology 112: 195-198).

Male Sprague-Dawley rats (Charles River, St. Constant, Quebec, Canada) of approximate weight 180-200 g are pair housed on arrival in the animal facility (lights on 7:00-19:00 h). After a 7 day acclimitisation period where the animals receive ad-libitum access to standard rodent lab chow (Harlan Teklad rodent maintenance diet, 2014; Harlan Teklad, Madison, Wis.), the animals are trained to receive single 45 mg food pellets under a fixed time interval of 60 s over a 2 h period within an operant chamber equipped with a water bottle. Thus during the 2 h session, the rats can earn a maximum of 120 pellets. The total volume of water consumed by rats during this 2 h period is recorded. Daily food allowance is supplemented by a 45 min access period sometime between 15:00-18:00 h.

Once daily fluid intakes within the 2 h test session become stable over days (approximately ±15%), the rats may be dosed orally or parentally with vehicle or test compound. Test compound or vehicle is administered on Tuesdays and Fridays with drug free (washout) days in between. Typically the animals will receive 3 doses of test compound and vehicle in a counterbalanced sequence.

A modification to the above procedure is to pre-treat rats with either vehicle or a selective 5-HT_{2C} receptor antagonist, 6-chloro-5-methyl-N-(2-(2-methylpyridin-3-yl-oxy)pyridine-5-yl)aminocarbonyl)-2,3-dihydroindole (1 mg/kg in 8% HPCD, 25 mM citric acid in saline) prior to the oral or parental dose of test compound.

### s.c Pentylenetetrazol Assay

Antagonism of clonic-tonic seizures produced by chemical convulsants such as pentylenetetrazol have been widely utilized to identify novel anticonvulsants.

Male, CD-1 mice (Charles River, St. Constant, Quebec, Canada) of approximate body weight 20-30 g are housed in groups of four on arrival at the facility. Food (Harlan Teklad rodent maintenance diet, 2014; Harlan Teklad, Madison, Wis.) and water are available ad-libitum. After a minimum 3 day acclimatization period the animals would be tested in a s.c pentylenetetrazol assay—which is considered both a model of primary generalized convulsive seizures and non-convulsive absence (petit mal) seizures (Upton, N. (1994) Mechanisms of action of new antiepileptic drugs; rational design and serendipitous findings. Trends Pharmacol. Sci. 15: 456-463).

The experiment is conducted within a single day with animals receiving a single pretreatment, i.e independent groups design. Following drug or vehicle control treatment by either oral, or parenteral route, the animals would receive pentylenetetrazol (85 mg/kg mice) administered by the subcutaneous route. The dose of pentylenetetrazol is selected as it is of sufficient intensity to induce a clonic seizure in the majority of animals, i.e a CD97 dose. The animals are restrained by hand to deliver the chemical convulsant, following which the animals are released and transferred to a test cage to permit observation of the subsequent seizure throughout its course. The animal would receive a single pentylenetetrazol injection and would be terminated on reaching endpoint, i.e clonic seizure. If an animal displays no seizure activity after 60 min it is considered protected and the experiment completed as endpoint reached.

Approximately 10-20 min prior to the PTZ test, a parallel tests of motor function using the rotorod would be undertaken to establish a therapeutic index (TI), e.g ratio between the ED50 dose required to block seizures, compared to ED50 dose required to disrupt motor function in same species. The rotorod test consists of placing the animal on a rotating treadmill (a rod) traveling at a constant speed of 16 r.p.m. The dependant measure is the time that the animal remains on the rod before falling. Up to three separate measures may be taken to get a meaningful measure of performance.

A modification to the above procedure is to pretreat mice with either vehicle or a selective 5-HT_{2C} receptor antagonist, 6-chloro-5-methyl-N-(2-(2-methylpyridin-3-yl-oxy)pyridine-5-yl)aminocarbonyl)-2,3-dihydroindole (1 mg/kg in 8% HPCD, 25 mM citric acid in saline) prior to the oral or parental dose of test compound.

### Amphetamine-Induced Hyperlocomotion

Antagonism of increased locomotion produced by the psychostimulant amphetamine in rodents is a feature of many drugs with antipsychotic property in man. As such reversal of amphetamine hyperlocomotion is a widely used preclinical test to detect novel drugs for the treatment of schizophrenia.

Male Sprague-Dawley rats (Charles River, St. Constant, Quebec, Canada) of approximate weight 200 g are pair housed on arrival in the animal facility (lights on 7:00-19:00 h). After a 7 day acclimitisation period where the animals receive ad-libitum access to standard rodent lab chow (Harlan Teklad rodent maintenance diet, 2014; Harlan Teklad, Madison, Wis.) the animals may undergo behavioural testing.

Animals would be singly placed within the test apparatus (Perspex chamber of dimensions: rat 42 cm×42 cm×30 cm (L×W×H)) for a limited time period (approximately 30 min) to habituate to the novel environment. After such habituation period has passed, animals will be treated with test article or vehicle control via the oral, or parental route, and then returned to the observation test chambers. After a predetermined period, the animals would be dosed with either saline vehicle or d-amphetamine (0.5 mg/kg) by the intraperitoneal route and returned to the test chamber for 2 h. While in the test chamber, the animal's activity will be monitored automatically by infrared sensors and/or manually by an experimenter for expression of ‘normal’ behaviors such as sniffing, grooming, rearing, and ‘abnormal’ behaviors such as ‘circling’. At the completion of such test, the animals will be returned to their holding cages.

A modification to the above procedure is to pretreat rats with either vehicle or a selective 5-HT_{2C} receptor antagonist, 6-chloro-5-methyl-N-(2-(2-methylpyridin-3-yl-oxy)pyridine-5-yl)aminocarbonyl)-2,3-dihydroindole (1 mg/kg in 8% HPCD, 25 mM citric acid in saline) prior to the oral or parental dose of test compound.

### 5-HT_{2C} Agonist Activity

Table 1 shows the 5-HT_{2C} agonist potency of compounds in accordance with the invention, determined by FLIPR assay described above.

### Mouse Hypolocomotion

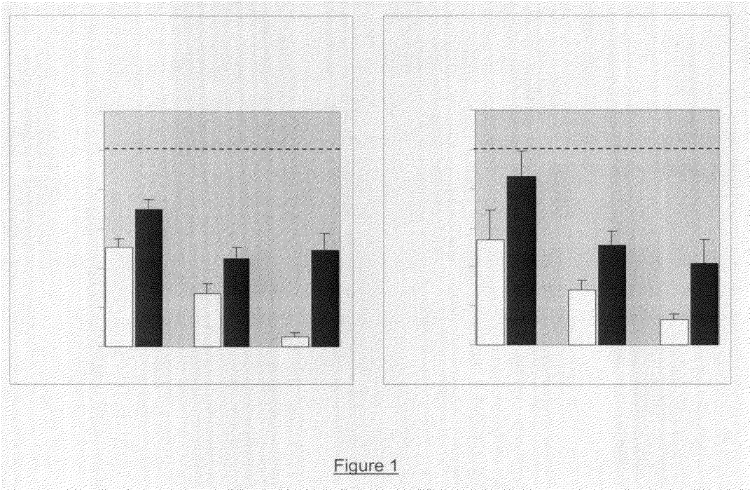

FIG. 1 shows the effect of two exemplary compounds of the invention on mouse locomotion after either oral or intraperitoneal injection.

Pre-treatment with the 5-HT_{2C} antagonist SB242084 blocked the effect of the test compounds (data not shown).

### Rat Deprivation Induced Feeding Assay

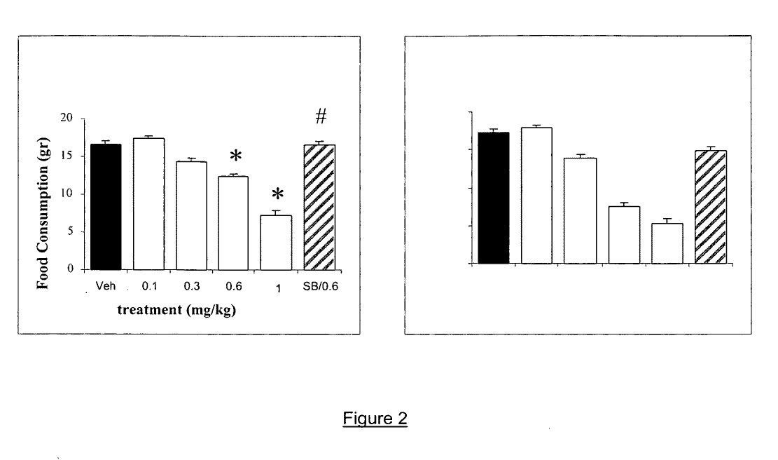

FIG. 2 shows the dose-related reduction in food intake in rats treated intraperitoneally with two exemplary compounds of the invention.

Pre-treatment of rats with the selective 5-HT_{2C} antagonist SB 242084 blocked the effect of the agonist compounds, as shown by the hatched bars.

The description as set forth is not intended to be exhaustive or to limit the scope of the invention. Many modifications and variations are possible in light of the above teaching without departing from the spirit and scope of the following claims. It is intended that the scope of the present invention be defined by the claims appended hereto, giving full cognizance to equivalents in all respects.

TABLE 1

| | FLIPR EC₅₀ μM h5-HT_{2c} |
|---|---|
| Structure | (vsv) |
| 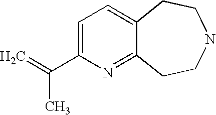 | 0.0791 |
| 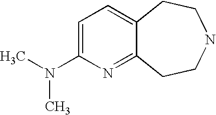 | 0.2133 |
| 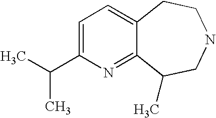 | 0.2951 |
| 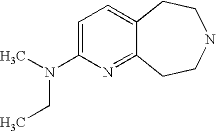 | 0.018 |
| 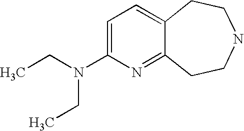 | 0.0773 |
| 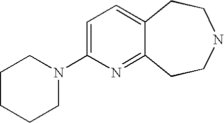 | 0.0106 |
| 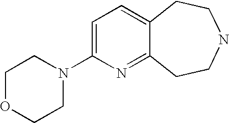 | 0.0639 |
| 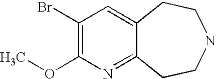 | 0.0641 |
| 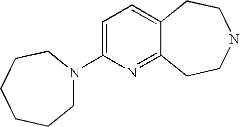 | 0.0102 |
| 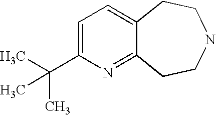 | 0.106 |
| 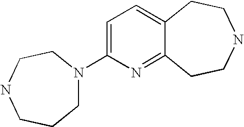 | 0.2617 |
| 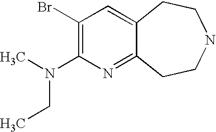 | 0.1003 |
| 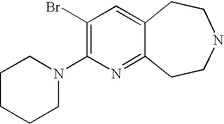 | 0.192 |
| 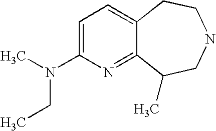 | 0.0292 |
| 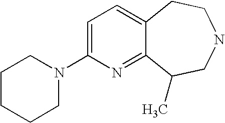 | 0.0131 |
| 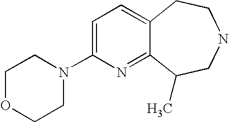 | 0.0477 |
| 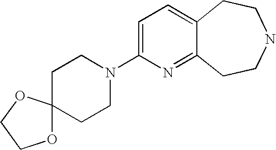 | 0.2902 |
| 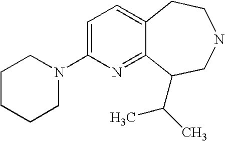 | 0.0654 |
| 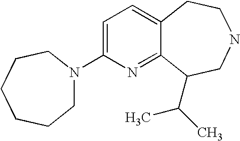 | 0.1394 |
| 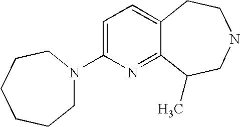 | 0.0062 |
| 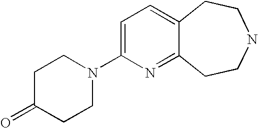 | 0.2989 |
| 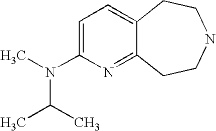 | 0.0854 |
| 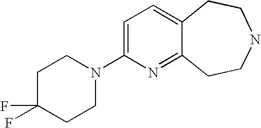 | 0.0114 |
| 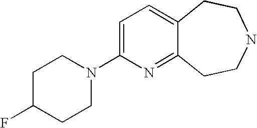 | 0.0079 |
| 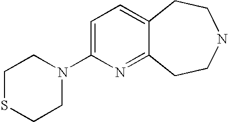 | 0.0023 |
| 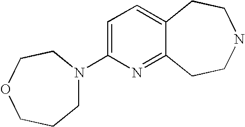 | 0.0105 |
| 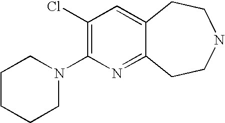 | 0.0742 |
| 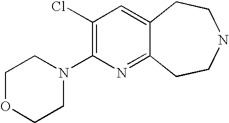 | 0.1758 |
| 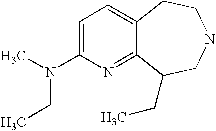 | 0.0359 |
| 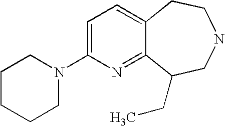 | 0.0164 |
| 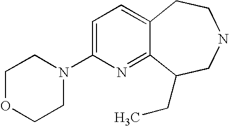 | 0.1005 |
| 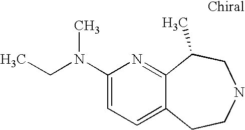 | 0.0083 |
| 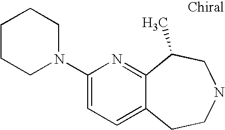 | 0.0021 |
| 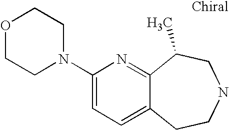 | 0.0581 |
| 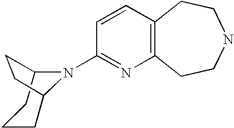 | 0.1378 |
| 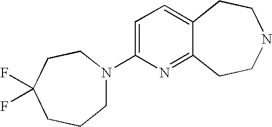 | 0.024 |
| 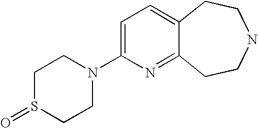 | 0.0051 |
| 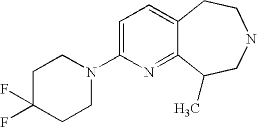 | 0.0294 |
| 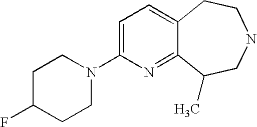 | 0.0136 |
| 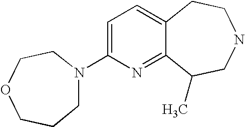 | 0.0217 |
| 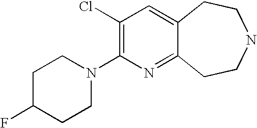 | 0.0958 |
| 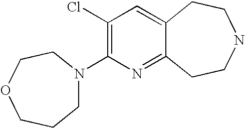 | 0.0678 |

We claim:

1. A compound of Formula I: 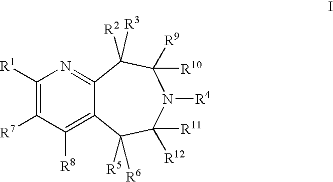
wherein:
R¹ to R³ and R⁵ to R¹² are independently selected from H, halo, hydroxy, cyano, nitro, alkyl, alkoxy, CH₂OH, haloalkyl, O-haloalkyl, hydroxyalkyl, cyanoalkyl, alkenyl, alkynyl, cycloalkyl, cycloalkenyl, heterocycloalkyl, heterocycloalkenyl, aryl, heteroaryl, alkylaryl, alkylheteroaryl, O-cycloalkyl, O-heterocycloalkyl, alkylene-O-alkyl, alkylene-O-cycloalkyl, alkylene-O-heterocycloalkyl, alkylene-O-alkylene-cycloalkyl, alkylene-O-alkylene-heterocycloalkyl, S-alkyl, S(O)-alkyl, S(O)₂-alkyl, S-cycloalkyl, S(O)-cycloalkyl, S(O)₂-cycloalkyl, S-heterocycloalkyl, S(O)-heterocycloalkyl, S(O)₂-heterocycloalkyl, O-aryl, O-heteroaryl, N(H)alkyl, N(alkyl)alkyl, N(H)-aryl, N(alkyl)-aryl, N(H)-heteroaryl, N(alkyl)-heteroaryl, alkylene-O-aryl, alkylene-O-heteroaryl, alkylene-O-alkylene-aryl, alkylene-O-alkylene-heteroaryl, S-aryl, S-heteroaryl, S(O)-aryl, S(O)-heteroaryl, S(O)₂-aryl, S(O)₂-heteroaryl, C(O)alkyl, OC(O)alkyl, C(O)Oalkyl, C(O)N(H)alkyl, C(O)N(alkyl)alkyl, S(O)₂N(H)alkyl or S(O)₂N(alkyl)alkyl; R² and R³, R⁵ and R⁶, R⁹ and R¹⁰, and/or R¹¹ and R¹², together with the carbon atom to which they are attached, form a cycloalkyl group; and
R⁴ is selected from H, alkyl, alkenyl, alkynyl, cycloalkyl, cycloalkenyl, alkylene-O-alkyl, alkylene-O-cycloalkyl, alkylene-O-alkylene-cycloalkyl;
and/or a pharmaceutically-acceptable salt, hydrate, solvate, isoform, tautomer, optical isomer, or combination thereof.

2. A compound according to claim 1, wherein any cyclic group is substituted with one or more R¹³, R¹³ being selected from F, Cl, Br, I, CN, nitro, hydroxy, oxo, C₁₋₆-alkyl, OC₁₋₆-alkyl, C₁₋₆-alkylhalo or OC₁₋₆-alkylhalo.

3. A compound according claim 2, wherein R⁴ is selected from H, alkyl, cycloalkyl, or cycloalkenyl.

4. A compound according to claim 3, wherein R⁴ is selected from H or alkyl.

5. A compound according to claim 1, wherein R⁹ to R¹² are independently selected from H, alkyl, alkenyl, alkynyl, cycloalkyl, cycloalkenyl, aryl, heteroaryl, alkylaryl, or alkylheteroaryl.

6. A compound according to claim 5, wherein R⁹ to R¹² are independently selected from H, alkyl or cycloalkyl.

7. A compound according to claim 1, wherein the compound is a pharmaceutically-acceptable salt, optical isomer, or combination thereof.

8. A compound according to claim 1, wherein the pharmaceutically-acceptable salt comprises an acid addition salt or a basic addition salt.

9. A compound according to claim 8, wherein the acid addition salt is formed from hydrochloric acid, hydrobromic acid, sulfuric acid, phosphoric acid, acid metal salt, monocarboxylic acids, dicarboxylic acids, or tricarboxylic acids.

10. A compound according to claim 1, wherein the compound of Formula I comprises a compound of Formula IA: 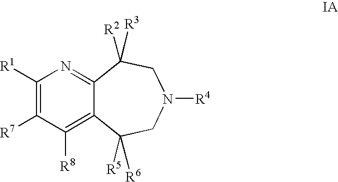
wherein:
R² and R⁵ are independently selected from H, alkyl, alkylene-O-alkyl, C(O)Oalkyl, C(O)N(H)alkyl, haloalkyl, halogen or CH₂OH; and
R³ and R⁶ are each H; or R² and R³ and/or R⁵ and R⁶, together with the carbon atom to which they are attached form a cycloalkyl group;
and/or a pharmaceutically-acceptable salt, hydrate, solvate, isoform, tautomer, optical isomer, or combination thereof.

11. A compound according to claim 1, wherein the compound of Formula I comprises a compound of Formula IB: 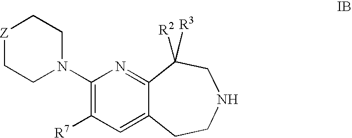
wherein
Z is selected from CR¹⁴R¹⁵, O, NR¹⁶, C═O, S═O, SO₂ or S; and
R¹⁴ to R¹⁶ are independently selected from H, halo, hydroxy, cyano, nitro, alkyl, alkoxy, CH₂OH, haloalkyl, O-haloalkyl, hydroxyalkyl, cyanoalkyl, alkenyl, alkynyl, cycloalkyl, cycloalkenyl, heterocycloalkyl, heterocycloalkenyl, aryl, heteroaryl, alkylaryl, alkylheteroaryl, O-cycloalkyl, O-heterocycloalkyl, alkylene-O-alkyl, alkylene-O-cycloalkyl, alkylene-O-heterocycloalkyl, alkylene-O-alkylene-cycloalkyl, alkylene-O-alkylene-heterocycloalkyl, S-alkyl, S(O)-alkyl, S(O)₂-alkyl, S-cycloalkyl, S(O)-cycloalkyl, S(O)₂-cycloalkyl, S-heterocycloalkyl, S(O)-heterocycloalkyl, S(O)₂-heterocycloalkyl, O-aryl, O-heteroaryl, N(H)alkyl, N(alkyl)alkyl, N(H)-aryl, N(alkyl)-aryl, N(H)-heteroaryl, N(alkyl)-heteroaryl, alkylene-O-aryl, alkylene-O-heteroaryl, alkylene-O-alkylene-aryl, alkylene-O-alkylene-heteroaryl, S-aryl, S-heteroaryl, S(O)-aryl, S(O)-heteroaryl, S(O)₂-aryl, S(O)₂-heteroaryl, C(O)alkyl, OC(O)alkyl, C(O)Oalkyl, C(O)N(H)alkyl, C(O)N(alkyl)alkyl, S(O)₂N(H)alkyl or S(O)₂N(alkyl)alkyl;
and/or a pharmaceutically-acceptable salt, hydrate, solvate, isoform, tautomer, optical isomer, or combination thereof.

12. A compound according to claim 11, wherein R⁷ is selected from H or halo; R² and R³ are independently selected from H or alkyl; R¹⁴ and R¹⁵ are independently selected from H, halo, or alkyl; and R¹⁶ is selected from H or alkyl.

13. A compound according to claim 12, wherein the halo is bromo, chloro, or fluoro.

14. A compound according to claim 13, wherein Z is CR¹⁴R¹⁵ wherein R¹⁴ is H or fluoro and R¹⁵ is fluoro.

15. A compound according to claim 14, wherein R⁷ is H and Z is CR¹⁴R¹⁵, wherein R¹⁴ is H and R¹⁵ is fluoro.

16. A compound according to claim 1, wherein the compound of Formula I comprises a compound of Formula IC: 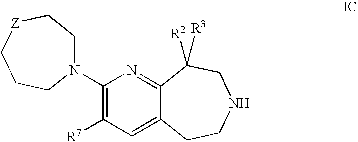
wherein:
Z is selected from CR¹⁴R¹⁵, O, NR⁶, C═O, S═O, SO₂ or S; and
R¹⁴ to R¹⁶ are independently selected from H, halo, hydroxy, cyano, nitro, alkyl, alkoxy, CH₂OH, haloalkyl, O-haloalkyl, hydroxyalkyl, cyanoalkyl, alkenyl, alkynyl, cycloalkyl, cycloalkenyl, heterocycloalkyl, heterocycloalkenyl, aryl, heteroaryl, alkylaryl, alkylheteroaryl, O-cycloalkyl, O-heterocycloalkyl, alkylene-O-alkyl, alkylene-O-cycloalkyl, alkylene-O-heterocycloalkyl, alkylene-O-alkylene-cycloalkyl, alkylene-O-alkylene-heterocycloalkyl, S-alkyl, S(O)-alkyl, S(O)₂-alkyl, S-cycloalkyl, S(O)-cycloalkyl, S(O)₂-cycloalkyl, S-heterocycloalkyl, S(O)-heterocycloalkyl, S(O)₂-heterocycloalkyl, O-aryl, O-heteroaryl, N(H)alkyl, N(alkyl)alkyl, N(H)-aryl, N(alkyl)-aryl, N(H)-heteroaryl, N(alkyl)-heteroaryl, alkylene-O-aryl, alkylene-O-heteroaryl, alkylene-O-alkylene-aryl, alkylene-O-alkylene-heteroaryl, S-aryl, S-heteroaryl, S(O)-aryl, S(O)-heteroaryl, S(O)₂-aryl, S(O)₂-heteroaryl, C(O)alkyl, OC(O)alkyl, C(O)Oalkyl, C(O)N(H)alkyl, C(O)N(alkyl)alkyl, S(O)₂N(H)alkyl or S(O)₂N(alkyl)alkyl;
and/or a pharmaceutically-acceptable salt, hydrate, solvate, isoform, tautomer, optical isomer, or combination thereof.

17. A compound according to claim 16, wherein R⁷ is selected from H or halo; R² and R³ are independently selected from H or alkyl; R¹⁴ and R¹⁵ are independently selected from H, halo, or alkyl; and R¹⁶ is selected from H or alkyl.

18. A compound according to claim 17, wherein the halo is bromo, chloro, or fluoro.

19. A compound according to claim 18, wherein Z is O.

20. A compound according to claim 1, wherein the compound of Formula I comprises a compound of Formula II:

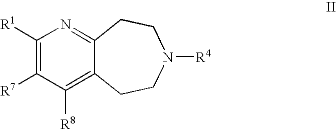

21. A compound according to claim 1, wherein the compound of Formula I comprises a compound of Formula III:

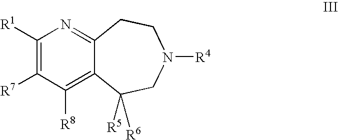

22. A compound according to claim 1, wherein the compound of Formula I comprises a compound of Formula IV:

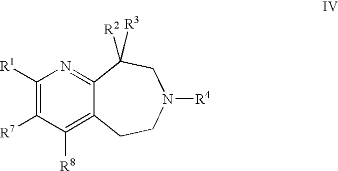

23. A compound according to claim 22, wherein R⁷ is selected from H, alkyl or halo; R², R³ and R⁸ are independently selected from H or alkyl; R⁴ is selected from H or alkyl; and R¹ is selected from H, alkyl, alkoxy, CH₂OH, alkenyl, cycloalkyl, heterocycloalkyl, aryl, or heteroaryl.

24. A compound selected from:
(9R)-2-(4,4-difluoropiperidin-1-yl)-9-methyl-6,7,8,9-tetrahydro-5H-pyrido[2,3d]azepine;
(9R)-2-(4-fluoropiperidin-1-yl)-9-methyl-6,7,8,9-tetrahydro-5H-pyrido[2,3-d]azepine;
(9R)-9-methyl-2-(1,4-oxazepan-4-yl)-6,7,8,9-tetrahydro-5H-pyrido[2,3-d]azepine;
(9R)-9-methyl-2-morpholin-4-yl-6,7,8,9-tetrahydro-5H-pyrido[2,3-d]azepine;
(9R)-9-methyl-2-piperidin-1-yl-6,7,8,9-tetrahydro-5H-pyrido[2,3-d]azepine;
(9R)-N-ethyl-N,9-dimethyl-6,7,8,9-tetrahydro-5H-pyrido[2,3-d]azepin-2-amine;
(9S)-2-(4,4-difluoropiperidin-1-yl)-9-methyl-6,7,8,9-tetrahydro-5H-pyrido[2,3-d]azepine;
(9S)-2-(4-fluoropiperidin-1-yl)-9-methyl-6,7,8,9-tetrahydro-5H-pyrido[2,3-d]azepine;
(9S)-9-methyl-2-(1,4-oxazepan-4-yl)-6,7,8,9-tetrahydro-5H-pyrido[2,3-d]azepine
(9S)-9-methyl-2-morpholin-4-yl-6,7,8,9-tetrahydro-5H-pyrido[2,3-d]azepine;
(9S)-9-methyl-2-piperidin-1-yl-6,7,8,9-tetrahydro-5H-pyrido[2,3-d]azepine;
(9S)-N-ethyl-N,9-dimethyl-6,7,8,9-tetrahydro-5H-pyrido[2,3-d]azepin-2-amine;
2-(1,4-diazepan-1-yl)-6,7,8,9-tetrahydro-5H-pyrido[2,3-d]azepine;
2-(1,4-oxazepan-4-yl)-6,7,8,9-tetrahydro-5H-pyrido[2,3-d]azepine;
2-(1-oxidothiomorpholin-4-yl)-6,7,8,9-tetrahydro-5H-pyrido[2,3-d]azepine;
2-(3-thienyl)-6,7,8,9-tetrahydro-5H-pyrido[2,3-d]azepine;
2-(4,4-difluoroazepan-1-yl)-6,7,8,9-tetrahydro-5H-pyrido[2,3-d]azepine;
2-(4,4-difluoroazepan-1-yl)-9-methyl-6,7,8,9-tetrahydro-5H-pyrido[2,3-d]azepine;
2-(4,4-difluoropiperidin-1-yl)-6,7,8,9-tetrahydro-5H-pyrido[2,3-d]azepine;
2-(4,4-difluoropiperidin-1-yl)-9-methyl-6,7,8,9-tetrahydro-5H-pyrido[2,3-d]azepine;
2-(4-fluoropiperidin-1-yl)-6,7,8,9-tetrahydro-5H-pyrido[2,3-d]azepine;
2-(4-fluoropiperidin-1-yl)-9-methyl-6,7,8,9-tetrahydro-5H-pyrido[2,3-d]azepine;
2-(4-methylpiperazin-1-yl)-6,7,8,9-tetrahydro-5H-pyrido[2,3-d]azepine;
2-(8-azabicyclo[3.2.1]oct-8-yl)-6,7,8,9-tetrahydro-5H-pyrido[2,3-d]azepine;
2-(trifluoromethyl)-6,7,8,9-tetrahydro-5H-pyrido[2,3-d]azepine;
2,9-dimethyl-6,7,8,9-tetrahydro-5H-pyrido[2,3-d]azepine;
2-[methyl(6,7,8,9-tetrahydro-5H-pyrido[2,3-d]azepin-2-yl)amino]ethanol;
2-azepan-1-yl-6,7,8,9-tetrahydro-5H-pyrido[2,3-d]azepine;
2-azepan-1-yl-9-isopropyl-6,7,8,9-tetrahydro-5H-pyrido[2,3-d]azepine;
2-azepan-1-yl-9-methyl-6,7,8,9-tetrahydro-5H-pyrido[2,3-d]azepine;
2-cyclopropyl-6,7,8,9-tetrahydro-5H-pyrido[2,3-d]azepine;
2-cyclopropyl-9-methyl-6,7,8,9-tetrahydro-5H-pyrido[2,3-d]azepine;
2-isopropenyl-6,7,8,9-tetrahydro-5H-pyrido[2,3-d]azepine;
2-isopropenyl-9-methyl-6,7,8,9-tetrahydro-5H-pyrido[2,3-d]azepine;
2-isopropyl-6,7,8,9-tetrahydro-5H-pyrido[2,3-d]azepine;
2-isopropyl-9-methyl-6,7,8,9-tetrahydro-5H-pyrido[2,3-d]azepine;
2-methoxy-6,7,8,9-tetrahydro-5H-pyrido[2,3-d]azepine;
2-methoxy-9-methyl-6,7,8,9-tetrahydro-5H-pyrido[2,3-d]azepine;
2-methyl-6,7,8,9-tetrahydro-5H-pyrido[2,3-d]azepine;
2-morpholin-4-yl-6,7,8,9-tetrahydro-5H-pyrido[2,3-d]azepine;
2-piperazin-1-yl-6,7,8,9-tetrahydro-5H-pyrido[2,3-d]azepine;
2-piperidin-1-yl-6,7,8,9-tetrahydro-5H-pyrido[2,3-d]azepine;
2-pyrrolidin-1-yl-6,7,8,9-tetrahydro-5H-pyrido[2,3-d]azepine;
2-tert-butyl-6,7,8,9-tetrahydro-5H-pyrido[2,3-d]azepine;
2-thiomorpholin-4-yl-6,7,8,9-tetrahydro-5H-pyrido[2,3-d]azepine;
2-vinyl-6,7,8,9-tetrahydro-5H-pyrido[2,3-d]azepine;
3-bromo-2-methoxy-6,7,8,9-tetrahydro-5H-pyrido[2,3-d]azepine;
3-bromo-2-morpholin-4-yl-6,7,8,9-tetrahydro-5H-pyrido[2,3-d]azepine;
3-bromo-2-piperidin-1-yl-6,7,8,9-tetrahydro-5H-pyrido[2,3-d]azepine;
3-bromo-N-ethyl-N-methyl-6,7,8,9-tetrahydro-5H-pyrido[2,3-d]azepin-2-amine;
3-chloro-2-(1,4-oxazepan-4-yl)-6,7,8,9-tetrahydro-5H-pyrido[2,3-d]azepine;
3-chloro-2-(4,4-difluoropiperidin-1-yl)-6,7,8,9-tetrahydro-5H-pyrido[2,3-d]azepine;
3-chloro-2-(4-fluoropiperidin-1-yl)-6,7,8,9-tetrahydro-5H-pyrido[2,3-d]azepine;
3-chloro-2-morpholin-4-yl-6,7,8,9-tetrahydro-5H-pyrido[2,3-d]azepine;
3-chloro-2-piperidin-1-yl-6,7,8,9-tetrahydro-5H-pyrido[2,3-d]azepine;
6,7,8,9-tetrahydro-5H-pyrido[2,3-d]azepine-2-carbaldehyde;
6,7,8,9-tetrahydro-5H-pyrido[2,3-d]azepine-2-carbonitrile;
9-ethyl-2-morpholin-4-yl-6,7,8,9-tetrahydro-5H-pyrido[2,3-d]azepine;
9-ethyl-2-piperidin-1-yl-6,7,8,9-tetrahydro-5H-pyrido[2,3-d]azepine;
9-isopropyl-2-morpholin-4-yl-6,7,8,9-tetrahydro-5H-pyrido[2,3-d]azepine;
9-isopropyl-2-piperidin-1-yl-6,7,8,9-tetrahydro-5H-pyrido[2,3-d]azepine;
9-methyl-2-(1,4-oxazepan-4-yl)-6,7,8,9-tetrahydro-5H-pyrido[2,3-d]azepine;
9-methyl-2-morpholin-4-yl-6,7,8,9-tetrahydro-5H-pyrido[2,3-d]azepine;
9-methyl-2-piperidin-1-yl-6,7,8,9-tetrahydro-5H-pyrido[2,3-d]azepine;
ethyl 4-(6,7,8,9-tetrahydro-5H-pyrido[2,3-d]azepin-2-yl)piperazine-1-carboxylate;
N,9-diethyl-N-methyl-6,7,8,9-tetrahydro-5H-pyrido[2,3-d]azepin-2-amine;
N,N,9-trimethyl-6,7,8,9-tetrahydro-5H-pyrido[2,3-d]azepin-2-amine;
N,N-diallyl-6,7,8,9-tetrahydro-5H-pyrido[2,3-d]azepin-2-amine;
N,N-diethyl-6,7,8,9-tetrahydro-5H-pyrido[2,3-d]azepin-2-amine;
N,N-dimethyl-6,7,8,9-tetrahydro-5H-pyrido[2,3-d]azepin-2-amine;
N,N-dimethyl-6,7,8,9-tetrahydro-5H-pyrido[2,3-d]azepine-2-carboxamide;
N-benzyl-N-methyl-6,7,8,9-tetrahydro-5H-pyrido[2,3-d]azepin-2-amine;
N-ethyl-N,9-dimethyl-6,7,8,9-tetrahydro-5H-pyrido[2,3-d]azepin-2-amine;
N-ethyl-N-methyl-6,7,8,9-tetrahydro-5H-pyrido[2,3-d]azepin-2-amine;
N-isopropyl-N-methyl-6,7,8,9-tetrahydro-5H-pyrido[2,3-d]azepin-2-amine;
N-methyl-6,7,8,9-tetrahydro-5H-pyrido[2,3-d]azepin-2-amine;
N-methyl-N-(6,7,8,9-tetrahydro-5H-pyrido[2,3-d]azepin-2-yl)acetamide;
2-Phenyl-6,7,8,9-tetrahydro-5H-pyrido[2,3-d]azepine;
6,7,8,9-Tetrahydro-5H-pyrido[2,3-d]azepine-2-carbonitrile;
2-Chloro-6,7,8,9-tetrahydro-5H-pyrido[2,3-d]azepine;
[2-(6-Methoxy-3-methyl-pyridin-2-yl)-ethyl]-methyl-amine; and/or
7,8,9-Tetrahydro-5H-pyrido[2,3-d]azepin-2-ol;
and/or a pharmaceutically-acceptable salt, hydrate, solvate, isoform, tautomer, optical isomer, or combination thereof.

25. A compound selected from:
(9R)-2-(4,4-difluoropiperidin-1-yl)-9-methyl-6,7,8,9-tetrahydro-5H-pyrido[2,3d]azepine;
(9R)-2-(4-fluoropiperidin-1-yl)-9-methyl-6,7,8,9-tetrahydro-5H-pyrido[2,3-d]azepine;
(9R)-9-methyl-2-(1,4-oxazepan-4-yl)-6,7,8,9-tetrahydro-5H-pyrido[2,3-d]azepine;
(9R)-9-methyl-2-morpholin-4-yl-6,7,8,9-tetrahydro-5H-pyrido[2,3-d]azepine;
(9R)-9-methyl-2-piperidin-1-yl-6,7,8,9-tetrahydro-5H-pyrido[2,3-d]azepine;
(9R)-N-ethyl-N,9-dimethyl-6,7,8,9-tetrahydro-5H-pyrido[2,3-d]azepin-2-amine;
(9S)-2-(4,4-difluoropiperidin-1-yl)-9-methyl-6,7,8,9-tetrahydro-5H-pyrido[2,3-d]azepine;
(9S)-2-(4-fluoropiperidin-1-yl)-9-methyl-6,7,8,9-tetrahydro-5H-pyrido[2,3-d]azepine;
(9S)-9-methyl-2-(1,4-oxazepan-4-yl)-6,7,8,9-tetrahydro-5H-pyrido[2,3-d]azepine
(9S)-9-methyl-2-morpholin-4-yl-6,7,8,9-tetrahydro-5H-pyrido[2,3-d]azepine;
(9S)-9-methyl-2-piperidin-1-yl-6,7,8,9-tetrahydro-5H-pyrido[2,3-d]azepine;
(9S)-N-ethyl-N,9-dimethyl-6,7,8,9-tetrahydro-5H-pyrido[2,3-d]azepin-2-amine;
2-(1,4-diazepan-1-yl)-6,7,8,9-tetrahydro-5H-pyrido[2,3-d]azepine;
2-(1,4-oxazepan-4-yl)-6,7,8,9-tetrahydro-5H-pyrido[2,3-d]azepine;
2-(4,4-difluoroazepan-1-yl)-6,7,8,9-tetrahydro-5H-pyrido[2,3-d]azepine;
2-(4,4-difluoropiperidin-1-yl)-6,7,8,9-tetrahydro-5H-pyrido[2,3-d]azepine;
2-(4-fluoropiperidin-1-yl)-6,7,8,9-tetrahydro-5H-pyrido[2,3-d]azepine;
2-azepan-1-yl-6,7,8,9-tetrahydro-5H-pyrido[2,3-d]azepine;
2-azepan-1-yl-9-methyl-6,7,8,9-tetrahydro-5H-pyrido[2,3-d]azepine;
2-cyclopropyl-9-methyl-6,7,8,9-tetrahydro-5H-pyrido[2,3-d]azepine;
2-isopropenyl-6,7,8,9-tetrahydro-5H-pyrido[2,3-d]azepine;
2-isopropenyl-9-methyl-6,7,8,9-tetrahydro-5H-pyrido[2,3-d]azepine;
2-isopropyl-6,7,8,9-tetrahydro-5H-pyrido[2,3-d]azepine;
2-isopropyl-9-methyl-6,7,8,9-tetrahydro-5H-pyrido[2,3-d]azepine;
2-morpholin-4-yl-6,7,8,9-tetrahydro-5H-pyrido[2,3-d]azepine;
2-piperidin-1-yl-6,7,8,9-tetrahydro-5H-pyrido[2,3-d]azepine;
2-tert-butyl-6,7,8,9-tetrahydro-5H-pyrido[2,3-d]azepine;
2-thiomorpholin-4-yl-6,7,8,9-tetrahydro-5H-pyrido[2,3-d]azepine;
3-bromo-2-methoxy-6,7,8,9-tetrahydro-5H-pyrido[2,3-d]azepine;
3-chloro-2-(1,4-oxazepan-4-yl)-6,7,8,9-tetrahydro-5H-pyrido[2,3-d]azepine;
3-chloro-2-(4,4-difluoropiperidin-1-yl)-6,7,8,9-tetrahydro-5H-pyrido[2,3-d]azepine;
3-chloro-2-(4-fluoropiperidin-1-yl)-6,7,8,9-tetrahydro-5H-pyrido[2,3-d]azepine;
3-chloro-2-morpholin-4-yl-6,7,8,9-tetrahydro-5H-pyrido[2,3-d]azepine;
3-chloro-2-piperidin-1-yl-6,7,8,9-tetrahydro-5H-pyrido[2,3-d]azepine;
9-isopropyl-2-piperidin-1-yl-6,7,8,9-tetrahydro-5H-pyrido[2,3-d]azepine;
9-methyl-2-morpholin-4-yl-6,7,8,9-tetrahydro-5H-pyrido[2,3-d]azepine;
9-methyl-2-piperidin-1-yl-6,7,8,9-tetrahydro-5H-pyrido[2,3-d]azepine;
N,N-diethyl-6,7,8,9-tetrahydro-5H-pyrido[2,3-d]azepin-2-amine;
N,N-dimethyl-6,7,8,9-tetrahydro-5H-pyrido[2,3-d]azepin-2-amine;
N-ethyl-N,9-dimethyl-6,7,8,9-tetrahydro-5H-pyrido[2,3-d]azepin-2-amine; and/or
N-ethyl-N-methyl-6,7,8,9-tetrahydro-5H-pyrido[2,3-d]azepin-2-amine;
and/or a pharmaceutically-acceptable salt, hydrate, solvate, isoform, tautomer, optical isomer, or combination thereof.

26. A compound according to claim 1, wherein the compound has an EC₅₀ for a human 5-HT_{2C} receptor selected from less than 1000 nM, less than 500 nM, less than 300 nM, or less than 100 nM.

27. A pharmaceutical composition comprising a compound according to claim 1 and at least one pharmaceutically acceptable carrier and/or excipient.

28-37. (canceled)

38. A method for treating a 5-HT_{2C} receptor-mediated disorder in a mammal, comprising administering to the mammal a therapeutically effective amount of a compound according to claim 1.

39. The method according to claim 38, wherein the mammal is a human.

40. The method according to claim 38, wherein the disorder is selected from the group consisting of a depressive disorder, an anxiety disorder, including panic attack, agoraphobia, and specific or social phobia, bipolar disorder, post-traumatic stress, an eating disorder, obesity, a gastrointestinal disorder, alcoholism, drug addiction, schizophrenia, a psychotic disorder, a sleep disorder, including sleep apnea, migraine, sexual dysfunction, a central nervous system disorder, including trauma, stroke and spinal cord injury, a cardiovascular disorder, diabetes insipidus, obsessive compulsive disorder, premenstrual tension, chronic fatigue syndrome, age-related memory disorder, personality disorder and raised intracranial pressure.

41. The method according to claim 40, wherein the disorder is selected from the group consisting of obesity, schizophrenia, epilepsy, a depressive disorder, panic attack, alcoholism, drug addiction or obsessive compulsive disorder.

42. The method according to claim 38, wherein the compound is administered orally and/or parenterally.

43. The method according to claim 42, wherein the compound is administered intravenously and/or intraperitoneally.

44-50. (canceled)

51. A method for decreasing food intake in a mammal comprising administering to the mammal a therapeutically effective amount of a compound according to claim 1.

52. A method of controlling weight gain in a mammal comprising administering to the mammal a therapeutically effective amount of a compound according to claim 1.

53. Use of a compound according to claim 1 for the manufacture of a medicament for decreasing food intake or controlling weight gain in a mammal.

54. Use of a compound according to claim 1 for decreasing food intake or controlling weight gain in a mammal.
